(19) United States
(12) Patent Application Publication
(10) Pub. No.: US 2023/0332230 A1
(43) Pub. Date: Oct. 19, 2023

(54) BIOMARKERS AND USES THEREOF IN THE TREATMENT OF CHRONIC HEPATITIS B INFECTION

(71) Applicant: Janssen Sciences Ireland Unlimited Company, Co Cork (IE)

(72) Inventors: Marianne Isabelle TUEFFERD, Berchem (BE); Marjolein CRABBE, Kortrijk (BE); Jacques Armand Henri BOLLEKENS, lxelles (BE); Jeroen Marcel Maria Roger AERSSENS, Nieuwrode (Holsbeek) (BE); Alessandro DI CARA, Beaumont (FR)

(21) Appl. No.: 17/757,317

(22) PCT Filed: Feb. 5, 2021
(86) PCT No.: PCT/IB2021/050970

§ 371 Date: Jun. 14, 2022

Related U.S. Application Data
(60) Provisional application No. 63/056,847, filed on Jul. 27, 2020.
(60) Provisional application No. 63/038,188, filed on Jun. 12, 2020.
(60) Provisional application No. 62/970,903, filed on Feb. 6, 2020.

(51) Int. Cl. C12Q 1/6883; C12Q 1/70

(52) U.S. Cl. CPC C12Q 1/6883; C12Q 1/706; A61K 31/675; C12Q 2600/106; C12Q 2600/156

(57) ABSTRACT

Single nucleotide polymorphisms (SNPs) that are indicative of relapse after a HBV direct-acting antiviral agent (DAA) treatment, such as a NUC treatment in a chronic hepatitis B (CHB) infected subject are described. Also described are methods of using the SNPs in predicting the relapse in the HBV DAA treatment of CHB infection.

### CROSS REFERENCE TO RELATED APPLICATION

This application claims priority to U.S. Provisional Pat. Application No. 62/970,903, filed on Feb. 6, 2020, U.S. Provisional Pat. Application No. 63/038,188, filed Jun. 12, 2020, and U.S. Provisional Pat. Application No. 63/056,847, filed Jul. 27, 2020, the disclosures of which are incorporated herein by reference.

### FIELD OF THE INVENTION

The present invention is directed generally to biomarkers and related uses in the treatment of chronic hepatitis B infection.

### BACKGROUND OF THE INVENTION

Chronic hepatitis B virus (HBV) infection affects about 400 million people worldwide and is among the world’s leading causes of death. Association for the Study of Liver Disease (AASLD) guidelines recommend treatment for patients who present with levels of serum HBV DNA over 2,000 IU/mL and/or with elevated alanine aminotransferase (ALT) levels (>2 times upper limit of normal).

Antiviral therapy of chronic hepatitis B (CHB) is aimed to decrease the liver-related morbidity and mortality. The achievement of a sustained suppression of HBV replication has been associated with normalization of serum alanine transaminase (ALT), loss of hepatitis B e-antigen (HBeAg) with or without detection of anti-HBe, and improvement in liver histology. This goal can be achieved by, for example, short-term treatment with pegylated interferon (Peg-IFN) or long-term suppressive therapy with oral nucleotide or nucleoside analogues (NUCs) (Lok & McMahon, Hepatology, 2009, 50: 661-662; EASL clinical practice guidelines: management of chronic hepatitis B, J. Hepatol., 2012, 57:167-185). Recently, oral administration of NUCs has become the most popular treatment strategy worldwide given the excellent efficacy and safety of third-generation NUC such as entecavir and tenofovir, not only in registration trials but also in clinical practice.

Oral anti-viral NUCs can be prescribed as once-daily oral dosing with minimal side effects, and are very effective in viral suppression and normalization of liver enzymes. However, most patients require long-term therapy and virological relapse is common after premature cessation of therapy (Ahn, et al., Hepatol. Int., 2010, 4: 386-95; van Nunen, et al., Gut., 2003, 52: 420-442).

Clearance of hepatitis B surface antigen (HBsAg) is the ideal endpoint to stop treatment, but its occurrence is usually lower than 5% in 5 years with anti-viral therapy. Recommendations about stopping treatment depend on different groups of CHB patients. Nonetheless, approximately 25% to 50% of the patients may still develop hepatitis relapse after stopping NUC therapy even if these recommendations are followed (Fung, et al., Am. J. Gastroenterol 2009; 104: 1940-6; Hadziyannis, et al., Hepatology 2006, 1: 231A).

Therefore, it would be desirable to be able to predict a patient’s response to CHB therapy, and then accordingly adjust a treatment strategy right from the beginning, in order to minimize the risk of relapse after termination of treatment. In particular, it is preferred that the prediction is based on the detection of biomarkers related to CHB treatment.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

The present invention provides identification and uses of biomarkers, especially single nucleotide polymorphisms (SNPs), in the treatment of CHB patients.

In one general aspect, the application relates to an isolated set of probes for use in treating a chronic hepatitis B (CHB) infection in a subject in need thereof, wherein the set of probes detects a panel of single nucleotide polymorphisms (SNPs), and the panel comprises one or more SNPs associated with time to relapse, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof.

In certain embodiments, the one or more SNPs are associated with time to relapse with a p-value of 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542, or a complementary sequence thereof.

In certain embodiments, the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof.

In certain embodiments, the one or more SNPs are selected from the group consisting of rs4668818, rs948006, rs2934456, rs75876539, rs8050261, and rs1542951, or a complementary sequence thereof.

In another general aspect, the application relates to an isolated set of probes capable of detecting a panel of SNPs, and the panel comprises one or more SNPs associated with time to relapse, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof.

In certain embodiments, the one or more SNPs are associated with time to relapse with a p-value of 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542, or a complementary sequence thereof.

In certain embodiments, the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof.

In certain embodiments, the one or more SNPs are selected from the group consisting of rs4668818, rs948006, rs2934456, rs75876539, rs8050261, and rs1542951, or a complementary sequence thereof.

In one general aspect, the application relates to a method of treating a chronic hepatitis B (CHB) infection in a subject in need thereof, the method comprising:
a. administering to the subject a therapeutically effective amount of a HBV direct-acting antiviral agent (DAA) to treat the CHB infection;
b. discontinuing the HBV DAA treatment when the CHB infection is suppressed in the subject;
c. detecting in a biological sample obtained from the subject the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof; and
d. monitoring relapse in the subject two years or later after the discontinuation of the HBV DAA treatment, if the panel of the one or more SNPs is detected in the biological sample; or monitoring relapse in the subject in the first two years after the discontinuation of the HBV DAA treatment, if none of the one or more SNPs is detected in the biological sample.

In another general aspect, the application relates to a method of treating a chronic hepatitis B (CHB) infection in a subject in need thereof, the method comprising:
a. detecting in a biological sample obtained from the subject the presence of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof; and
b. administering to the subject a therapeutically effective amount of a HBV direct-acting antiviral agent (DAA) if the panel of the one or more SNPs is detected in the biological sample, or administering to the subject a therapeutically effective amount of a non-DAA agent if none of the SNPs is detected in the biological sample.

In certain embodiments, the HBV DAA is a nucleotide or nucleoside (NUC) selected from the group consisting of tenofovir, entecavir, lamivudine, adefovir, and telbivudine.

In certain embodiments, the subject discontinues the HBV DAA treatment when the subject achieves HBV DNA < 60 IU/mL, ALT < 80 U/L, or HBeAg negative.

In certain embodiments, the subject has no virological relapse or clinical relapse at or after 3 months, 6 months, 12 months, 18 months, 24 months, or 36 months after the discontinuation of the HBV DAA treatment, or anytime in between, and wherein the virological relapse is identified as HBV DNA ≥ 2000 IU/ml or HBeAg positive, and the clinical relapse is identified as i) HBV DNA ≥ 2000 IU/ml or HBeAg positive, and ii) ALT ≥ 80 U/L.

In another general aspect, the application relates to a method of treating a chronic hepatitis B (CHB) infection in a subject in need thereof, the method comprising:
a. administering to the subject a therapeutically effective amount of a HBV direct-acting antiviral agent (DAA) to treat the CHB;
b. detecting in a biological sample obtained from the subject the presence of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof; and
c. continuing treating the subject with the HBV DAA if the panel of the one or more SNPs is detected in the biological sample, or switching from the HBV DAA treatment to a non-DAA treatment if none of the SNPs is detected in the biological sample.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof.

In certain embodiments, the one or more single nucleotide polymorphisms (SNPs) is selected from the group consisting of rs4668818, rs948006, rs2934456, rs77586835, rs75876539, rs8050261, rs1542951, rs231770, and rs9277535, or a complementary sequence thereof.

In certain embodiments, the HBV DAA is a nucleotide or nucleoside (NUC) selected from the group consisting of tenofovir, entecavir, lamivudine, adefovir, and telbivudine.

In certain embodiments, the subject discontinues the HBV DAA treatment when the subject achieves HBV DNA < 60 IU/mL, ALT < 80 U/L, or HBeAg negative.

In certain embodiments, the subject has no virological relapse or clinical relapse at or after 3 months, 6 months, 12 months, 18 months, 24 months, or 36 months after the discontinuation of the HBV DAA treatment, or anytime in between, and wherein the virological relapse is identified as HBV DNA ≥ 2000 IU/ml or HBeAg positive, and the clinical relapse is identified as i) HBV DNA ≥ 2000 IU/ml or HBeAg positive, and ii) ALT ≥ 80 U/L.

In certain embodiments, the sample is selected from a tissue sample, a cellular sample, a blood sample. Preferably, the sample is a blood sample.

In another general aspect, the application relates to a panel of SNPs for predicting a relapse after discontinuation of a HBV DAA treatment of a chronic hepatitis B (CHB) infection in a subject in need thereof, wherein the panel comprises one or more SNPs described in the application in a biological sample of the subject.

In another aspect, provided herein is a microarray for the assessment of the panel of isolated biomarkers disclosed herein, which comprises a combination of molecules on a substrate, wherein said molecules are used for assaying the SNPs. In some embodiments, the molecules can be oligonucleotides or polypeptides.

In yet another aspect, provided herein is a companion diagnostic test for a treatment of CHB using a panel of isolated biomarkers comprising one, two, three, four or more SNPs described herein. In some embodiments, the companion diagnostic test can comprise: a) obtaining a biological sample from a subject that is undergoing a CHB treatment or is considered for a CHB treatment; b) isolating genomic DNA from said biological sample; c) assaying a panel of biomarkers according to embodiment of the application; d) generating an output with a computer algorithm based on the assay results of said panel of biomarkers; and/or e) determining the likely for relapse of said subject to the CHB treatment. In some embodiments, the SNPs can be assayed by sequencing, capillary electrophoresis, mass spectrometry, single-strand conformation polymorphism (SSCP), electrochemical analysis, denaturing HPLC and gel electrophoresis, restriction fragment length polymorphism, hybridization analysis, single-base extension, and/or microarray.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 is the study diagram showing treatment and follow-up after termination of treatment.

FIGS. 2A-D demonstrate HBV DNA shaped by corresponding ALT per clinical relapse and end of study event group:
FIG. 2A: patients without clinical relapse who completed the study;
FIG. 2B: patients with clinical relapse who completed the study;
FIG. 2C: patients without clinical relapse who restarted nuclos(t)ide analogue (NA) therapy; and
FIG. 2D: patients with clinical relapse who restarted NT therapy.

FIGS. 3A-H show the levels of HBV DNA and ALT for the 8 transient clinical relapsers:
FIG. 3A: the levels of HBV DNA and ALT in the 1^{st} transient clinical relapse;
FIG. 3B: the levels of HBV DNA and ALT in the 2^{nd} transient clinical relapse;
FIG. 3C: the levels of HBV DNA and ALT in the 3^{rd} transient clinical relapse;
FIG. 3D: the levels of HBV DNA and ALT in the 4^{th} transient clinical relapse;
FIG. 3E: the levels of HBV DNA and ALT in the 5^{th} transient clinical relapse;
FIG. 3F: the levels of HBV DNA and ALT in the 6^{th} transient clinical relapse;
FIG. 3G: the levels of HBV DNA and ALT in the 7^{th} transient clinical relapse; and
FIG. 3H: the levels of HBV DNA and ALT in the 8^{th} transient clinical relapse.

FIGS. 4A-D demonstrate HBV DNA shaped by corresponding ALT per clinical relapse and treatment regimen group:
FIG. 4A: patients without clinical relapse treated with entecavir;
FIG. 4B: patients with clinical relapse treated with entecavir;
FIG. 4C: patients without clinical relapse treated with tenofovir; and
FIG. 4D: patients with clinical relapse treated with tenofovir.

FIGS. 5A-B demonstrate HBsAg level per clinical relapse:
FIG. 5A: patients without clinical relapse; and
FIG. 5B: patients with clinical relapse.

FIGS. 6A-C demonstrate cumulative incidence of clinical relapse associated to various factors:
FIG. 6A demonstrates cumulative incidence of clinical relapse associated to gender;
FIG. 6B demonstrates cumulative incidence of clinical relapse associated to treatment regimen; and
FIG. 6C demonstrates cumulative incidence of clinical relapse associated to end-of-treatment HBsAg.

FIG. 7 shows the distribution of age per prior HBeAg status shaped by gender.

FIGS. 8A-C demonstrate cumulative incidence of virological relapse associated to various factors:
FIG. 8A demonstrates cumulative incidence of virological relapse associated to prior HBeAg status;
FIG. 8B demonstrates cumulative incidence of virological relapse associated to treatment regimen, and
FIG. 8C demonstrates cumulative incidence of virological relapse associated to end-of-treatment HBsAg.

FIGS. 9A-C show sustained clinical response:
FIG. 9A shows HBeAg status prior to or at the start of the treatment;
FIG. 9B shows HBsAg status at the end-of-treatment; and
FIG. 9C shows HBV DNA level one month after the treatment.

FIG. 10 shows that gene RBFOX1is associated with time to clinical relapse.

FIG. 11 shows that genotype rs8050261 G/G (RBFOX1) is protective for clinical relapse.

FIG. 12 shows that gene WNT11 is associated with time to clinical relapse. It also shows that allele C in rs948006 (WNT11) is protective for clinical relapse.

FIG. 13 shows that gene SLC10A1 is associated with time to virological relapse. It also shows that genotype rs2296651 A/G (SLC10A1) is protective for virological relapse.

FIG. 14 shows that gene TP73 is associated with time to virological relapse. It also shows that allele C rs117634357 (TP73) is protective for virological relapse.

FIG. 15 shows that gene CASZ1 is associated with time to virological relapse. It also shows that allele A rs7534054 (CASZ1) is protective for virological relapse.

FIG. 16 shows that gene ATXN1 is associated with time to virological relapse. It also shows that genotype G/G rs180001 (ATXN1) is protective for virological relapse.

FIG. 17 shows that gene FUT8 (also known as involved in HBV entry into hepatocyte) is associated with time to virological relapse. It also shows that allele G rs2154237 (FUT8) is protective for virological relapse.

FIG. 18 shows that HLA-C region is associated with time to virological relapse. It also shows that genotype rs2394952 A/A (HLA-C) is protective for virological relapse.

FIG. 19 shows the receiver operating characteristic (ROC) curve analyses for treatment regimen, EOT HBsAg (≥ 100 IU/mL vs < 100 IU/mL), rs46688I8, rs948006, rs2934456, rs77586835, rs75876539, rs8050261, rs1 542951, rs231770, rs9277535, or a combination of these covariates, in predicting clinical relapse (CR) at 6 months after stop of treatment. The legend shows the ROC area under the curve (AUC) statistic for model comparison

FIG. 20 shows the receiver operating characteristic (ROC) curve analyses for treatment regimen, EOT HBsAg (≥ 100 IU/mL vs < 100 IU/mL), rs4668818, rs948006, rs2934456, rs77586835, rs75876539, rs8050261, rs1542951, rs231770, rs9277535, or a combination of these covariates, in predicting clinical relapse (CR) at 1 2 months after stop of treatment. The legend shows the ROC area under the curve (AUC) statistic for model comparison

FIG. 21 shows the receiver operating characteristic (ROC) curve analyses for treatment regimen, EOT HBsAg (≥ 100 IU/mL vs < 100 IU/mL), rs4668818, rs948006, rs2934456, rs77586835, rs75876539, rs8050261, rs1542951, rs231770, rs9277535, or a combination of these covariates, in predicting clinical relapse (CR) at 24 months after stop of treatment. The legend shows the ROC area under the curve (AUC) statistic for model comparison

FIG. 22 shows the receiver operating characteristic (ROC) curve analyses for treatment regimen, EOT HBsAg (≥ 100 IU/mL vs < 100 IU/mL), baseline HBeAg status, all SNPs associated to virological relapse (VR), or a combination of these covariates, in predicting virological relapse (VR) at 3 months after stop of treatment. The legend shows the ROC area under the curve (AUC) statistic for model comparison.

FIG. 23 shows the receiver operating characteristic (ROC) curve analyses for treatment regimen, EOT HBsAg (≥ 100 IU/mL vs < 100 IU/mL), baseline HBeAg status, all SNPs associated to virological relapse (VR), or a combination of these covariates, in predicting virological relapse (VR) at 6 months after stop of treatment. The legend shows the ROC area under the curve (AUC) statistic for model comparison.

FIG. 24 The plots below show the receiver operating characteristic (ROC) curve analyses for treatment regimen, EOT HBsAg (≥ 100 IU/mL vs < 100 IU/mL), baseline HBeAg status, all SNPs associated to virological relapse (VR), or a combination of these covariates, in predicting virological relapse (VR) at 12 months after stop of treatment. The legend shows the ROC area under the curve (AUC) statistic for model comparison.

FIG. 25 The plots below show the receiver operating characteristic (ROC) curve analyses for treatment regimen, EOT HBsAg (≥ 100 IU/mL vs < 100 IU/mL), baseline HBeAg status, all SNPs associated to virological relapse (VR), or a combination of these covariates, in predicting virological relapse (VR) at 24 months after stop of treatment. The legend shows the ROC area under the curve (AUC) statistic for model comparison.

FIG. 26 shows that HLA-C*07 region is associated with time to virological relapse. It also shows that no allele copies of HLA-C*07 region is protective for virological relapse

FIG. 27 shows that SNPs in HLA-C region are associated with time to viral relapse. Each SNP measured is represented by a dot, ordered by their genomic position (x-axis) and p-value of association with time to viral relapse (y-axis)

FIG. 28 shows barplot of the distribution of the 24 candidate SNPS indicating a significant association with sustained clinical response for the 3 models tested (additive = ADD, dominant = DOM, recessive = REC) ordered by significance. The rsid is indicated for each SNP, together with its corresponding probe set ID (ThermoFisher UK Biobank or Asia PMRA). In black is represented the population of patients who experienced either a viral relapse or a clinical relapse during the 2 years follow up period. In grey is represented the population of patients who experienced sustained clinical response.

FIG. 29 shows Receiver Operator Characteristic (ROC) analysis, characterizing sensitivity and specificity of the following markers in predicting sustained clinical response (SCR), 2 years after stopping direct antiviral (NUC) treatment:
HBsAg (AUC: 0.65) (black full line)
HBsAg + SLC10A1 (AUC: 0.71) (light gray round-dot line)
HBsAg + combination (dominant) (AUC: 0.75) (light grey long dash dot line)
HBsAg + FUT8 (AUC: 0.76) (dark grey dash line)
HBsAg + combination (additive) (AUC: 0.8) (medium grey long dash line)
HBsAg + FUT8 + SLC10A1 (AUC: 0.8) (medium grey dash dot line)

FIGS. 30A-B show the virological relapse (FIG. 30A) and clinical relapse (FIG. 30B) in the 186 patients in the clinical cohort.

FIG. 31 shows the ROC curve corresponding to three models including: HBsAg level at the end of treatment and treatment regimen (AUC 0.67, full line), adding on top the six Clinical signature SNPs (AUC 0.86, dotted line), the nine SNPs identified in the univariate analysis (AUC 0.89, dashed line).

FIG. 32 shows the overview of the estimated hazard ratios and corresponding 95% confidence intervals for the Cox proportional hazard regression model including the Clinical signature SNPs in Example 3.

FIG. 33 shows the overview of the estimated hazard ratios and corresponding 95% confidence intervals for the Cox proportional hazard regression model including the Virological signature SNPs.

FIG. 34 shows ROC curve corresponding to the model including HBsAg level at the end of treatment and treatment regimen (AUC 0.69), adding on top the two functional SNPs in SLC10A1 and FUT8 genes (AUC 0.79), the 17 Virological signature SNPs (AUC 0.97), and the 33 SNPs identified in the univariate analysis (AUC 0.98).

FIG. 35 shows the overview of the estimated odds ratios and corresponding 95% confidence intervals for the logistic regression model including the SCR signature SNPs.

FIG. 36 shows the ROC curve corresponding to the model including HBsAg level at the end of treatment (AUC 0.66), adding the six SCR signature SNPs (AUC 0.97), including the 15 SNPs previously associated with sustained clinical response (AUC 0.99).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ± 10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “biomarker” or “marker” as used herein refers generally to a molecule, including a gene, protein, carbohydrate structure, or glycolipid, the expression of which in or on a mammalian tissue or cell or secreted can be detected by known methods (or methods disclosed herein) and is predictive or can be used to predict (or aid prediction) for a mammalian cell’s or tissue’s sensitivity to, and in some embodiments, to predict (or aid prediction) an individual’s responsiveness to treatment regimens. The biomarkers disclosed herein are genes and/or proteins whose presence correlates with the absence of relapse of a HBV DAA treatment such as NUC treatment of a liver disease (e.g., chronic hepatitis B infection).

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

As used herein, a “baseline gene expression” of a gene in a subject refers to the gene expression level of the gene in the subject before the subject is treated for the liver diseases.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

As used herein, “treatment” refers to both therapeutic treatment and prophylactic or preventative measures, wherein the object is to prevent or slow down (lessen) the targeted pathologic condition or disorder. Those in need of treatment include those diagnosed with the disorder as well as those prone to have the disorder (e.g., a genetic predisposition) or those in whom the disorder is to be prevented.

A “single nucleotide polymorphism”, or “SNP”, refers to a single base position in an RNA or DNA molecule (e.g., a polynucleotide), at which different alleles, or alternative nucleotides, exist in a population. The SNP position (interchangeably referred to herein as SNP, SNP site, SNP locus) is usually preceded by and followed by highly conserved sequences of the allele (e.g., sequences that vary in less than 1/100 or 1/1000 members of the populations). An individual can be homozygous or heterozygous for an allele at each SNP position. As known to those skilled in the art, a reference SNP ID number, or “rs” ID, is an identification tag assigned by National Center for Biotechnology Information (NCBI) to a group (or cluster) of SNPs that map to an identical location. These SNP rs IDs are mapped to external resources or databases, including NCBI databases. The SNP rs ID number is noted on the records of these external resources and databases in order to point users back to the original dbSNP records. See, for example, information at www.ncbi.nlm.nih.gov/books/NBK44417/#Content.what_is_a_reference_snp_or_rs_i. The detailed information of a SNP identified by a rs ID is available from databases such as wvw.ncbi.nlm.nih.gov/snp/. All such information about any of the SNPs described herein is incorporated by references in its entirely.

Where there are two, three, or four alternative nucleotide sequences at a polymorphic locus, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g, humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism Individuals who are homozygous with respect to one allele are sometimes predisposed to a different phenotype as compared to individuals who are heterozygous or homozygous with respect to another allele.

In genetic analysis that identities one or more pharmacogenomic biomarkers, samples from individuals having different values in a relevant phenotype often are allelotyped and/or genotyped. The term “allelotype” as used herein refers to a process for determining the allele frequency for a polymorphic variant in pooled DNA samples from cases and controls. By pooling DNA from each group, an allele frequency for each locus in each group is calculated These allele frequencies are then compared to one another.

The term “linkage disequilibrium” or “LD” refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. See e g., U.S. 2008/0299125.

In some embodiments. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD. See e.g., U.S. 2008/0299125.

In some embodiments, for diagnostic purposes, if a particular SNP site is found to be useful for diagnosing, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others.

Furthermore, the physical distance over which LD extends along a chromosome differs between different regions of the genome, and therefore the degree of physical separation between two or more SNP sites necessary for LD to occur can differ between different regions of the genome. See e.g., U.S. 2008/0299125.

A genotype or polymorphic variant may be expressed in terms of a “haplotype,” which as used herein refers to a set of DNA variations, or polymorphisms, that tend to be inherited together. A haplotype can refer to a combination of alleles or to a set of SNPs found on the same chromosome. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “array” or “microarray” refers to an ordered arrangement of hybridizable array elements, preferably polynucleotide probes e.g, oligonucleotides), on a substrate The substrate can be a solid substrate, such as a glass slide, or a semi-solid substrate, such as nitrocellulose membrane.

The term “administering” with respect to the methods of the invention, means a method for therapeutically or prophylactically preventing, treating or ameliorating a syndrome, disorder or disease as described herein. Such methods include administering an effective amount of said therapeutic agent at different times during the course of a therapy or concurrently in a combination form. The methods of the invention are to be understood as embracing all known therapeutic treatment regimens.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., CHB).

The term “HBV direct-acting antiviral agent” or “HBV DAA” is in accordance with its ordinary meaning in the field and includes any agent which directly interacts with, more particularly inhibits, the cell cycle of HBV, e.g., the cell entry (more particularly the hepatocyte entry) of HBV and/or the replication of HBV. Examples of HBV DAAs include, but not limited to, nucleotides or nucleosides (NUCs), entry inhibitors, covalently closed circular DNA (cccDNA) inhibitors, transcription inhibitors, RNA silencers, HBV capsid inhibitors, and HBsAg release inhibitors.

The term “non-DAA treatment” encompasses a treatment using non-DAA agents, a treatment using a combination of a DAA agent and other agent, as well as stopping treatment.

The term “non-NUC treatment” encompasses a treatment using non-NUC agents, a treatment using a combination of a NUC agent and other agent, as well as stopping treatment.

The term “p-value” is intended in accordance with its ordinary meaning in the field. For example, the p-value is used in the context of null hypothesis to quantify the statistical significance of an observed result, assuming that the null hypothesis is correct. It measures the probability of the observation. The lower the p-value, the greater the statistical significance of the observation, e.g., the less likely that it is due to simple random chance. For example, a p-value of 0.05 signifies a 5% probability that the observation is by random chance, while a p-value of 1.0E-05 signifies a 0.001% probability that the observation is by random chance. In a large-scale multiple testing, multiple testing correction adjusts the individual p-value to keep the overall error rate (false positive rate) to less than or equal to a desirable level. When multiple testing correction is to be taken into account, a SNP has to be associated with a lower p-value to reach significant level. Multiple testing correction limits the risk of false positive when multiple hypothesis are tested.

### Treatment of CHB

Clearance of HBsAg, with seroconversion to HBs antibodies (anti-HBs) is the closest correlate of cure and the ultimate goal of CHB therapy. CHB treatments include immunomodulators and HBV direct-acting antiviral agents (DAAs) such as NUCs. Examples of immunomodulators include, but are not limited to, IFN-α, Peg-IFN-α, thymosin-α1 and oxymatrine. Interferons (IFNs) are cytokines which interfere with viral replication in host cells by inhibiting viral DNA synthesis, and enhancing the cellular immune response against HBV-infected hepatocytes. In general, interferon (IFN) therapy has a finite duration of treatment and is more likely to produce a sustained virological response. Its use, however, is limited by high costs and numerous associated side effects.

Examples of NUCs include, but are not limited to tenofovir, entecavir, lamivudine, adefovir, and telbivudine. The goal of NUC therapy (NA) for chronic hepatitis B (CHB) is to suppress hepatitis B virus (HBV) replication in a sustained manner, preventing disease progression to decompensated cirrhosis and hepatocellular carcinoma (HCC). Overall, all NAs have an excellent safety profile across a wide spectrum of persons with CHB, including those with decompensated cirrhosis and transplant recipients. Indeed, more than 90% of patients with CHB are currently treated with oral NUCs worldwide. However, the NUCs tend not to eradicate HBV as they do not impact HBV cccDNA, which acts as an ongoing source of viral persistence, therefore, long-term treatment with NUCs is required to maintain virological control.

To avoid lifelong NUC treatment, new strategies are being assessed in clinical trials, including switching to or adding on Peg-IFN, combination with oral immunomodulatory agents, and discontinuation in selected HBeAg-negative patients according to HBsAg levels.

### Relapses After HBV DAA Treatment of CHB

The duration of HBV DAA treatment requires that at least once complete virologic suppression is achieved. Although loss of HBsAg is the ideal endpoint associated with sustained off-treatment virologic suppression, HBsAg is only cleared in a minority of CHB patients after antiviral therapy. HBeAg loss and/or seroconversion has been widely used as a surrogate endpoint of CHB therapy, and several practice guidelines suggest that HBV DCC such as NUC treatment may be stopped when the patient achieves HBV DNA < 60 IU/mL, ALT < 80 U/L, and HBeAg negative. Nonetheless, approximately 25% to 50% of the patients may still develop relapse after stopping anti-viral therapy even if these recommendations are followed.

Hepatitis relapses involve transient abnormalities in the alanine aminotransferase (ALT) level or the HBV DNA level, as well as HBeAg level. Hepatitis relapses can be characterized as virological relapse, biomedical relapse, or clinical relapse. Currently, using the same limits as the guidelines for the initiation of therapy, an HBV DNA level ≥2000 IU/mL or HBeAg positive can be considered as virological relapse. Biochemical relapse is defined as an elevation of ALT levels >1 time (1x), 1.5x or 2x the upper limit of normal (ULN) depending on study criteria. The current upper limit of serum ALT, though varied among laboratories, is generally around 40 IU/L. In some studies, the term clinical relapse is used, which considers group both virological and biochemical relapses. In some studies, a patient is denoted a sustained clinical responder in case no clinical and no virological relapse occurred during the entire follow-up period after treatment cessation.

### Biomarker Panel and Probes for Predicting a Relapse or a Sustained Clinical Response after HBV DAA Treatment of CHB

The present invention relates generally to the prediction of a relapse or a sustained clinical response in a subject diagnosed with CHB after treatment, and provides methods, reagents, and kits useful for this purpose. Provided herein are biomarkers that are indicative of and/or predictive for a relapse or a sustained clinical response after the treatment. In certain embodiments, the treatment is a HBV DAA treatment such as a NUC treatment. In certain embodiments, the present invention provides a panel of SNPs for predicting a relapse after the discontinuation of a HBV DAA treatment in a subject diagnosed with CHB. According to the embodiments of this invention, the presence of the panel is negatively associated with the incidence of such relapse. In certain embodiments, the present invention provides a panel of SNPs for predicting a sustained clinical response after the discontinuation of HBV DAA treatment in a subject diagnosed with CHB. According to the embodiments of this invention, the presence of the panel is positively associated with the incidence of such sustained clinical response.

Also provided are kits, chips, devices, or assays for use in accordance with the present invention Such an assay, chip, device, or a kit can comprise a plurality of primers or probes to detect genetic signature of SNPs described herein. Such kits, chips, devices can include instruments and instructions that a subject can use to obtain a sample, e.g., of buccal cells or blood, without the aid of a health care provider.

In some embodiments, the invention provides compositions and kits comprising primers and primer pairs, which allow the specific amplification of the polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers can be used to detect the presence of polynucleotides in a sample and as a means for detecting cell expressing proteins encoded by the polynucleotides. As will be understood by the skilled artisan, a great many different primers and probes can be prepared based on the sequences provided herein and used effectively to amplify, clone and/or determine the presence of a SNP of interest.

The application also contemplates the development of computer algorithm which will convert the test results generated from the measurement of the genomic biomarkers into an output, e.g., a score, which will be used to determine in whether an individual is likely to have a relapse or a sustained clinical response after a treatment of CHB.

In one general aspect, provided is an isolated set of probes capable of detecting a panel of SNPs comprising one or more SNPs selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 of the SNPs described herein. The set of probes can be used to predict the probability of an individual to have a relapse after a treatment of CHB, preferably a HBV DVV treatment, more preferably a NUC treatment, or can be used to predict the sustained clinical response after a treatment of CHB, preferably a HBV DAA treatment, more preferably a NUC treatment. More preferably, the panel of SNPs contains at least two SNPs described herein.

In certain embodiment, the one or more SNPs are associated with time to relapse.

In certain embodiment, the one or more SNPs are associated with the time to relapse with a p-value of less than 0.05, preferably less than 1.0E-05, more preferably 5.40E-06 or less.

In certain embodiment, the one or more SNPs are associated with the time to relapse with a p-value of less than 0.05, and the one or more SNPs are selected from the group consisting of rs231770, rs9277535, rs3130542, rs7574865, rs2296651, and rs1419881, or a complementary sequence thereof.

In certain embodiment, the one or more SNPs are associated with the time to relapse with a p-value of 0.001 to 0.05, and the one or more SNPs are selected from the group consisting of rs231770, rs9277535, rs7574865, rs2296651, and rs1419881, or a complementary sequence thereof.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542, or a complementary sequence thereof.

In certain embodiment, the isolated set of probes is capable of detecting a panel of SNPs comprising one or more SNPs selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, and rs1542951, or a complementary sequence thereof. In further embodiments, the one or more SNPS are associated with time to relapse with a p-value of less than 0.05, preferably less than 1.0E-05, more preferably 5.40E-06 or less. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 of the SNPs described herein. The set of probes can be used to predict the probability of an individual to have a virological relapse or a clinical relapse after a treatment of CHB.

In certain embodiment, the isolated set of probes is capable of detecting a panel of SNPs comprising one or more SNPs selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof. The panel of SNPs can contain 1, 2, 3, 4, 5, 6, 7, 8, 9, 10, 11, 12, 13, 14, 15, 16, 17, 18, 19, 20, 21, 22, or 23 of the SNPs described herein. In further embodiments, the one or more SNPs are associate with time to relapse with a p-value of less than 0.05, preferably less than 1.0E-05, more preferably 5.40E-06 or less. The set of probes can be used to predict the probability of an individual to have a virological relapse or a clinical relapse after a treatment of CHB.

In further embodiments, the isolated set of probes is capable of detecting a panel of alleles comprising one or more alleles selected from the group consisting of allele T in rs4668818, allele C in rs948006, allele A in rs2934456, allele C in rs75876539, allele G in rs8050261, allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, and allele A in rs1419881, or a complementary sequence thereof. The alleles described herein are protective against virological relapse or clinical relapse. The panel of alleles can contain 1, 2, 3, 4, 5, 6, 7, 8, 9, 10, 11, 12, 13, 14, 15, 16, 17, 18, 19, 20, 21, 22, or 23 of the alleles described herein. The set of probes can be used to predict the probability of an individual to have a virological relapse or a clinical relapse after a treatment of CHB.

In further embodiments, the isolated set of probes is capable of detecting a panel of alleles comprising one or more alleles selected from the group consisting of allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, and allele G in rs1419881, or a complementary sequence thereof. The alleles described herein are predictive of virological relapse or clinical relapse. The panel of alleles can contain 1, 2, 3, 4, 5, 6, 7, 8, 9, 10, 11, 12, 13, 14, 15, 16, 17, 18, 19, 20, 21, 22, or 23 of the alleles described herein. The set of probes can be used to predict the probability of an individual to have a virological relapse or a clinical relapse after a treatment of CHB.

In certain embodiment, the isolated set of probes is capable of detecting a panel of SNPs comprising one or more SNPs selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 of the SNPs described herein. In further embodiments, the one or more SNPs are associate with time to relapse with a p-value of less than 0.05, preferably less than 1.0E-05, more preferably 5.40E-06 or less. The set of probes can be used to predict the probability of an individual to have a virological relapse after a treatment of CHB.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518 and rs3130542, or a complementary sequence thereof.

In certain embodiment, the isolated set of probes is capable of detecting a panel of SNPs comprising one or more SNPs selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the SNPs described herein. In further embodiments, the one or more SNPs are associate with time to relapse with a p-value of less than 0.05, preferably less than 1.0E-05, more preferably 5.40E-06 or less. The set of probes can be used to predict the probability of an individual to have a virological relapse after a treatment of CHB.

In further embodiments, the isolated set of probes is capable of detecting a panel of alleles comprising one or more alleles selected from the group consisting of allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, and allele C in rs10235518, or a complementary sequence thereof. The alleles described herein are protective against virological relapse. The panel of alleles can contain 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the alleles described herein. The set of probes can be used to predict the probability of an individual to have a virological relapse after a treatment of CHB.

In certain embodiment, the isolated set of probes is capable of detecting a panel of SNPs comprising one or more SNPs selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof. The panel of SNPs can contain 1, 2, 3, 4, 5, 6, 7, 8, 9, 10, 11, 12, 13, 14, 15, 16, or 17, of the SNPs described herein. In further embodiments, the one or more SNPs are associate with time to relapse with a p-value of less than 0.05. The set of probes can be used to predict the probability of an individual to have a virological relapse after a treatment of CHB.

In further embodiments, the one or more SNPs are associate with time to relapse with a p-value of less than 0.05, preferably less than 1.0E-05, more preferably 5.40E-06 or less, and the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, and rs10235518.

In certain embodiment, the isolated set of probes is capable of detecting a panel of SNPs comprising one or more SNPs selected from the group consisting of rs4668818, rs948006, rs2934456, rs77586835, rs75876539, rs8050261, rs1542951, rs231770, and rs9277535, or a complementary sequence thereof. The panel of SNPs can contain 1, 2, 3, 4, 5, 6, 7, 8 or 9 of the SNPs described herein. In further embodiments, the one or more SNPs are associate with time to relapse with a p-value of less than 0.05, preferably less than 1.0E-05, more preferably 5.40E-06 or less. The set of probes can be used to predict the probability of an individual to have a clinical relapse after a treatment of CHB.

In certain embodiment, the isolated set of probes is capable of detecting a panel of SNPs comprising one or more SNPs selected from the group consisting of rs4668818, rs948006, rs2934456, rs77586835, rs75876539, rs8050261, and rs1542951, or a complementary sequence thereof. The panel of SNPs can contain 1, 2, 3, 4, 5, 6 or 7 of the SNPs described herein. In further embodiments, the one or more SNPs are associate with time to relapse with a p-value of less than 0.05, preferably less than 1.0E-05, more preferably 5.40E-06 or less. The set of probes can be used to predict the probability of an individual to have a clinical relapse after a treatment of CHB.

In further embodiments, the isolated set of probes are capable of detecting a panel of alleles comprising one or more alleles selected from the group consisting of allele T in rs4668818, allele C in rs948006, allele A in rs2934456, allele T in rs77586835, allele C in rs75876539, allele G in rs8050261, and allele A in rs1542951, or a complementary sequence thereof. The alleles described herein are protective against clinical relapse. The panel of alleles can contain 1, 2, 3, 4, 5, 6 or 7 of the alleles described herein. The set of probes can be used to predict the probability of an individual to have a clinical relapse after a treatment of CHB.

In certain embodiment, the isolated set of probes is capable of detecting a panel of SNPs comprising one or more SNPs selected from the group consisting of rs4668818, rs948006, rs2934456, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, or 6 of the SNPs described herein. The set of probes can be used to predict the probability of an individual to have a clinical relapse after a treatment of CHB.

In one embodiment, the SNP is rs2296651 or a complementary sequence thereof.

In one embodiment, the SNP is rs231770 or a complementary sequence thereof .

In certain embodiment, the isolated set of probes is capable of detecting a panel of SNPs comprising one or more SNPs selected from the group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof. The panel of SNPs can contain 1, 2, 3, 4, 5, 6, 7, 8, 9, 10, 11, 12, 13, 14, 15, 16, 17, 18, 19, 20, 21, 22, 23 or 24 of the SNPs described herein. In further embodiments, the one or more SNPs are associate with time to relapse with a p-value of less than 0.05. The set of probes can be used to predict the probability of an individual to have a sustained clinical response after a treatment of CHB.

In certain embodiment, the isolated set of probes is capable of detecting a panel of alleles comprising one or more alleles selected from the group consisting of allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, and allele T in rs7459445, or a complementary sequence thereof. The alleles described herein are predictive of sustained clinical response. The panel of alleles can contain 1, 2, 3, 4, 5, 6, 7, 8, 9, 10, 11, 12, 13, 14, 15, 16, 17, 18, 19, 20, 21, 22, 23 or 24 of the alleles described herein. The set of probes can be used to predict the probability of an individual to have a sustained clinical response after a treatment of CHB.

In certain embodiment, the isolated set of probes is capable of detecting a panel of alleles comprising one or more alleles SNPs selected from the group consisting of allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, and allele C in rs7459445, or a complementary sequence thereof. The alleles described herein are predictive of no sustained clinical response. The panel of alleles can contain 1, 2, 3, 4, 5, 6, 7, 8, 9, 10, 11, 12, 13, 14, 15, 16, 17, 18, 19, 20, 21, 22, 23 or 24 of the alleles described herein. The set of probes can be used to predict the probability of an individual to have no sustained clinical response after a treatment of CHB.

In certain embodiment, the isolated set of probes is capable of detecting a panel of SNPs comprising one or more SNPs selected from the group consisting of rs4315565, rs2154237, rs9828024, rs12105972, rs3943102 and rs2296651, or a complementary sequence thereof. In further embodiments, the one or more SNPs are associate with time to relapse with a p-value of less than 0.05, preferably less than 0.01. In further embodiments, the one or more SNPs are associate with time to relapse with a p-value of less than 0.05, preferably less than 0.01. The panel of SNPs can contain 1, 2, 3, 4, 5, or 6 of the SNPs described herein. The set of probes can be used to predict the probability of an individual to have a sustained clinical response after a treatment of CHB.

In further embodiments, the isolated set of probes is capable of detecting a panel of SNPs comprising one or more SNPs selected from the group consisting of rs2154237 and rs2296651, or a complementary sequence thereof. In further embodiments, the one or more SNPs are associate with time to relapse with a p-value of less than 0.05, preferably less than 0.01. The panel of SNPs can contain 1 or 2 of the SNPs described herein. The set of probes can be used to predict the probability of an individual to have a sustained clinical response after a treatment of CHB.

In another general aspect, provided is an isolated set of probes for use in treating a chronic hepatitis B (CHB) infection in a subject in need thereof, wherein the set of probes detects a panel of SNPs or a complementary sequence thereof, and the panel comprises one or more SNPs associated with time to relapse, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof.

In certain embodiment, the one or more SNPs are associated with the time to relapse with a p-value of less than 0.05, preferably less than 1.0E-05, more preferably 5.40E-06 or less.

In certain embodiment, the one or more SNPs are associated with the time to relapse with a p-value of less than 0.05, and the one or more SNPs are selected from the group consisting of rs231770, rs9277535, rs3130542, rs7574865, rs2296651, and rs1419881, or a complementary sequence thereof.

In certain embodiment, the one or more SNPs are associated with the time to relapse with a p-value of 0.001 to 0.05, and the one or more SNPs are selected from the group consisting of rs231770, rs9277535, rs7574865, rs2296651, and rs1419881, or a complementary sequence thereof.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less.

In certain embodiment, the one or more SNPs are selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof. In further embodiments, the one or more SNPs are associate with time to relapse with a p-value of less than 0.05, preferably less than 1.0E-05, more preferably 5.40E-06 or less.

In further embodiments, the one or more SNPs comprise allele T in rs4668818, allele C in rs948006, allele A in rs2934456, allele C in rs75876539, allele G in rs8050261, and allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, or allele A in rs1419881, or a complementary sequence thereof.

In further embodiments, the one or more SNPs comprise allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, or allele G in rs1419881, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof. In further embodiments, the one or more SNPs are associate with time to relapse with a p-value of less than 0.05, preferably less than 1.0E-05, more preferably 5.40E-06 or less.

In certain embodiment, the one or more SNPs are associated with time to relapse with a p-value of 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518 and rs3130542, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof. In further embodiments, the one or more SNPs are associate with time to relapse with a p-value of 5.40E-06 or less.

In further embodiments, the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, or allele C in rs10235518, or a complementary sequence thereof.

In certain embodiment, the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof. In further embodiments, the one or more SNPs are associate with time to relapse with a p-value of less than 0.05.

In further embodiments, the one or more SNPs are associate with time to relapse with a p-value of less than 0.05, preferably less than 1.0E-05, more preferably 5.40E-06 or less, and the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, and rs10235518, or a complementary sequence thereof.

In certain embodiment, the one or more SNPs are selected from the group consisting of rs4668818, rs948006, rs2934456, rs77586835, rs75876539, rs8050261, rs1542951, rs231770, and rs9277535, or a complementary sequence thereof. In further embodiments, the one or more SNPs are associate with time to relapse with a p-value of less than 0.05, preferably less than 1.0E-05, more preferably 5.40E-06 or less.

In certain embodiment, the one or more SNPs are selected from the group consisting of rs4668818, rs948006, rs2934456, rs77586835, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less.

In further embodiments, the one or more SNPs comprise allele T in rs4668818, allele C in rs948006, allele A in rs2934456, allele T in rs77586835, allele C in rs75876539, allele G in rs8050261, or allele A in rs1542951, or a complementary sequence thereof.

In certain embodiment, the one or more SNPs are selected from the group consisting of rs4668818, rs948006, rs2934456, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less.

In one embodiment, the SNP is rs2296651 or a complementary sequence thereof.

In one embodiment, the SNP is rs231770 or a complementary sequence thereof.

In certain embodiment, the one or more SNPs are selected from the group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof. In further embodiments, the one or more SNPs are associate with time to relapse with a p-value of less than 0.05.

In certain embodiment, the one or more SNPs comprise allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, or allele T in rs7459445, or a complementary sequence thereof.

In certain embodiment, the one or more SNPs comprise allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, or allele C in rs7459445, or a complementary sequence thereof.

In certain embodiment, the one or more SNPs are selected from the group consisting of rs4315565, rs2154237, rs9828024, rs12105972, rs3943102 and rs2296651, or a complementary sequence thereof. In further embodiments, the one or more SNPs are associate with time to relapse with a p-value of less than 0.05, preferably less than 0.01.

In further embodiments, the one or more SNPs selected from the group consisting of rs2154237 and rs2296651, or a complementary sequence thereof. In further embodiments, the one or more SNPs are associate with time to relapse with a p-value of less than 0.05, preferably less than 0.01.

In certain embodiments, the treatment further comprises measuring the level of at least one of HBV DNA, alanine aminotransferase (ALT), and hepatitis B e-antigen (HBeAg) in a biological sample of the subject.

In certain embodiments, if the panel of the one or more SNPs is detected in the biological sample, the treatment comprises:
(1) treating the subject with a therapeutically effective amount of a HBV direct-acting antiviral agent (DAA) two years or later after the discontinuation of the HBV DAA treatment,
(2) continuing treating the subject with the HBV DAA until CHB infection is suppressed in the subject; or
(3) monitoring relapse in the subject two years or later after the discontinuation of the HBV DAA treatment.

In certain embodiments, if none of the one or more SNPs is detected in the biological sample, the treatment comprises:
(1) monitoring relapse in the subject prior to two years after the discontinuation of the HBV DAA treatment;
(2) administering to the subject a therapeutically effective amount of a non-DAA agent; or
(3) switching from the HBV DAA treatment to a non-DAA treatment.

In certain embodiments, the HBV DAA treatment is a NUC treatment. The NUC can be tenofovir, entecavir, lamivudine, adefovir, or telbivudine.

In certain embodiments, the non-DAA treatment is a non-NUC agent, such as interferon.

In certain embodiments, the subject discontinues the HBV-DAA treatment when the subject achieves at least one of HBV DNA < 60 IU/mL, alanine aminotransferase (ALT) < 80 U/L, and hepatitis B e-antigen (HBeAg) negative.

In certain embodiments, the subject further achieves HBsAg < 100 IU/mL at the time of discontinuation of the HBV-DAA treatment.

In certain embodiments, the subject has no virological relapse at or after 3 months, 6 months, 12 months, 18 months, 24 months, or 36 months after the discontinuation of the HBV-DAA treatment, or anytime in between, and the virological relapse is identified as HBV DNA ≥ 2000 IU/ml or HBeAg positive.

In certain embodiments, the subject has no clinical relapse at or after 3 months, 6 months, 12 months, 18 months, 24 months, or 36 months after the discontinuation of the HBV DAA treatment, or anytime in between, and the clinical relapse is identified as i) HBV DNA ≥ 2000 IU/ml or HBeAg positive, and ii) ALT ≥ 80 U/L.

In certain embodiments, the biological sample is a tissue sample, a cellular sample, or a blood sample.

In certain embodiments, the probes are arranged in an array, such as a microarray.

In some embodiments, an array of the application comprises individual or collections of nucleic acid molecules useful for detecting SNPs described herein. For instance, an array of the application can comprise a series of discretely placed individual nucleic acid oligonucleotides or sets of nucleic acid oligonucleotide combinations that are hybridizable to a sample comprising nucleic acids having a target SNP, whereby such hybridization is indicative of the presence of the target SNP.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into nucleic acid molecules that are synthesized. The synthesized product is then contacted with a solid substrate, such as a glass slide, which is coated with an aldehyde or another reactive group which will form a covalent link with the reactive group that is on the amplified product and become covalently attached to the glass slide. Other methods, such as those using amino propryl silica surface chemistry, are also known in the art, as disclosed at world wide web at cmt.corning.com and cmgm.stanford.edu/pbrownl

Attachment of groups to oligonucleotides which could be later converted to reactive groups is also possible using methods known in the art. Any attachment to nucleotides of oligonucleotides will become part of oligonucleotide, which could then be attached to the solid surface of the microarray. Amplified nucleic acids can be further modified, such as through cleavage into fragments or by attachment of detectable labels, prior to or following attachment to the solid substrate, as required and/or permitted by the techniques used

For use in the applications described or suggested above, kits or articles of manufacture are also provided. Such kits can comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be a polynucleotide specific for a polynucleotide comprising a SNP described herein. Where the kit utilizes nucleic acid hybridization to detect the target nucleic acid, the kit can also have containers containing nucleotide(s) for amplification of the target nucleic acid sequence and/or a container comprising a reporter means, such as a biotin-binding protein, such as avidin or streptavidin, bound to a reporter molecule, such as an enzymatic, fluorescent, or radioisotope label.

Kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

Other optional components in the kit include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc. An additional component is an enzyme, for example, including but not limited to, a nuclease, a ligase, or a polymerase.

SNPs are the most common type of genetic variation among people. Each SNP represents a difference in a single DNA building block, called a nucleotide. For example, a SNP may replace the nucleotide cytosine (C) with the nucleotide thymine (T) in a certain stretch of DNA. These variations may be unique or occur in many individuals. Most commonly, these variations are found in the DNA between genes.

In certain embodiments, the present invention provides a panel of SNPs that indicate the subject will have a relapse or not to the treatment of CHB. Preferably, the treatment of CHB is a HBV DAA treatment. More preferably, the HBV DAA treatment is a NUC treatment.

The panel of SNPs is able to identify subsets of patients with different risk to hepatitis relapses after treatment of CHB, which could be beneficial in many ways, including reduced exposure of patients to ineffective treatments, achievement of higher response rates, and the ability to treat predicted patients with alternative therapies to avoid or minimize possible relapse. The panel of biomarkers can additionally be used for other purposes, such as to stratify patients in clinical trials, reduce sample size in proof of concept trials by excluding subpopulations, and balance treatment arms in clinical trials.

The measurement of genetic variations of SNPs between members of a species is called SNP genotyping. It is a form of genotyping, which is the measurement of more general genetic variation.

Variations can be detected by any methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom.

Variations can also be detected by mismatch detection methods. Mismatches are hybridized nucleic acid duplexes which are not 100% complementary. The lack of total complementarity may be due to deletions, insertions, inversions, or substitutions.

In certain embodiments, the SNPS or alleles are determined by Genome-wide genotyping (GMAS), wherein the genotype calling is performed on biallelic SNPs. In certain embodiments, the SNPs or alleles are determined by Human leukocyte antigen (HLA) typing by Sanger sequencing.

Detailed information on SNPs can be available from Single Nucleotide Polymorphism Database (dbSNP), a free public archive for genetic variation within and across different species developed and hosted by the National Center for Biotechnology Information (NCBI) in collaboration with the National Human Genome Research Institute (NHGRI). In dbSNP, a SNP is identified by a reference SNP ID number (“rs#”).

### Methods of Use

Provided herein are methods of predicting a relapse or a sustained clinical response after discontinuation of a treatment in a chronic hepatitis B (CHB) infection in a subject in need thereof using a panel of SNPs according to an embodiment of the invention. The panel of SNPs can also be used for other purposes, such as to stratify patients in clinical trials, reduce sample size in proof of concept trials by excluding subpopulations, and balance treatment arms in clinical trials.

In one general aspect, the application provides a method of predicting a relapse or a sustained clinical response of a subject undergoing or completed a treatment of a chronic hepatitis B (CHB) infection, the method comprising: obtaining a biological sample from the subject; and detecting in the biological sample the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, s2934456, rs77586835, rs75876539, rs8050261, rs1542951, rs231770, rs9277535, rs3130542, rs7574865, rs2296651, and rs1419881, or a complementary sequence thereof, wherein the presence of the panel of the one or more of the SNPs predicts no relapse or less likely to have relapse in the subject, and the absence of all of the SNPs predicts relapse in the subject. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 of the SNPs described herein. Preferably, the panel of SNPs contains at least two SNPs described herein.

In certain embodiments, the method of predicting a relapse or a sustained clinical response is an in vitro method.

In further embodiments, the vitro method can be used to monitor relapse of a chronic hepatitis B (CHB) infection in a subject, wherein the method further comprises: monitoring the relapse in the subject two years or later after the discontinuation of the NUC treatment, if the panel of the one or more SNPs is detected in the biological sample; or monitoring the relapse in the subject prior to two years after the discontinuation of the NUC treatment, if none of the one or more SNPs is detected in the biological sample.

In certain embodiment, the one or more SNPs are associated with time to relapse.

In certain embodiment, the one or more SNPs are associated with the time to relapse with a p-value of less than 0.05, preferably less than 1.0E-05, more preferably 5.40E-06 or less.

In certain embodiment, the one or more SNPs are associated with the time to relapse with a p-value of less than 0.05, and the one or more SNPs are selected from the group consisting of rs231770, rs9277535, rs3130542, rs7574865, rs2296651, and rs1419881.

In certain embodiment, the one or more SNPs are associated with the time to relapse with a p-value of 0.001 to 0.05, and the one or more SNPs are selected from the group consisting of rs231770, rs9277535, rs7574865, rs2296651, and rs1419881.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542.

In certain embodiment, the method comprises detecting in the biological sample the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, and rs1542951, or a complementary sequence thereof, wherein the presence of the panel of the one or more of the SNPs predicts no relapse or less likely to have relapse in the subject, and the absence of all of the SNPs predicts relapse in the subject. In further embodiments, the one or more SNPS are associated with time to relapse with a p-value of less than 0.05, preferably less than 1.0E-05, more preferably 5.40E-06 or less. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 of the SNPs described herein.

In certain embodiment, the method comprises detecting in the biological sample the presence of a panel of one or more single nucleotide polymorphisms (SNPs) selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof, wherein the presence of the panel of the one or more of the SNPs predicts no relapse or less likely to have relapse in the subject, and the absence of all of the SNPs predicts relapse in the subject. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, 6, 7, 8, 9, 10, 11, 12, 13, 14, 15, 16, 17, 18, 19, 20, 21, 22 or 23 of the SNPs described herein.

In certain embodiments, the method further comprises detecting in the biological sample the presence of a panel of one or more alleles selected from the group consisting of allele T in rs4668818, allele C in rs948006, allele A in rs2934456, allele C in rs75876539, allele G in rs8050261, and allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, and allele A in rs1419881, or a complementary sequence thereof, wherein the presence of the panel of the one or more of the alleles predicts no relapse or less likely to have a relapse in the subject, and the absence of all of the alleles predicts a relapse in the subject. The panel of alleles can contain 1, 2, 3, 4, 5, 6, 7, 8, 9, 10, 11, 12, 13, 14, 15, 16, 17, 18, 19, 20, 21, 22, or 23 of the alleles described herein.

In certain embodiments, the method further comprises detecting in the biological sample the presence of a panel of one or more alleles selected from the group consisting of allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, and allele G in rs1419881, or a complementary sequence thereof, wherein the presence of the panel of the one or more of the alleles predicts a relapse in the subject, and the absence of all of the alleles predicts no relapse or less likely to have a relapse in the subject. The panel of alleles can contain 1, 2, 3, 4, 5, 6, 7, 8, 9, 10, 11, 12, 13, 14, 15, 16, 17, 18, 19, 20, 21, 22, or 23 of the alleles described herein.

In certain embodiment, the method comprises detecting in the biological sample the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof, wherein the presence of the panel of the one or more of the SNPs predicts no virological relapse or less likely to have virological relapse in the subject, and the absence of all of the SNPs predicts virological relapse in the subject. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 of the SNPs described herein.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518 and rs3130542 .

In certain embodiment, the method comprises detecting in the biological sample the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof, wherein the presence of the panel of the one or more of the SNPs predicts no virological relapse or less likely to have virological relapse in the subject, and the absence of all of the SNPs predicts virological relapse in the subject. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the SNPs described herein.

In certain embodiments, the method further comprises detecting in the biological sample the presence of a panel of one or more alleles selected from the group consisting of allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, and allele C in rs10235518, or a complementary sequence thereof, wherein the presence of the panel of the one or more of the alleles predicts no virological relapse or less likely to have virological relapse in the subject, and the absence of all of the alleles predicts a virological relapse in the subject. The panel of alleles can contain 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the alleles described herein.

In certain embodiment, the method comprises detecting in the biological sample the presence of a panel of one or more single nucleotide polymorphisms (SNPs) selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof, wherein the presence of the panel of the one or more of the SNPs predicts no virological relapse or less likely to have virological relapse in the subject, and the absence of all of the SNPs predicts virological relapse in the subject. In further embodiments, the one or more SNPs are associate with time to relapse with a p-value of less than 0.05. The panel of SNPs can contain 1, 2, 3, 4, 5, 6, 7, 8, 9, 10, 11, 12, 13, 14, 15, 16, or 17, of the SNPs described herein.

In further embodiments, the one or more SNPs are associate with time to relapse with a p-value of less than 0.05, preferably less than 1.0E-05, more preferably 5.40E-06 or less, and the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, and rs10235518.

In certain embodiment, the method comprises detecting in the biological sample the presence of a panel of one or more single nucleotide polymorphisms (SNPs) selected from the group consisting of rs4668818, rs948006, rs2934456, rs77586835, rs75876539, rs8050261, rs1542951, rs231770, and rs9277535, or a complementary sequence thereof, wherein the presence of the panel of the one or more of the SNPs predicts no clinical relapse or less likely to have clinical relapse in the subject, and the absence of all of the SNPs predicts clinical relapse in the subject. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, 6, 7, 8 or 9 of the SNPs described herein.

In certain embodiment, the method comprises detecting in the biological sample the presence of a panel of one or more single nucleotide polymorphisms (SNPs) selected from the group consisting of rs4668818, rs948006, rs2934456, rs77586835, rs75876539, rs8050261, and rs1542951, or a complementary sequence thereof, wherein the presence of the panel of the one or more of the SNPs predicts no clinical relapse or less likely to have clinical relapse in the subject, and the absence of all of the SNPs predicts clinical relapse in the subject.. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, 6 or 7 of the SNPs described herein.

In certain embodiments, the method further comprises detecting in the biological sample the presence of a panel of one or more alleles selected from the group consisting of allele T in rs4668818, allele C in rs948006, allele A in rs2934456, allele T in rs77586835, allele C in rs75876539, allele G in rs8050261, and allele A in rs1542951, or a complementary sequence thereof, wherein the presence of the panel of the one or more of the alleles predicts no clinical relapse or less likely to have clinical relapse in the subject, and the absence of all of the alleles predicts clinical relapse in the subject. The panel of SNPs can contain 1, 2, 3, 4, 5, 6 or 7 of the alleles described herein.

In certain embodiment, the method comprises detecting in the biological sample the presence of a panel of one or more single nucleotide polymorphisms (SNPs) selected from the group consisting of rs4668818, rs948006, rs2934456, rs75876539, rs8050261, and rs1542951, wherein the presence of the panel of the one or more of the SNPs predicts no clinical relapse or less likely to have clinical relapse in the subject, and the absence of all of the SNPs predicts clinical relapse in the subject.. In further embodiments, the one or more SNPs are associate with time to relapse with a p-value of less than 0.05, preferably less than 1.0E-05, more preferably 5.40E-06 or less. The panel of SNPs can contain 1, 2, 3, 4, 5, or 6 of the SNPs described herein.

In one embodiment, the SNP is rs2296651.

In one embodiment, the SNP is rs231770.

In certain embodiment, the method comprises detecting in the biological sample the presence of a panel of one or more single nucleotide polymorphisms (SNPs) selected from group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof, wherein the presence of the panel of the one or more of the SNPs predicts sustained clinical response in the subject, and the absence of all of the SNPs predicts a relapse in the subject. In further embodiments, the one or more SNPs are associate with time to relapse with a p-value of less than 0.05. The panel of SNPs can contain 1, 2, 3, 4, 5, 6, 7, 8, 9, 10, 11, 12, 13, 14, 15, 16, 17, 18, 19, 20, 21, 22, 23 or 24 of the SNPs described herein.

In certain embodiments, the method further comprises detecting in the biological sample the presence of a panel of one or more alleles selected from the group consisting of allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, and allele T in rs7459445, or a complementary sequence thereof, wherein the presence of the panel of the one or more of the alleles predicts sustained clinical response in the subject. The panel of alleles can contain 1, 2, 3, 4, 5, 6, 7, 8, 9, 10, 11, 12, 13, 14, 15, 16, 17, 18, 19, 20, 21, 22, 23, or 24 of the alleles described herein.

In certain embodiments, the method further comprises detecting in the biological sample the presence of a panel of one or more alleles selected from the group consisting of allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, and allele C in rs7459445, or a complementary sequence thereof, wherein the presence of the panel of the one or more of the alleles predicts no sustained clinical response likely in the subject. The panel of alleles can contain 1, 2, 3, 4, 5, 6, 7, 8, 9, 10, 11, 12, 13, 14, 15, 16, 17, 18, 19, 20, 21, 22, 23, or 24 of the alleles described herein.

In certain embodiment, the method comprises detecting in the biological sample the presence of a panel of one or more single nucleotide polymorphisms (SNPs) selected from the group consisting of rs4315565, rs2154237, rs9828024, rs12105972, rs3943102 and rs2296651, or a complementary sequence thereof, or a complementary sequence thereof, wherein the presence of the panel of the one or more of the SNPs predicts sustained clinical response in the subject, and the absence of all of the SNPs predicts a relapse in the subject. In further embodiments, the one or more SNPs are associate with time to relapse with a p-value of less than 0.05, preferably less than 0.01. In further embodiments, the one or more SNPs are associate with time to relapse with a p-value of less than 0.05, preferably less than 0.01. The panel of SNPs can contain 1, 2, 3, 4, 5, or 6 of the SNPs described herein.

In further embodiment, the method comprises detecting in the biological sample the presence of a panel of one or more single nucleotide polymorphisms (SNPs) selected from the group consisting of rs2154237 and rs2296651, or a complementary sequence thereof, wherein the presence of the panel of the one or more of the SNPs predicts sustained clinical response in the subject, and the absence of all of the SNPs predicts a relapse in the subject. In further embodiments, the one or more SNPs are associate with time to relapse with a p-value of less than 0.05, preferably less than 0.01. The panel of SNPs can contain 1 or 2 of the SNPs described herein.

In certain embodiments, the treatment for CHB infection is a HBV DAA treatment.

In certain embodiments, the treatment for CHB infection is a NUC treatment.

In certain embodiments, the method further comprises detecting one or more additional biomarkers associated with the relapse. Examples of such biomarkers include, but are not limited to, the level of HBsAg at the end-of-treatment, the level of HBeAg prior to treatment, HBV DNA level, ALT, AST, HBV RNA.

Provided herein are also methods of treating a chronic hepatitis B (CHB) infection in a subject in need thereof using a panel of SNPs described herein.

In one general aspect, the application provides a method of treating a chronic hepatitis B (CHB) infection in a subject in need thereof, the method comprises:
a. administering to the subject a therapeutically effective amount of a HBV direct-acting antiviral agent (DAA) to treat the CHB infection;
b. discontinuing the HBV DAA treatment when the CHB infection is suppressed in the subject;
c. detecting in a biological sample obtained from the subject the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, s2934456, rs77586835, rs75876539, rs8050261, rs1542951, rs231770, rs9277535, rs3130542, rs7574865, rs2296651, and rs1419881, or a complementary sequence thereof; and
d. monitoring relapse in the subject two years or later after the discontinuation of the HBV DAA treatment, if the panel of the one or more SNPs is detected in the biological sample; or monitoring relapse in the subject prior to two years after the discontinuation of the HBV DAA treatment, if none of the one or more SNPs is detected in the biological sample.

In certain embodiments, the panel comprises at least two SNPs selected from the group consisting ofrs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, s2934456, rs77586835, rs75876539, rs8050261, rs1542951, rs231770, rs9277535, rs3130542, rs7574865, rs2296651, and rs1419881, or a complementary sequence thereof.

In certain embodiments, provided are method of predicting a relapse after discontinuation of a HBV DAA treatment in a subject diagnosed with CHB using a panel according to an embodiment of the invention. The method comprises: (a) obtaining a biological sample from a subject diagnosed with CHB; (b) determining whether the biological sample comprises one or more single nucleotide polymorphisms (SNPs) selected from the panel of SNPs.

Preferably, the method further comprising administering to the subject a treatment, wherein the treatment is a HBV DAA treatment if the panel of the one or more SNPs is detected in the biological sample, or the treatment is a non-DAA treatment if none of the SNPs is detected in the biological sample.

In certain embodiments, the HBV DAA treatment for the CHB infection is a NUC treatment.

Provided herein are also methods of treating a chronic hepatitis B (CHB) infection in a subject in need thereof, the method comprising:
a. detecting in a biological sample obtained from the subject the presence of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof; and
b. administering to the subject a therapeutically effective amount of a HBV direct-acting antiviral agent (DAA) if the panel of the one or more SNPs is detected in the biological sample, or administering to the subject a therapeutically effective amount of a non-DAA agent if none of the SNPs is detected in the biological sample.

In certain embodiments, the HBV DAA treatment for the CHB infection is a NUC treatment.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, or a complementary sequence thereof.

In certain embodiments, the one or more single nucleotide polymorphisms (SNPs) is selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof.

In certain embodiments, the one or more single nucleotide polymorphisms (SNPs) is selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof.

In certain embodiments, the one or more single nucleotide polymorphisms (SNPs) is selected from the group consisting of rs4668818, rs948006, rs2934456, rs77586835, rs75876539, rs8050261, rs1542951, rs231770, and rs9277535, or a complementary sequence thereof.

In certain embodiments, the one or more single nucleotide polymorphisms (SNPs) is selected from the group consisting of rs4668818, rs948006, rs2934456, rs77586835, rs75876539, rs8050261, and rs1542951, or a complementary sequence thereof.

In certain embodiments, the one or more single nucleotide polymorphisms (SNPs) is selected from the group consisting of rs4668818, rs948006, rs2934456, rs75876539, rs8050261, and rs1542951, or a complementary sequence thereof.

In certain embodiments, the method further comprises detecting in the biological sample the presence of a panel of one or more alleles selected from the group consisting of allele T in rs4668818, allele C in rs948006, allele A in rs2934456, allele C in rs75876539, allele G in rs8050261, and allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, and allele A in rs1419881, or a complementary sequence thereof.

In certain embodiments, the method further comprises detecting in the biological sample the presence of a panel of one or more alleles selected from the group consisting of allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, and allele G in rs1419881, or a complementary sequence thereof.

In certain embodiments, the method further comprises detecting in the biological sample the presence of a panel of one or more alleles selected from the group consisting of allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, and allele C in rs10235518, or a complementary sequence thereof.

In certain embodiments, the method further comprises detecting in the biological sample the presence of a panel of one or more alleles selected the group consisting of allele T in rs4668818, allele C in rs948006, allele A in rs2934456, allele T in rs77586835, allele C in rs75876539, allele G in rs8050261, and allele A in rs1542951, or a complementary sequence thereof.

In certain embodiment, the one or more single nucleotide polymorphisms (SNPs) is selected from the group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof.

In certain embodiment, the one or more single nucleotide polymorphisms (SNPs) is selected from the group consisting of rs4315565, rs2154237, rs9828024, rs12105972, rs3943102 and rs2296651, or a complementary sequence thereof.

In further embodiment, the one or more single nucleotide polymorphisms (SNPs) is selected from the group consisting of rs2154237 and rs2296651, or a complementary sequence thereof.

In certain embodiments, the method further comprises detecting in the biological sample the presence of a panel of one or more alleles selected from the group consisting of allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, and allele T in rs7459445, or a complementary sequence thereof.

In certain embodiments, the method further comprises detecting in the biological sample the presence of a panel of one or more alleles selected from the group consisting of allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, and allele C in rs7459445, or a complementary sequence thereof.

In certain embodiments, the presence of absence of the one or more SNPs or alleles in the biological sample is determined using any method known to one skilled in the art.

In certain embodiments, the subject is treated with a HBV DAA treatment such as a NUC treatment, if the panel of the one or more SNPs is detected in the biological sample. The NUC can be tenofovir, entecavir, lamivudine, adefovir, or telbivudine.

In certain embodiments, the subject is treated with a non-DAA treatment, if there is no one or more SNPs determined in the biological sample.

In certain embodiments, the subject achieves HBV DNA < 60 IU/mL, ALT < 80 U/L, or HBeAg negative at or after one year, two years, three years, or four years, or anytime in between, after the HBV DAA treatment. In further embodiments, the subject then discontinues the HBV DAA treatment.

In further embodiments, the subject has no virological relapse or clinical relapse at or after 3 months, 6 months, 12 months, 18 months, 24 months, or 36 months after the discontinuation of the HBV DAA treatment, or anytime in between, and wherein the virological relapse is identified as HBV DNA ≥ 2000 IU/ml or HBeAg positive, and the clinical relapse is identified as i) HBV DNA ≥ 2000 IU/ml or HBeAg positive, and ii) ALT ≥ 80 U/L.

Provided herein are also methods of treating a chronic hepatitis B (CHB) infection in a subject in need thereof, the method comprising:
a. administering to the subject a therapeutically effective amount of a HBV direct-acting antiviral agent (DAA);
b. detecting in a biological sample obtained from the subject the presence of a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881, or a complementary sequence thereof; and
c. continuing treating the subject with the HBV DAA if the panel of the one or more SNPs is detected in the biological sample, or switching from the HBV DAA treatment to a non-DAA treatment if none of the SNPs is detected in the biological sample.

In certain embodiments, the HBV DAA treatment for the CHB infection is a NUC treatment.

In certain embodiments, the NUC treatment can be tenofovir, entecavir, lamivudine, adefovir, or telbivudine.

According to embodiments of the invention, the biological sample is obtained from the subject before, or after the subject is treated a treatment. Preferably, the presence or absence of the one or more SNPs in the biological sample is determined using any method known to one skilled in the art.

In certain embodiments, the determination of the one of more SNPs is performed before the HBV DAA treatment is administered to the subject.

In certain embodiments, the determination of the one of more SNPs is performed after the HBV DAA treatment is administered to the subject.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, or a complementary sequence thereof.

In certain embodiments, the one or more single nucleotide polymorphisms (SNPs) is selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof.

In certain embodiments, the one or more single nucleotide polymorphisms (SNPs) is selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof.

In certain embodiments, the one or more single nucleotide polymorphisms (SNPs) is selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof.

In certain embodiments, the one or more single nucleotide polymorphisms (SNPs) is selected from the group consisting of rs4668818, rs948006, rs2934456, rs77586835, rs75876539, rs8050261, rs1542951, rs231770, and rs9277535, or a complementary sequence thereof.

In certain embodiments, the one or more single nucleotide polymorphisms (SNPs) is selected from the group consisting of rs4668818, rs948006, rs2934456, rs77586835, rs75876539, rs8050261, rs1542951, or a complementary sequence thereof.

In certain embodiments, the one or more single nucleotide polymorphisms (SNPs) is selected from the group consisting of rs4668818, rs948006, rs2934456, rs75876539, rs8050261, and rs1542951, or a complementary sequence thereof.

In certain embodiments, the method further comprises detecting in the biological sample the presence of a panel of one or more alleles selected from the group consisting of allele T in rs4668818, allele C in rs948006, allele A in rs2934456, allele C in rs75876539, allele G in rs8050261, and allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, and allele A in rs1419881, or a complementary sequence thereof.

In certain embodiments, the method further comprises detecting in the biological sample the presence of a panel of one or more alleles selected from the group consisting of allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, and allele G in rs1419881, or a complementary sequence thereof.

In certain embodiments, the method further comprises detecting in the biological sample the presence of a panel of one or more alleles selected from the group consisting of allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, and allele C in rs10235518, or a complementary sequence thereof.

In certain embodiments, the method further comprises detecting in the biological sample the presence of a panel of one or more alleles selected the group consisting of allele T in rs4668818, allele C in rs948006, allele A in rs2934456, allele T in rs77586835, allele C in rs75876539, allele G in rs8050261, and allele A in rs1542951, or a complementary sequence thereof.

In certain embodiment, the one or more single nucleotide polymorphisms (SNPs) is selected from the group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof.

In certain embodiment, the one or more single nucleotide polymorphisms (SNPs) is selected from the group consisting of rs4315565, rs2154237, rs9828024, rs12105972, rs2154237 and rs2296651, or a complementary sequence thereof.

In further embodiment, the one or more single nucleotide polymorphisms (SNPs) is selected from the group consisting of rs2154237 and rs2296651, or a complementary sequence thereof.

In certain embodiments, the method further comprises detecting in the biological sample the presence of a panel of one or more alleles selected from the group consisting of allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, and allele T in rs7459445, or a complementary sequence thereof.

In certain embodiments, the method further comprises detecting in the biological sample the presence of a panel of one or more alleles selected from the group consisting of allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, and allele C in rs7459445, or a complementary sequence thereof.

In certain embodiments, the subject continues the HBV DAA treatment, if the panel of the one or more SNPs is detected in the biological sample.

In certain embodiments, the subject switches the prior HBV DAA treatment, if there is no one or more SNPs determined in the biological sample.

In certain embodiments, the subject achieves HBV DNA < 60 IU/mL, ALT < 80 U/L, or HBeAg negative at or after one year, two years, three years, or four years, or anytime in between, after the HBV DAA treatment. In further embodiments, the subject then discontinues the HBV DAA treatment.

In further embodiments, the subject has no virological relapse or clinical relapse at or after 3 months, 6 months, 12 months, 18 months, 24 months, or 36 months after the discontinuation of the HBV DAA treatment, or anytime in between, and wherein the virological relapse is identified as HBV DNA ≥ 2000 IU/ml or HBeAg positive, and the clinical relapse is identified as i) HBV DNA ≥ 2000 IU/ml or HBeAg positive, and ii) ALT ≥ 80 U/L.

In certain embodiments, the sample is a tissue sample, a cellular sample, or a blood sample. Preferably, the sample is a blood sample.

In certain embodiments, the HBV DAA treatment for the CHB infection is a NUC treatment. The NUC can be any nucleotide or nucleoside analogue effective against CHB. Preferably, the NUC is selected from the group consisting of tenofovir, entecavir, lamivudine, adefovir, and telbivudine.

In certain embodiments, the CHB infection is suppressed and the treatment can be discontinued. In one embodiment, the subject discontinues a HBV DAA treatment when the subject achieves HBV DNA < 60 IU/mL, ALT < 80 U/L, or HBeAg negative at or after one year, two years, three years, or four years, or anytime in between, after the HBV DAA treatment. Preferably, the subject achieves HBsAg < 100 IU/mL at the discontinuation of the HBV DAA treatment.

In certain embodiment, the method further comprises measuring HBV DNA, ALT, and HBsAg, at or after 3 months, 6 months, 12 months, 18 months, 24 months, or 36 months after the discontinuation of the HBV DAA treatment, or anytime in between.

In certain embodiment, the subject has no virological relapse at or after 3 months, 6 months, 12 months, 18 months, 24 months, or 36 months after the discontinuation of the HBV DAA treatment, or anytime in between, and the virological relapse is identified as HBV DNA ≥ 2000 IU/ml or HBeAg positive.

In certain embodiment, the subject has no clinical relapse at or after 3 months, 6 months, 12 months, 18 months, 24 months, or 36 months after the discontinuation of the HBV DAA treatment, or anytime in between, and the clinical relapse is identified as i) HBV DNA ≥ 2000 IU/ml or HBeAg positive, and ii) ALT ≥ 80 U/L.

In another general aspect, the application relates to a nucleotide or nucleoside analogue (NUC) for use in a treatment of a chronic hepatitis B (CHB) infection in a subject in need thereof, which comprises:
a. detecting in a biological sample obtained from the subject a panel of one or more single nucleotide polymorphisms (SNPs) selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, s2934456, rs77586835, rs75876539, rs8050261, rs1542951, rs231770, rs9277535, rs3130542, rs7574865, rs2296651, and rs1419881, or a complementary sequence thereof, and
b. administering to the subject a therapeutically effective amount of the NUC if the panel of the one or more SNPs is detected in the biological sample; or administering to the subject a therapeutically effective amount of a non-NUC agent if none of the SNPs is detected in the biological sample.

In certain embodiment, the one or more SNPs are associated with time to relapse.

In certain embodiment, the one or more SNPs are associated with the time to relapse with a p-value of less than 0.05, preferably less than 1.0E-05, more preferably 5.40E-06 or less.

In certain embodiment, the one or more SNPs are associated with the time to relapse with a p-value of less than 0.05, and the one or more SNPs are selected from the group consisting of rs231770, rs9277535, rs3130542, rs7574865, rs2296651, and rs1419881, or a complementary sequence thereof.

In certain embodiment, the one or more SNPs are associated with the time to relapse with a p-value of 0.001 to 0.05, and the one or more SNPs are selected from the group consisting of rs231770, rs9277535, rs7574865, rs2296651, and rs1419881, or a complementary sequence thereof.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, and rs1542951, or a complementary sequence thereof. In further embodiments, the one or more SNPS are associated with time to relapse with a p-value of less than 0.05, preferably less than 1.0E-05, more preferably 5.40E-06 or less.

In certain embodiment, the one or more SNPs are selected from the group consisting of rs4668818, rs948006, rs2934456, rs75876539, rs8050261, rs1542951, rs7534054, rs12105972, rs7629161, rs9828024, rs7670984, rs2163787, rs924446, rs12199613, rs1053403, rs2767035, rs78045374, rs117634357, rs2236895, rs7646021, rs17152247, rs10235518, and rs1419881, or a complementary sequence thereof. In further embodiments, the one or more SNPs are associate with time to relapse with a p-value of less than 0.05, preferably less than 1.0E-05, more preferably 5.40E-06 or less.

In certain embodiments, the NUC for use further comprises detecting in the biological sample the presence of a panel of one or more alleles selected from the group consisting of allele T in rs4668818, allele C in rs948006, allele A in rs2934456, allele C in rs75876539, allele G in rs8050261, and allele A in rs1542951, allele A in rs7534054, allele G in rs12105972, allele T in rs7629161, allele G in rs9828024, allele C in rs7670984, allele G in rs2163787, allele C in rs924446, allele C in rs12199613, allele G in rs1053403, allele C in rs2767035, allele T in rs78045374, allele C in rs117634357, allele G in rs2236895, allele T in rs7646021, allele C in rs17152247, allele C in rs10235518, and allele A in rs1419881, or a complementary sequence thereof.

In certain embodiments, the NUC for use further comprises detecting in the biological sample the presence of a panel of one or more alleles selected from the group consisting of allele CC in rs4668818, allele TT in rs948006, allele GG in rs2934456, allele A in rs75876539, allele C in rs8050261, and allele C in rs1542951, allele C in rs7534054, allele C in rs12105972, allele C in rs7629161, allele A in rs9828024, allele A in rs7670984, allele A in rs2163787, allele T in rs924446, allele T in rs12199613, allele A in rs1053403, allele T in rs2767035, allele C in rs78045374, allele TT in rs117634357, allele TT in rs2236895, allele GG in rs7646021, allele TT in rs17152247, allele TT in rs10235518, and allele G in rs1419881, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs3130542, rs7574865, rs2296651 and rs1419881, or a complementary sequence thereof. In further embodiments, the one or more SNPs are associate with time to relapse with a p-value of less than 0.05, preferably less than 1.0E-05, more preferably 5.40E-06 or less.

In certain embodiment, the one or more SNPs are associated with time to relapse with a p-value of less than 0.05, preferably less than 1.0E-05, more preferably 5.40E-06 or less, and the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518 and rs3130542, or a complementary sequence thereof.

In certain embodiment, the one or more SNPs are selected from the group consisting of rs7534054, rs4315565, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, and rs10235518, or a complementary sequence thereof. In further embodiments, the one or more SNPs are associate with time to relapse with a p-value of less than 0.05, preferably less than 1.0E-05, more preferably 5.40E-06 or less.

In certain embodiments, the NUC for use further comprises detecting in the biological sample the presence of a panel of one or more alleles selected from the group consisting of allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, and allele C in rs10235518, or a complementary sequence thereof.

In certain embodiment, the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, rs10235518, and rs1419881, or a complementary sequence thereof. In further embodiments, the one or more SNPs are associate with time to relapse with a p-value of less than 0.05.

In further embodiments, the one or more SNPs are associate with time to relapse with a p-value of less than 0.05, preferably less than 1.0E-05, more preferably 5.40E-06 or less, and the one or more SNPs are selected from the group consisting of rs7534054, rs12105972, rs7629161, rs9828024, rs7670984, rs12645094, rs2163787, rs924446, rs1053403, rs2767035, rs3943102, rs117634357, rs2236895, rs7646021, rs17152247, and rs10235518, or a complementary sequence thereof.

In certain embodiment, the one or SNPs are selected from the group consisting of rs4668818, rs948006, rs2934456, rs77586835, rs75876539, rs8050261, rs1542951, rs231770, and rs9277535, or a complementary sequence thereof. In further embodiments, the one or more SNPs are associate with time to relapse with a p-value of less than 0.05, preferably less than 1.0E-05, more preferably 5.40E-06 or less.

In certain embodiment, the one or more SNPs are selected from the group consisting of rs4668818, rs948006, rs2934456, rs77586835, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less.

In certain embodiments, the NUC for use further comprises detecting in the biological sample the presence of a panel of one or more alleles selected from the group consisting of allele T in rs4668818, allele C in rs948006, allele A in rs2934456, allele T in rs77586835, allele C in rs75876539, allele G in rs8050261, and allele A in rs1542951, or a complementary sequence thereof.

In certain embodiment, the one or more SNPs are selected from the group consisting of rs4668818, rs948006, rs2934456, rs75876539, rs8050261, and rs1542951, or a complementary sequence thereof. In further embodiments, the one or more SNPs are associate with time to relapse with a p-value of less than 0.05, preferably less than 1.0E-05, more preferably 5.40E-06 or less.

In one embodiment, the SNP is rs2296651 or a complementary sequence thereof.

In one embodiment, the SNP is rs231770 or a complementary sequence thereof.

In certain embodiment, the one or more SNPs are selected from group consisting of rs7534054, rs180001, rs4315565, rs2154237, rs10235518, rs9828024, rs924446, rs12105972, rs2767035, rs7205040, rs3943102, rs12645094, rs73371840, rs7629161, rs1053403, rs552219, rs2296651, rs3130542, rs2394952, rs11896590, rs17152258, rs1994245, rs12199613, and rs7459445, or a complementary sequence thereof. In further embodiments, the one or more SNPs are associate with time to relapse with a p-value of less than 0.05.

In certain embodiments, the NUC for use further comprises detecting in the biological sample the presence of a panel of one or more alleles selected from the group consisting of allele A in rs7534054, allele G in rs180001, allele A in rs4315565, allele G in rs2154237, allele CC in rs10235518, allele G in rs9828024, allele C in rs924446, allele G in rs12105972, allele C in rs2767035, allele T in rs7205040, allele C in rs3943102, allele A in rs12645094, allele C in rs73371840, allele T in rs7629161, allele G in rs1053403, allele A in rs552219, allele A in rs2296651, allele G in rs3130542, allele A in rs2394952, allele G in rs11896590, allele C in rs17152258, allele T in rs1994245, allele C in rs12199613, and allele T in rs7459445, or a complementary sequence thereof.

In certain embodiments, the NUC for use further comprises detecting in the biological sample the presence of a panel of one or more alleles selected from the group consisting of allele C in rs7534054, allele A in rs180001, allele G in rs4315565, allele T in rs2154237, allele T in rs10235518, allele A in rs9828024, allele T in rs924446, allele C in rs12105972, allele T in rs2767035, allele C in rs7205040, allele T in rs3943102, allele C in rs12645094, allele T in rs73371840, allele C in rs7629161, allele A in rs1053403, allele G in rs552219, allele G in rs2296651, allele A in rs3130542, allele G in rs2394952, allele A in rs11896590, allele T in rs17152258, allele C in rs1994245, allele T in rs12199613, and allele C in rs7459445, or a complementary sequence thereof.

In certain embodiment, the one or more SNPs are selected from the group consisting of rs4315565, rs2154237, rs9828024, rs12105972, rs3943102 and rs2296651, or a complementary sequence thereof, or a complementary sequence thereof. In further embodiments, the one or more SNPs are associate with time to relapse with a p-value of less than 0.05, preferably less than 0.01. In further embodiments, the one or more SNPs are associate with time to relapse with a p-value of less than 0.05, preferably less than 0.01.

In further embodiment, the method comprises detecting in the biological sample the presence of a panel of one or more single nucleotide polymorphisms (SNPs) selected from the group consisting of rs2154237 and rs2296651, or a complementary sequence thereof. In further embodiments, the one or more SNPs are associate with time to relapse with a p-value of less than 0.05, preferably less than 0.01.

In certain embodiments, the sample is a tissue sample, a cellular sample, or a blood sample. Preferably, the sample is a blood sample.

In certain embodiments, the NUC is selected from the group consisting of tenofovir, entecavir, lamivudine, adefovir, and telbivudine.

In certain embodiments, the non-NUC agent is interferon.

In certain embodiments, the subject achieves HBV DNA < 60 IU/mL, ALT < 80 U/L, or HBeAg negative at or after one year, two years, three years, or four years, or anytime in between, after the NUC treatment. In further embodiments, the subject then discontinues the NUC treatment.

In further embodiments, the subject has no virological relapse or clinical relapse at or after 3 months, 6 months, 12 months, 18 months, 24 months, or 36 months after the discontinuation of the NUC treatment, or anytime in between, and wherein the virological relapse is identified as HBV DNA ≥ 2000 IU/ml or HBeAg positive, and the clinical relapse is identified as i) HBV DNA ≥ 2000 IU/ml or HBeAg positive, and ii) ALT ≥ 80 U/L.

In certain embodiments, the CHB infection is suppressed and the treatment can be discontinued. In one embodiment, the subject discontinues the NUC treatment when the subject achieves HBV DNA < 60 IU/mL, ALT < 80 U/L, or HBeAg negative at or after one year, two years, three years, or four years, or anytime in between, after the NUC treatment. Preferably, the subject achieves HBsAg < 100 IU/mL at the discontinuation of the NUC treatment.

In certain embodiment, the method further comprises measuring HBV DNA, ALT, and HBsAg, at or after 3 months, 6 months, 12 months, 18 months, 24 months, or 36 months after the discontinuation of the NUC treatment, or anytime in between.

In certain embodiment, the subject has no virological relapse at or after 3 months, 6 months, 12 months, 18 months, 24 months, or 36 months after the discontinuation of the NUC treatment, or anytime in between, and the virological relapse is identified as HBV DNA ≥ 2000 IU/ml or HBeAg positive.

In certain embodiment, the subject has no clinical relapse at or after 3 months, 6 months, 12 months, 18 months, 24 months, or 36 months after the discontinuation of the NUC treatment, or anytime in between, and the clinical relapse is identified as i) HBV DNA ≥ 2000 IU/ml or HBeAg positive, and ii) ALT ≥ 80 U/L.

Any method known in the art or described herein can be used to detect the presence of a SNP in view of the present disclosure. According to embodiments of the application, the SNP or allele is determined by a method selected from the group consisting of DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

### EXAMPLES

### Example 1: Identification and Evaluation of Virological and Host Genetic Markers Associated with Time to Clinical Relapse

The objective of the current study was to evaluate virological and host genetic markers that can be associated with relapse and sustained response in chronic hepatitis B chronic hepatitis B (CHB) patients following discontinuation of direct antiviral treatment.

### Materials and Methods

Patients and Study Design: In this multi-center prospective study in Taiwan, 242 CHB patients were enrolled while in their last half year of a minimum 3-year treatment regimen with direct antivirals. Of them, 47 were excluded due to protocol violation, screening failure, lost to follow-up, withdrawal by the subject, use of immune related therapy, or on-treatment clinical relapse. An additional 14 were excluded as the analysis required patients to be hepatitis B e-antigen (HBeAg) negative, hepatitis virus (HBV) DNA < 60 IU/mL, and alanine transaminase (ALT) < 80 U/L at the last on-treatment visit, leaving 186 patients eligible for analysis.

Patients were followed for up to two years after treatment discontinuation while assessing virological and clinical relapse, provided that treatment was not re-initiated. Virological relapse was defined as HBV DNA ≥ 2000 IU/mL. Clinical relapse was defined as ALT level ≥ 2x ULN in addition to a virological relapse. A patient was denoted a sustained clinical responder in case no clinical and no virological relapse occurred during the entire follow-up period after treatment cessation.

In addition, biochemical relapse was defined as ALT level ≥ 2x ULN, and transient (virological/clinical) relapse was defined as virological/clinical relapse after stop of treatment and HBV DNA < 2000 IU/mL and ALT < 80 U/L at last observed post-treatment observation.

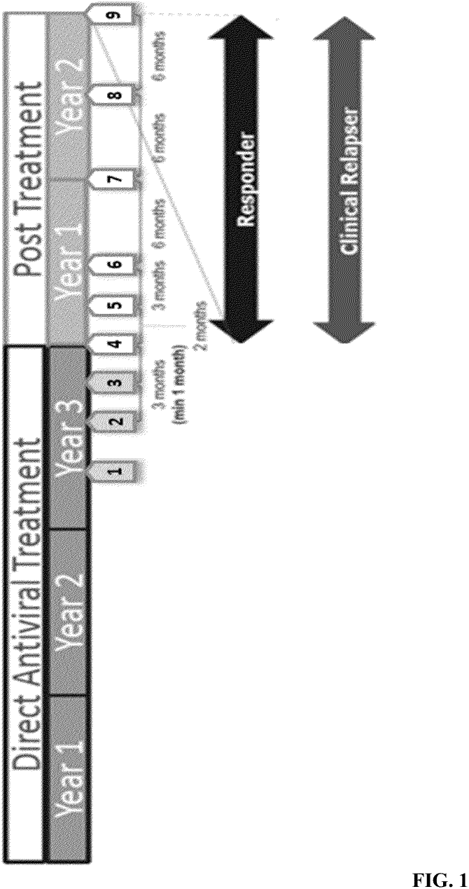

A diagram of the study design is provided in FIG. 1.

Serology: During the on-treatment period, blood samples were collected at 3 time points, with the last one collected around the last day of treatment. In the follow-up period after treatment discontinuation additional blood samples were collected at 3 or 6 months intervals, provided that treatment was not re-initiated.

Genetic Data: The DNA of each subject was hybridized to two different genotyping arrays: Axiom UK Biobank Chip and Axiom Asia PRMA from Thermo Fisher Scientific (Waltham, MA). The genotype calling was performed on biallelic SNPs using Affymetrix power tools (2.8.6) following the manufacturer’s instructions. The genotype calling was performed independently for analysis batch as suggested by the manufacturer.

The resulting genotype files were converted to plink BED files. For each subject the genotypes from the Axiom UK Biobank and the Asia PRMA Chip were merged using PLINK (v1.9). The Asia PRMA Chip was used as a reference, and complemented with the probes from the Axiom UK Biobank chip that were not in the Asia PRMA Chip. The merging of the genotypes resulted in a dataset composed of 1,295,727 SNPs and 183 subjects.

Prior to the statistical analysis the 1,295,727 SNPs were filtered based on the following quality control criteria: minor allele frequency > 10% and genotype missing rate < 5% of the subjects. Heterochromosomal and mitochondrial SNPs were excluded. A total of 383,634 SNPs passed the quality control criteria and were used for the statistical analyses.

Statistical Analysis: Continuous variables are presented as means ± standard deviations, categorical variables by counts and percentages. The Student t test and Fisher exact test were used to compare continuous and categorical covariates, respectively, between groups. Cumulative incidence of virological and clinical relapse after stop of treatment was assessed by Kaplan-Meier curves including the log-rank test. Cox proportional hazard regression analysis was applied for univariate and multivariate models of time to virological and clinical relapse. To assess associations to sustained clinical response, logistic regression analysis was performed. All statistical analyses were executed within the R(3.4.3) statistical software. Statistical tests were two-sided and considered significant at the 0.05 significance level.

Statistical Analysis of Genetics Data: All the statistical analyses were performed in R (3.4.3). The association between the SNPs and the time to clinical relapse and time to viral relapse was assessed using a Cox proportional hazard model, considering the prior treatment.
Surv(TIME_VIRAL_RELAPSE,STATUS_VIRAL_RELAPSE) ~ GENETIC + LAST_TREATMENT
Surv(TIME_CLINICAL _RELAPSE,STATUS_CLINICAL_RELAPSE) ~ GENETIC + LAST_TREATMENT

Where the GENETIC term corresponds to the SNP encoded using the additive (AA=0, AB=1, BB=2), dominant (AA=0, AB=1, BB=1) or recessive (AA=0, AB=0, BB=1) genetic models. In each of these models B allele was set as the minor allele in the analysis population.

The time to viral and clinical relapse models were run independently for every SNP and each of the three genetic models. For the dominant and recessive genetic models, the analysis was run only if the resulting minor genotype frequencies of the SNP represented >= 10% of the population (18 subjects). This step was implemented in order to avoid detecting non-robust associations due to low genotype counts.

Those models were applied, on one hand SNPs on 22 candidate genes (already reported as genetically associated with chronic HBV infection; see Table 1) and on the other hand on the full genotyping array.

TABLE 1

Candidate Genes

| | | | |
|---|---|---|---|
| SNPSNP | RSID | GENE | COMMENT |
| AX-112063628 | rs12979860 | IFNL4 | IL28B |
| AX-40445375 | rs12979860 | IFNL4 | IL28B |
| AX-11432694 | rs3077 | HLA-DPA1 | HLA DPB1 |
| AX-11665728 | rs8099917 | ---- | IL28B |
| AX-11677618 | rs9277535 | HLA-DPB1 | HLA DPB1 |
| AX-12524927 | rs2296651 | SLC10A1 | NTCP |
| AX-11382422 | rs2291617 | METTL21B | METTL21B in Strong LD (South Chinese pop) with rs4646536 and rs10877012 |
| AX-11384541 | rs231770 | CTLA4 | CTLA4 in Strong LD (South Chinese pop) with rs231775 |
| AX-11630908 | rs7574865 | STAT4 | STAT4 |
| AX-11419599 | rs2856718 | HLA-DQB1 | HLA-DQB1 |
| AX-11625220 | rs7453920 | HLA-DQB2 | HLA-DQB2 |
| AX-41954239 | rs652888 | EHMT2 | EHMT2 |
| AX-165873764 | rs9272105 | HLA-DQA1 | HLA-DQA1 |
| AX-41957017 | rs9272105 | HLA-DQA1 | HLA-DQA1 |
| AX-11435438 | rs3130542 | HLA-C | HLA-C |
| AX-41950487 | rs1419881 | TCF19 | TCF19 |
| AX-11202033 | rs12356193 | SLC16A9 | SLC16A9 |
| AX-11511204 | rs455804 | GRIK1 | GRIK1 |
| AX-12582596 | rs4821116 | UBE2L3 | UBE2L3 |
| AX-15654248 | rs9692372 | ---- | replaces rs6462008 (LD-r2 of 0.78 in Han Chinese population) |
| AX-15121359 | rs169083 | ---- | replaces rs171941 (LD-r2 of 0.79 in Han Chinese population) |
| AX-16645527 | rs2000478 | ----- | replaces rs7944135 (LD-r2 of 0.83 in Han Chinese population) |

The p-value of the genetic term was corrected for multiple testing using the Benjamini-Hochberg procedure (Benjamini, Y. and Hochberg, Y. “Controlling the false discovery rate: a practical and powerful approach to multiple testing,” J. R. Statist, Soc. B, 1995, 57:289-300) for false discovery rate (FDR) analysis independently for both approaches. The multiple testing correction was performed independently for each genetic model (additive, dominant or recessive)

Selection of Genetic Hits: A graphical approach was applied to select SNP hits (plotting FDR in function of the rank of raw p-values). Based on this approach, SNPs with FDR<33.6% were considered as candidate hits for time to clinical relapse and SNPs with an FDR <= 5% were selected as statistical hits for time to viral relapse. The final list of hits was composed of the union of SNPs passing the FDR selection in either the additive, dominant or recessive genetic models. In cases where the SNP was identified as a hit in more than one genetic model the precedence was given to the additive model, followed by the dominant model.

In whole genome scans it is common to identify associations with SNPs that belong to the same linkage-disequilibrium block and that therefore convey the same statistical information. To take this into account the list of statistical hits was clumped using PLINK (v1.9). This procedure selects the SNP with the lowest p-value, representative of a linkage-disequilibrium block (r2 = 0.8).

### Results

Study Population: Table 2 and Table 3 provide an overview of the clinical characteristics of the study population. In total, 101 patients completed the study, for 83 patients nucleos(t)ide analog (NA) therapy was re-initiated due to relapse. One patient died, and one patient was diagnosed with hepatocellular carcinoma during the study period. The follow-up time after stop of treatment ranges from 38 to 814 days, with a mean and median period of follow-up of 482 and 637 days, respectively.

TABLE 2

| Clinical charachteristics of study population (N = 186) | |
|---|---|
| Age (years) | 50.8 ± 11.3 |
| Gender | |
| Male | 142 (76.3%) |
| Female | 44 (23.7%) |
| Previous NA therapy | |
| Yes | 64 (34.4%) |
| No | 122 (65.6%) |
| Number of treatment free periods > 1 month | |
| 0 | 123 (66.1%) |
| 1 | 37 (19.9%) |
| 2 | 15 (8.1%) |
| 3 | 5 (2.7%) |
| 4 | 3 (1.6%) |
| 6 | 1 (0.5%) |
| Baseline HBeAg status | |
| Negativity prior to start of NA therapy | 142 (76.3%) |
| Negativity obtained during NA therapy | 44 (23.7%) |
| Treatment regimen | |
| Entecavir | 106 (57.0%) |
| Tenofovir | 65 (34.9%) |
| Other | 15 (8.1%) |
| Length NA therapy (years, mean ± SD) | 4.3 ± 1.9 |

TABLE 3

| Clinical charachteristics of study population (N = 186) | |
|---|---|
| On treatment viral suppression | 26 (14.0%) |
| No (HBV DNA all detected) | 38 (20.4%) |
| Consistent (HBV DNA all not detected) | 122 (65.6%) |
| Inconsistent (HBV DNA both detected and not detected) | |
| End of treatment HBV DNA | 96 (51.6%) |
| Detected | 90 (48.4%) |
| Not detected | |
| End of treatment HBsAg | 39 (21.0%) |
| < 100 IU/mL | 147 (79.0%) |
| ≥ 100 IU/mL | 5.6 ± 2.3 |
| End of treatment HBsAg (log IU/mL) | 0.16 ± 0.73 |
| End of treatment HBsAb (log mIU/mL) | 3.1 ± 0.47 |
| End of treatment ALT (log U/L) | 3.1 ± 0.29 |
| End of treatment AST (log U/L) | 5.5 ± 5.0 |
| 1 month follow-up HBV DNA (log IU/mL) | 5.5 ± 2.6 |
| 1 month follow-up HBsAg (log IU/mL) | 3.3 ± 0.79 |
| 1 month follow-up ALT (log U/L) | |

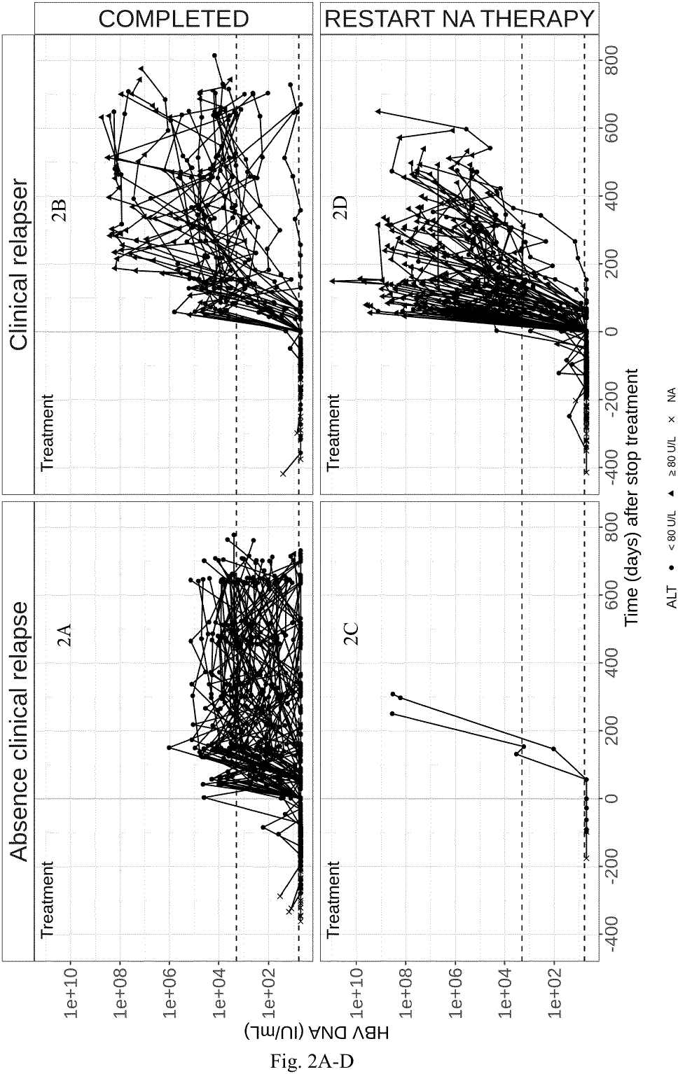

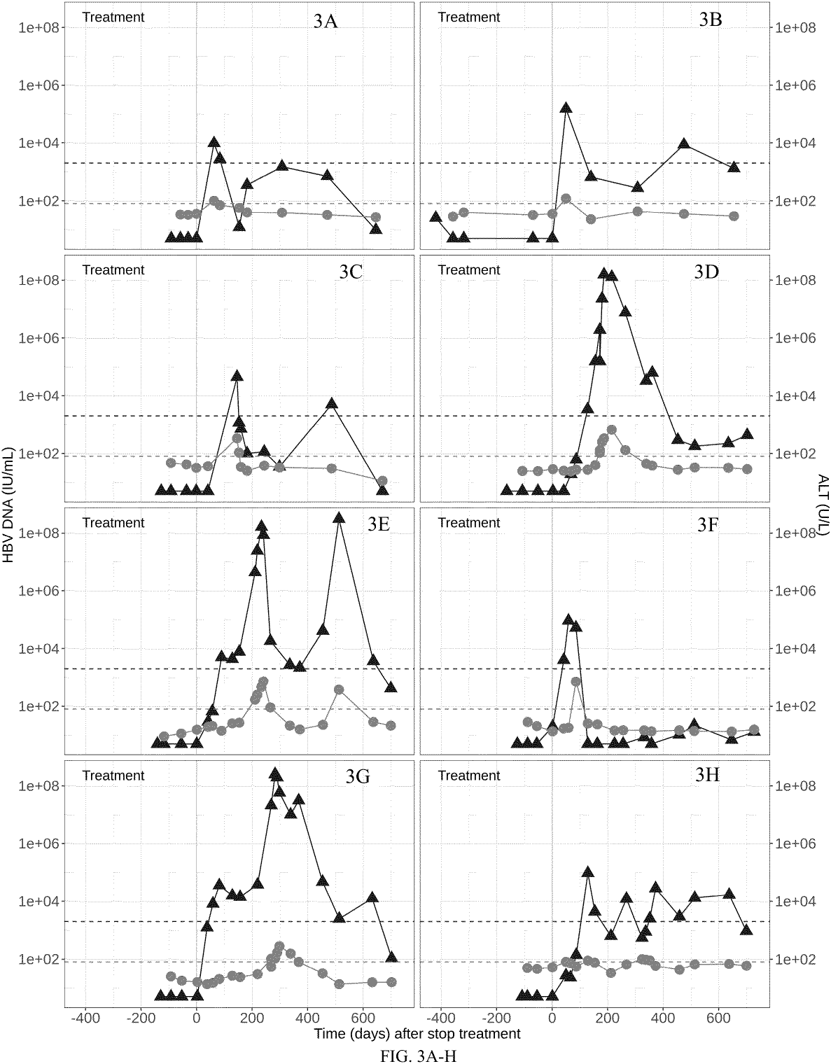

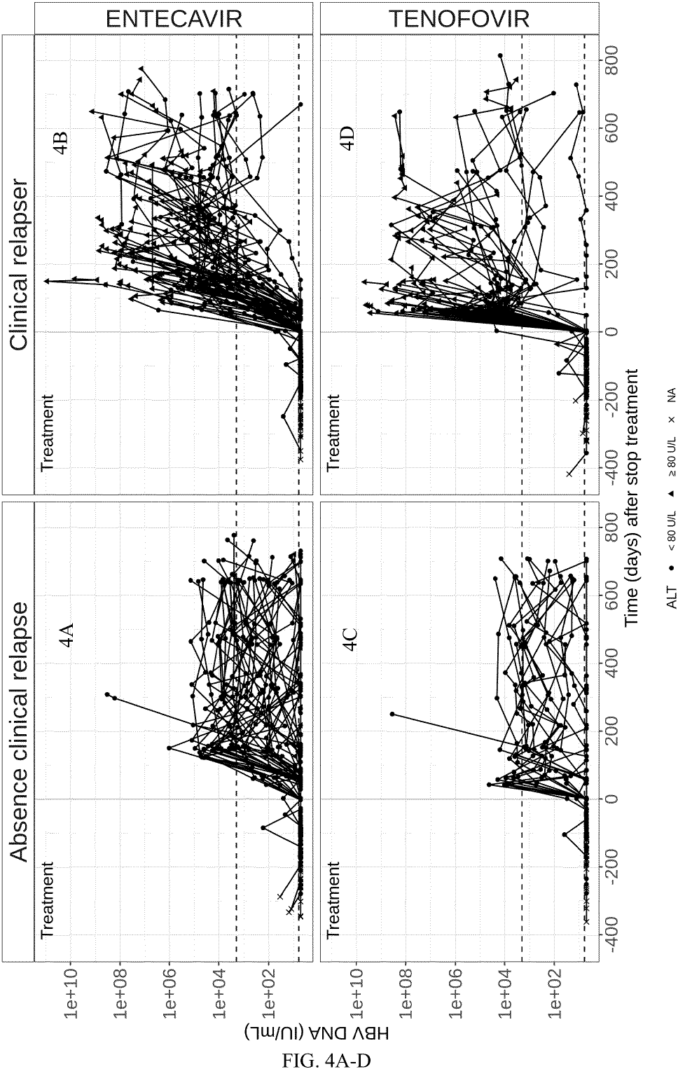

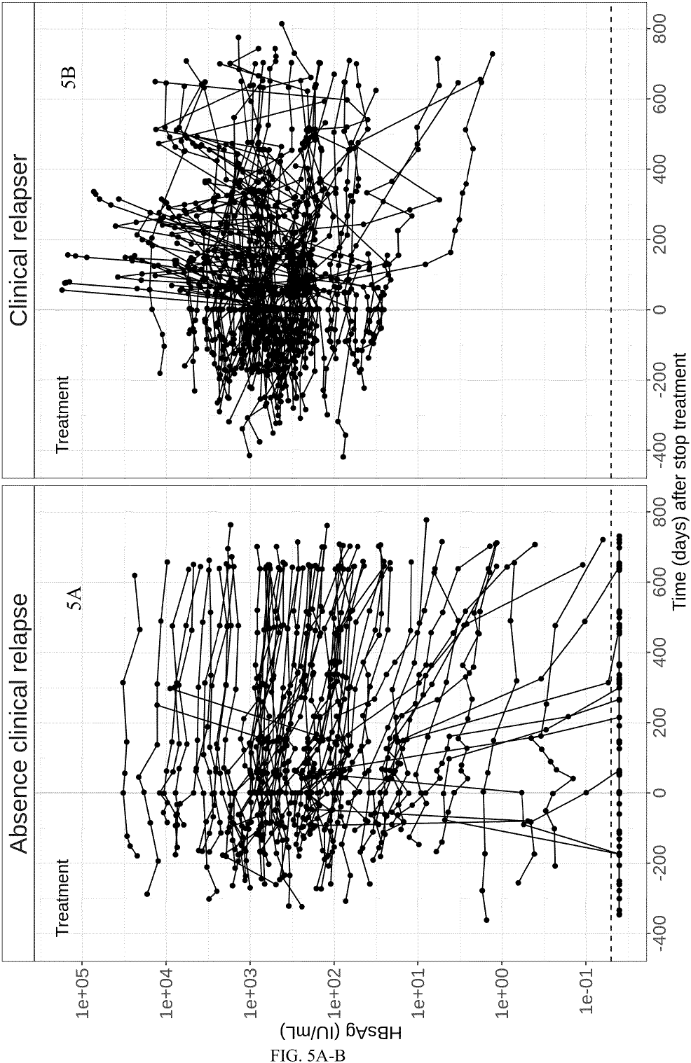

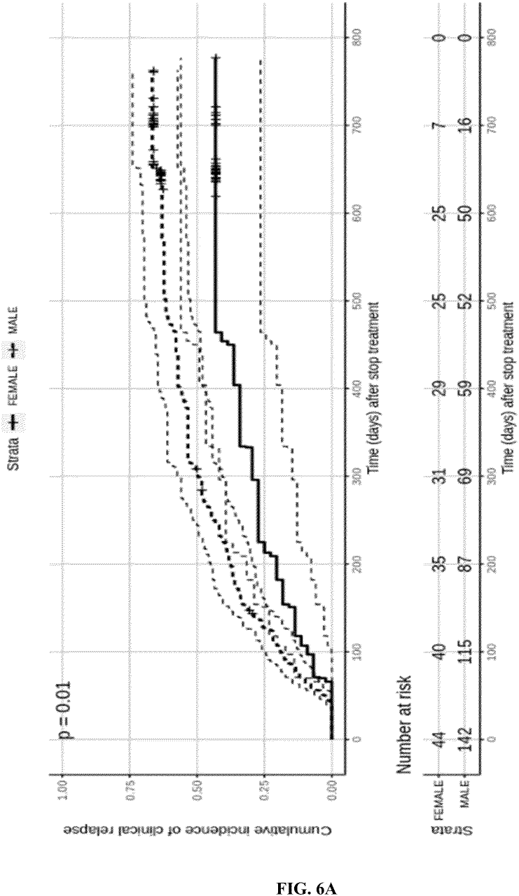

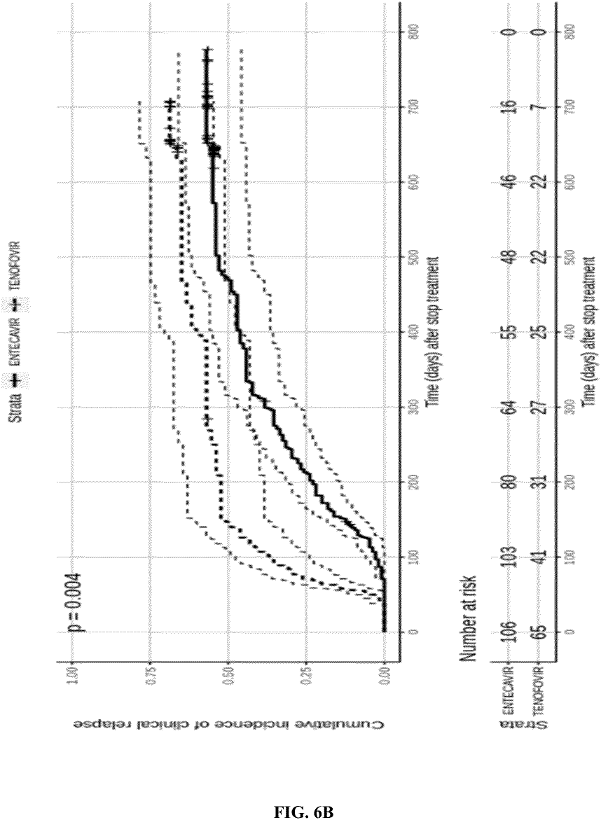

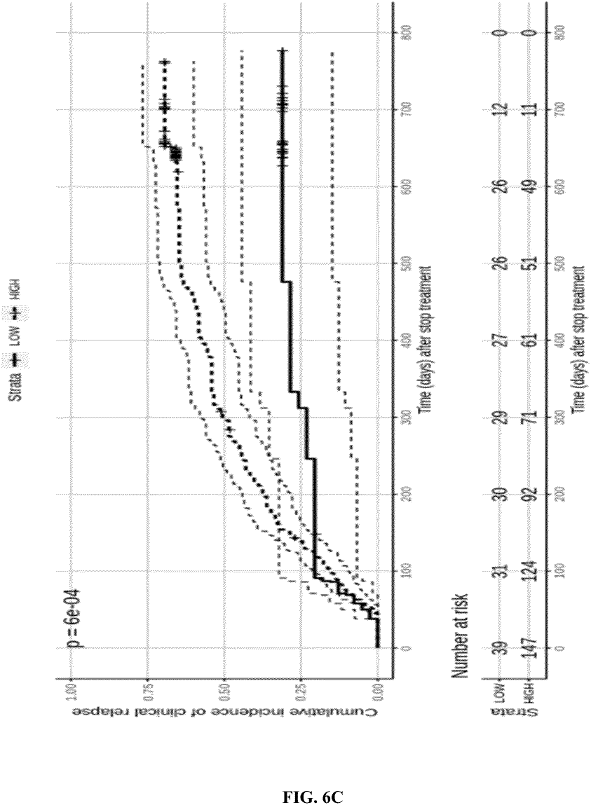

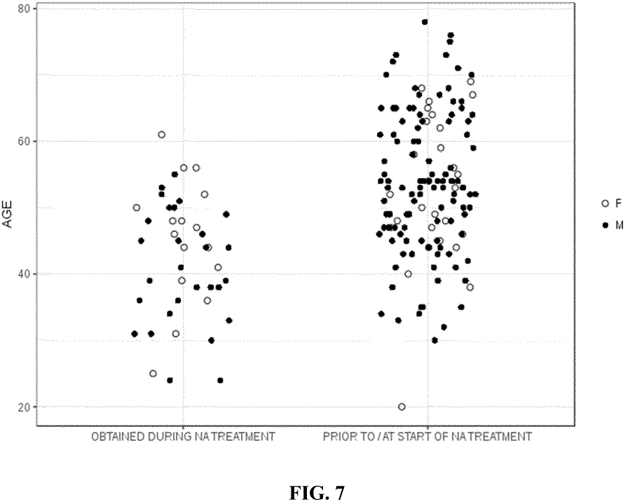

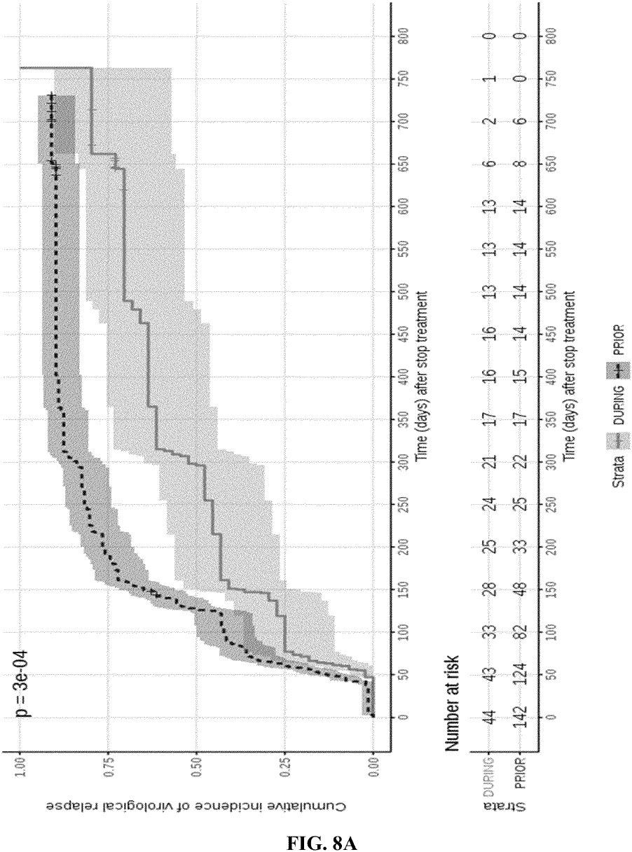

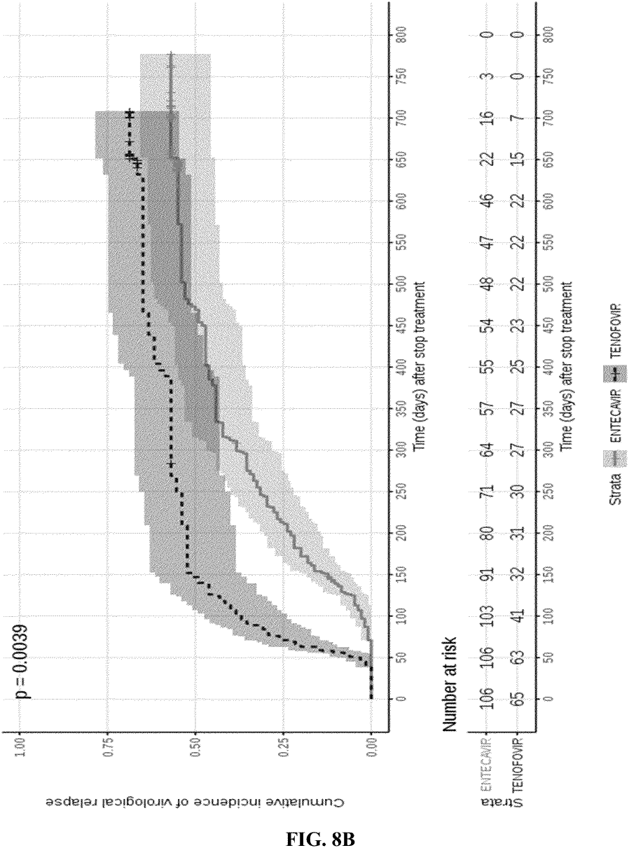

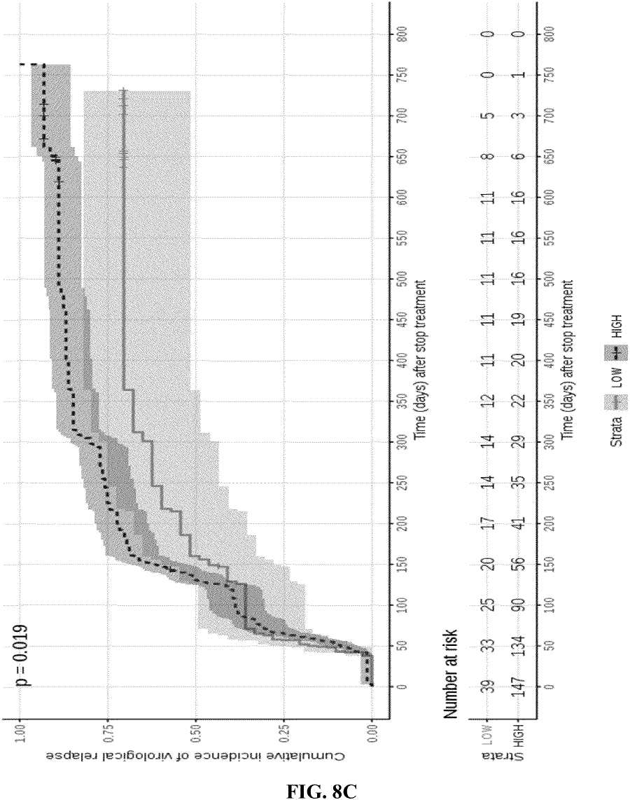

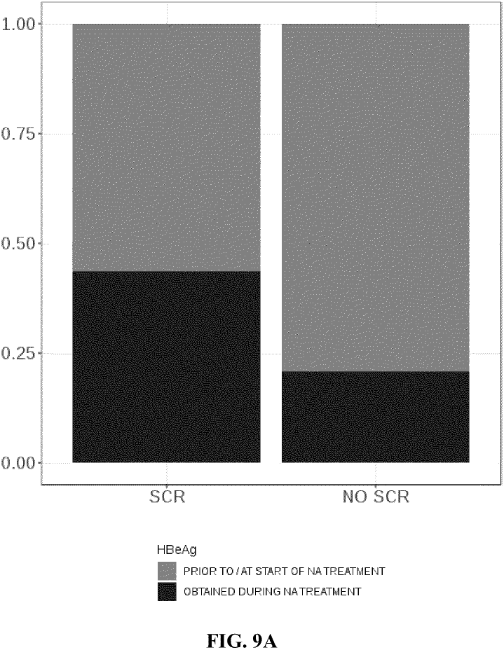

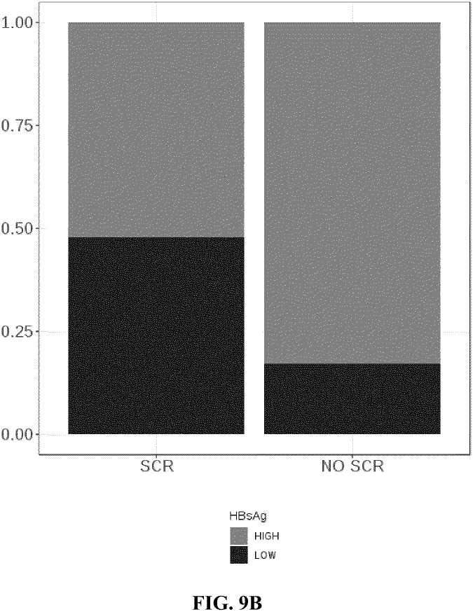

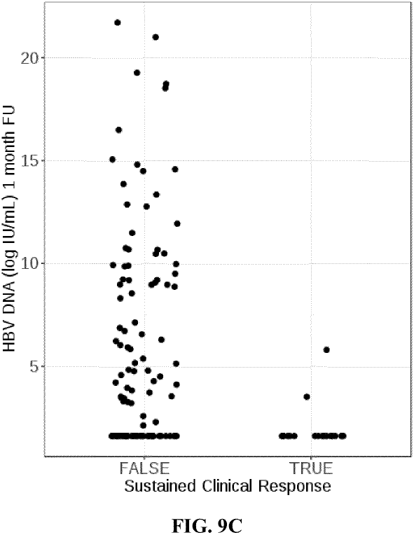

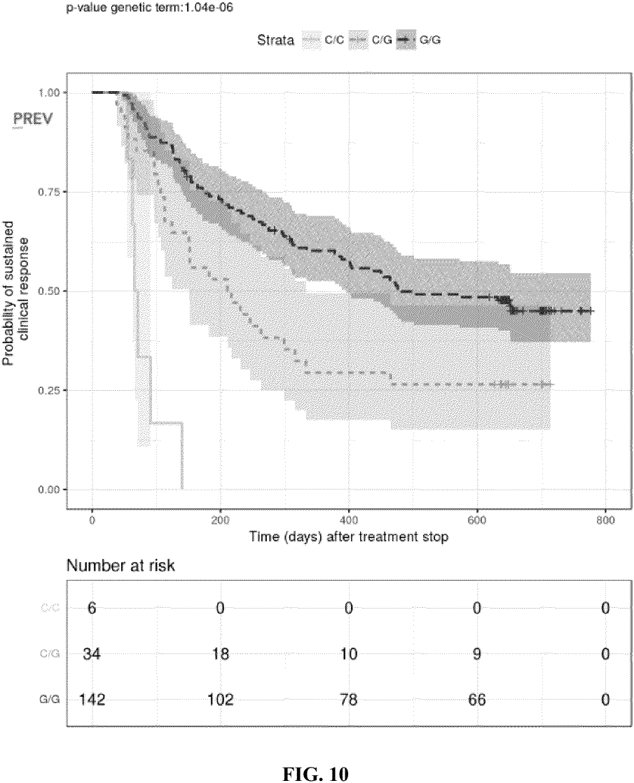

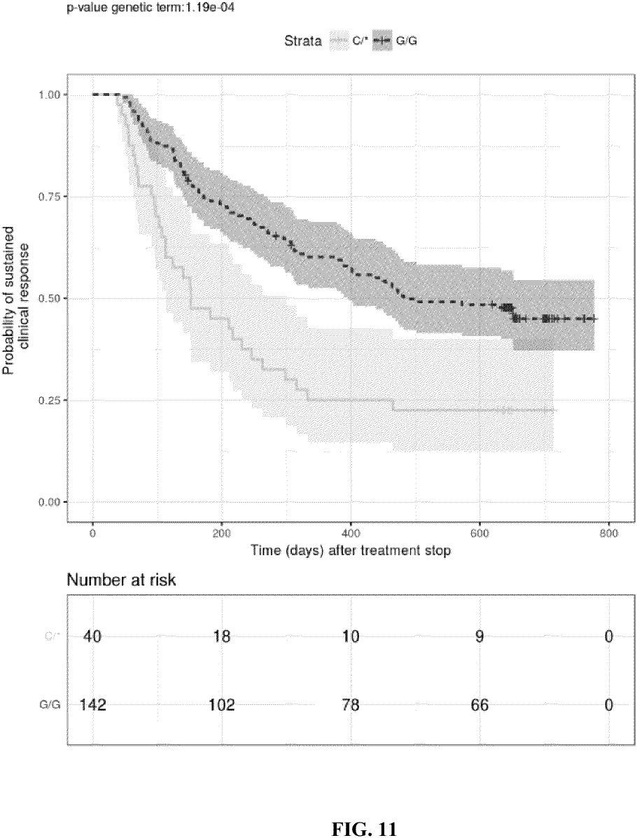

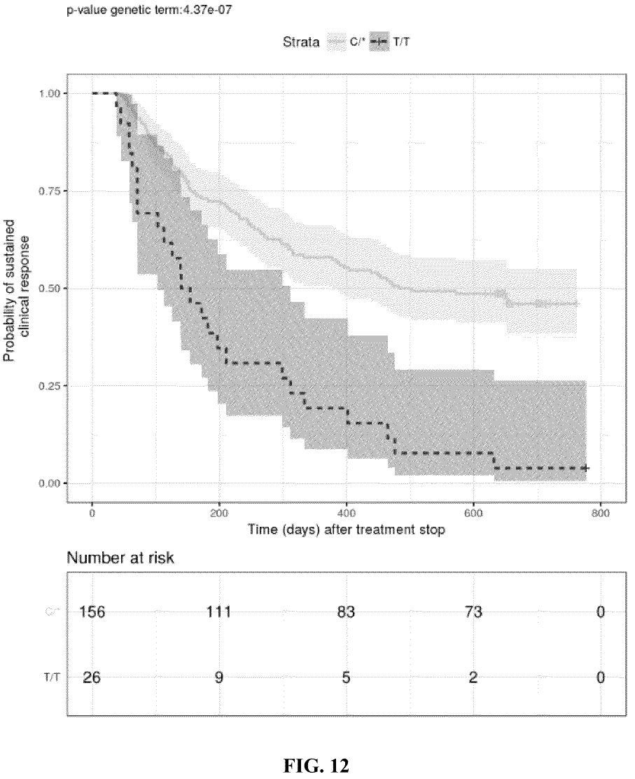

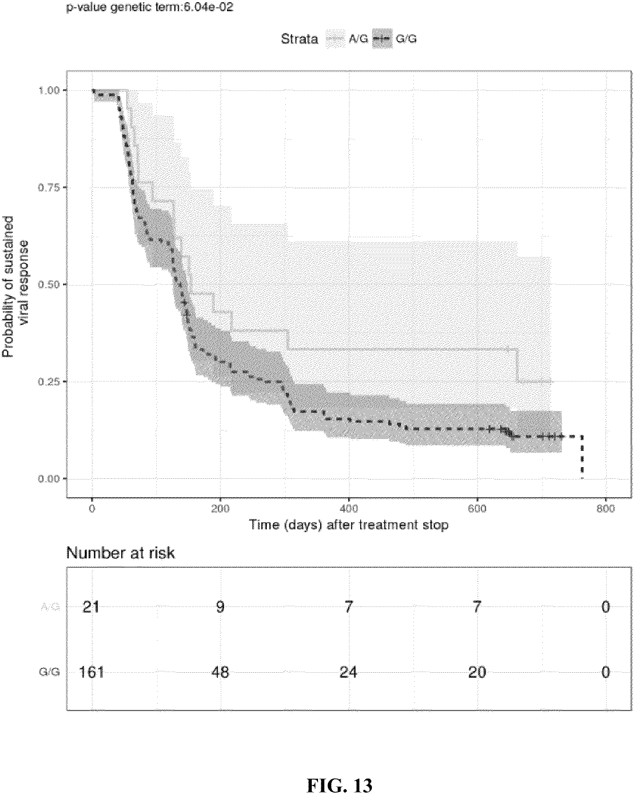

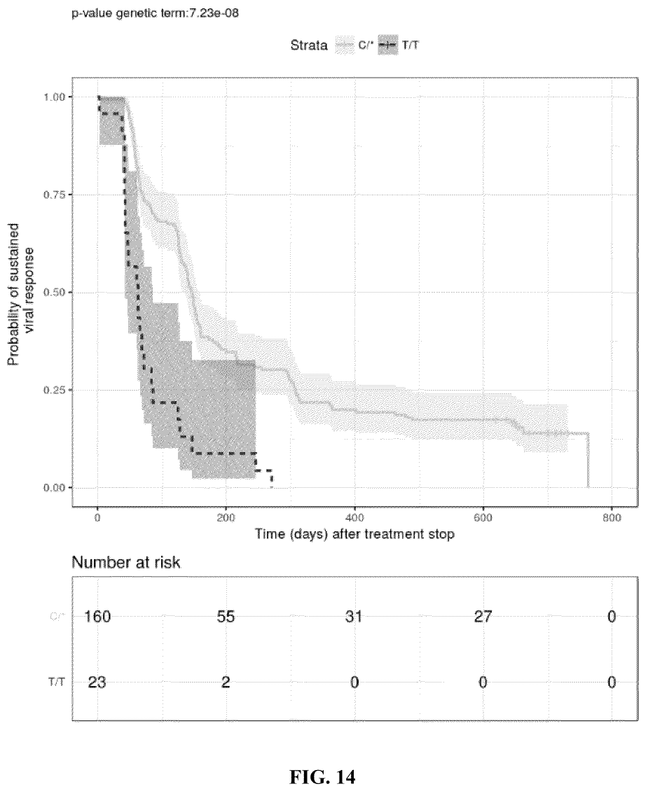

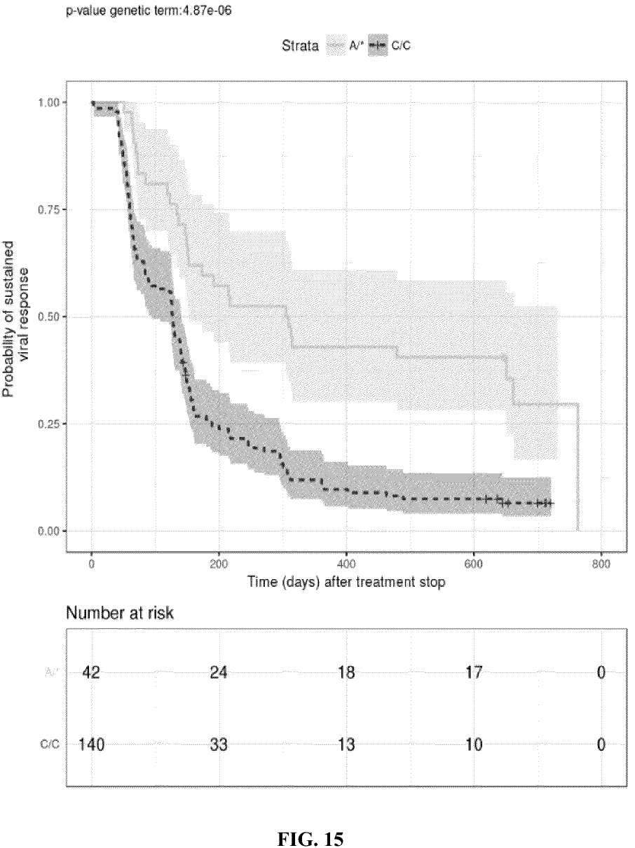

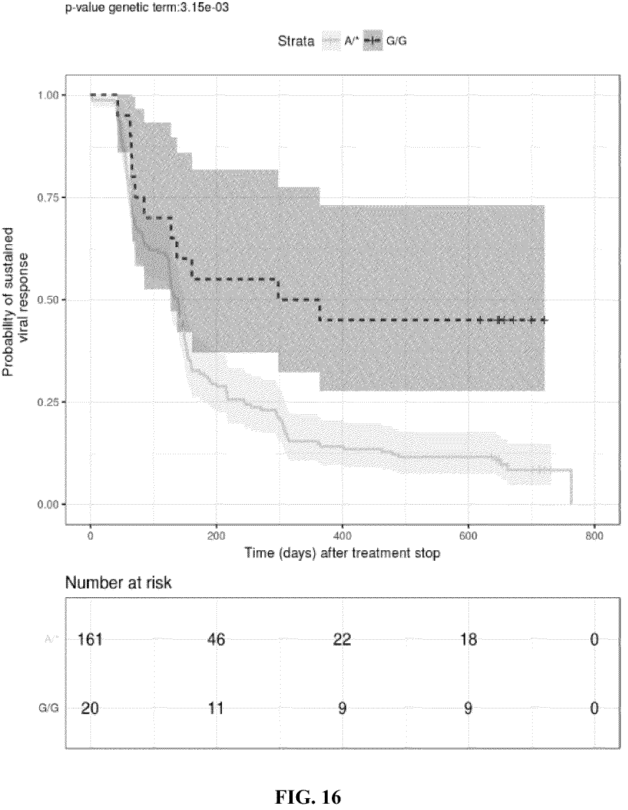

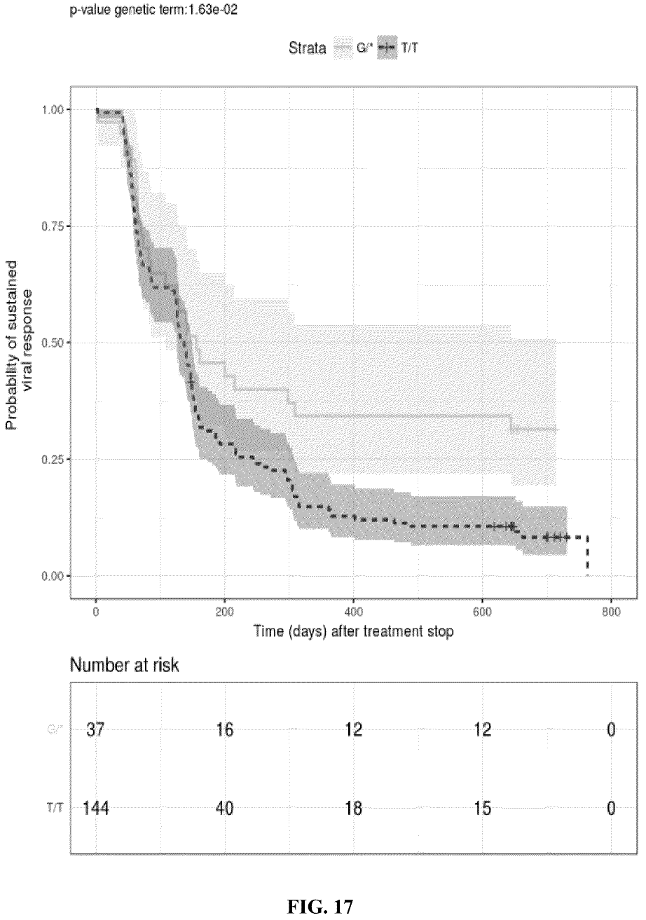

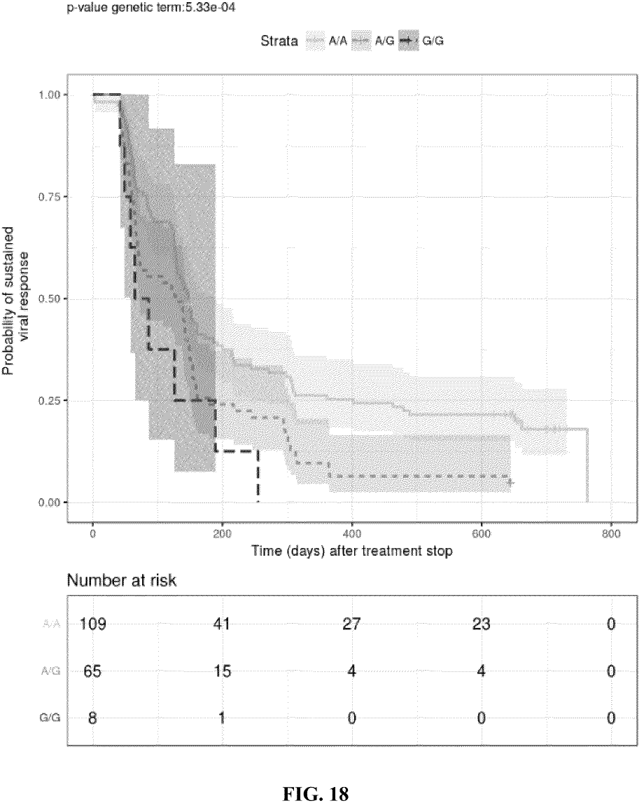

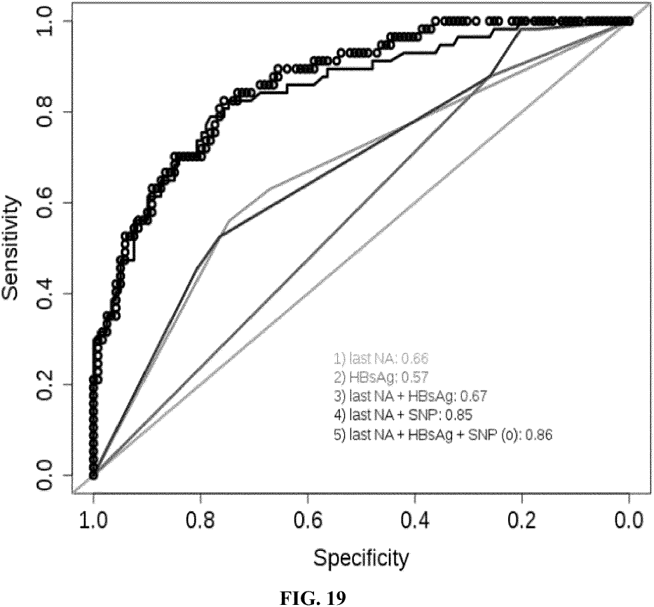

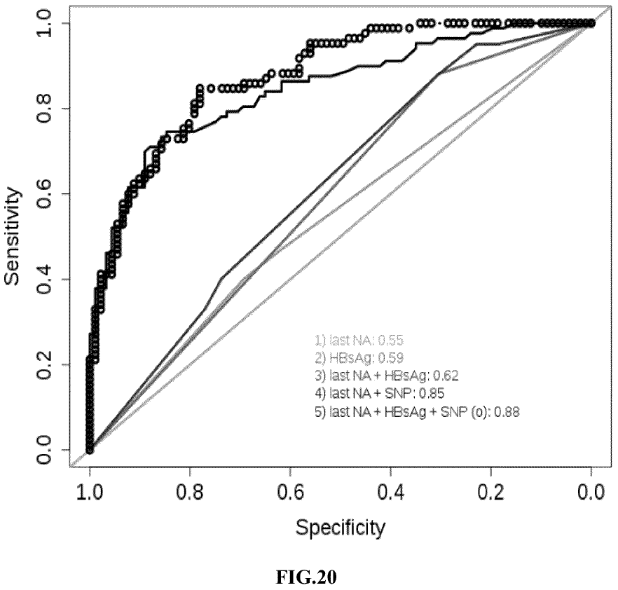

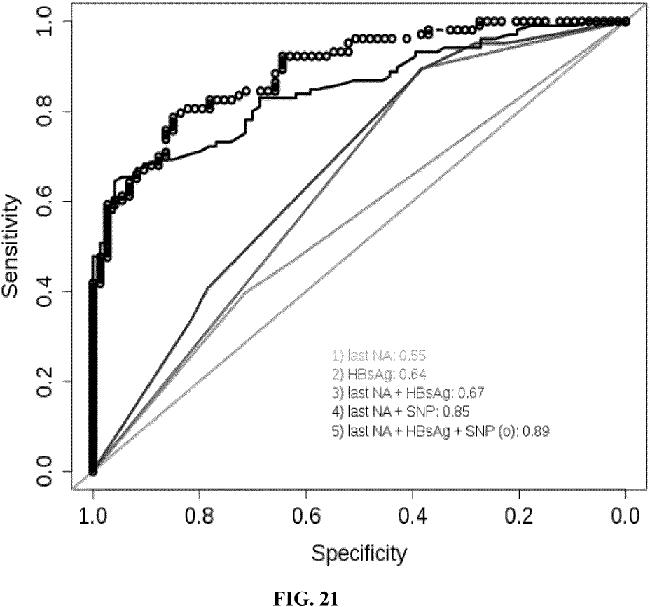

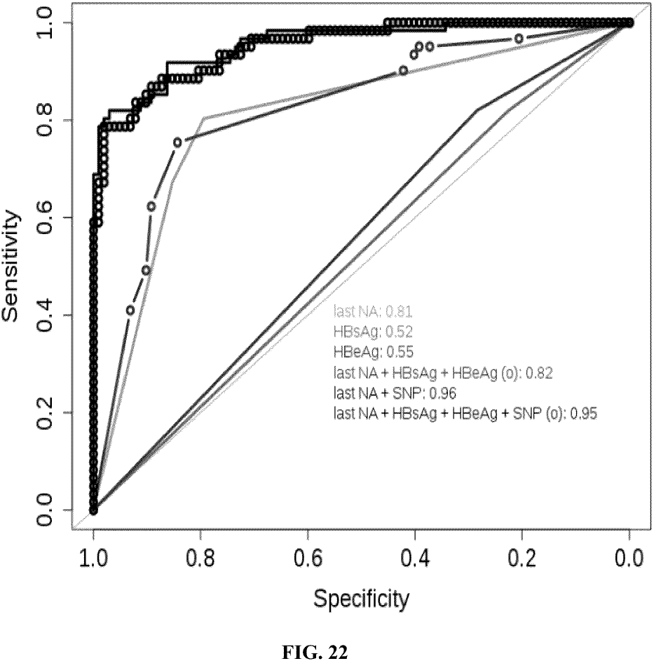

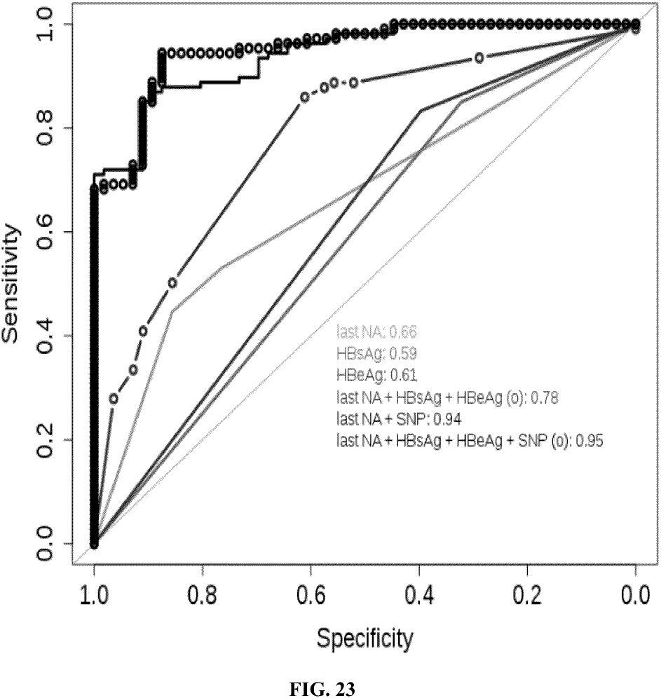

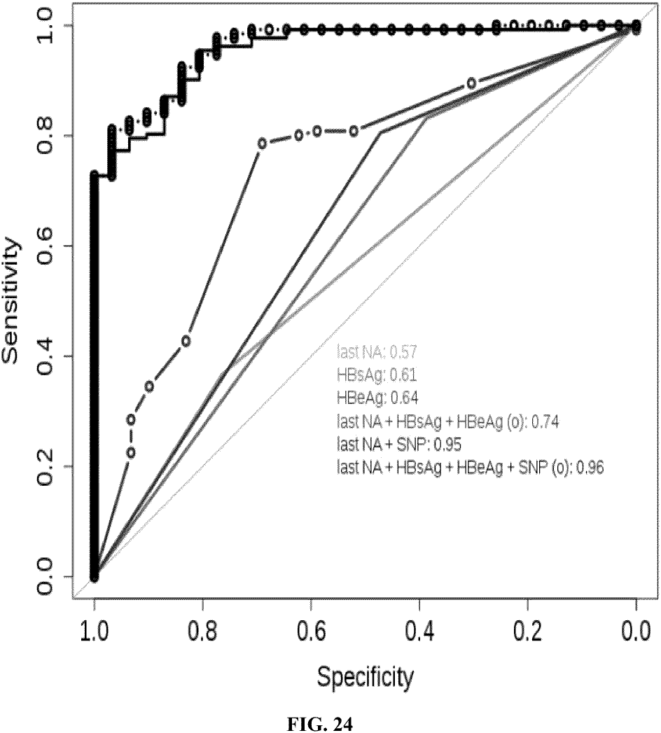

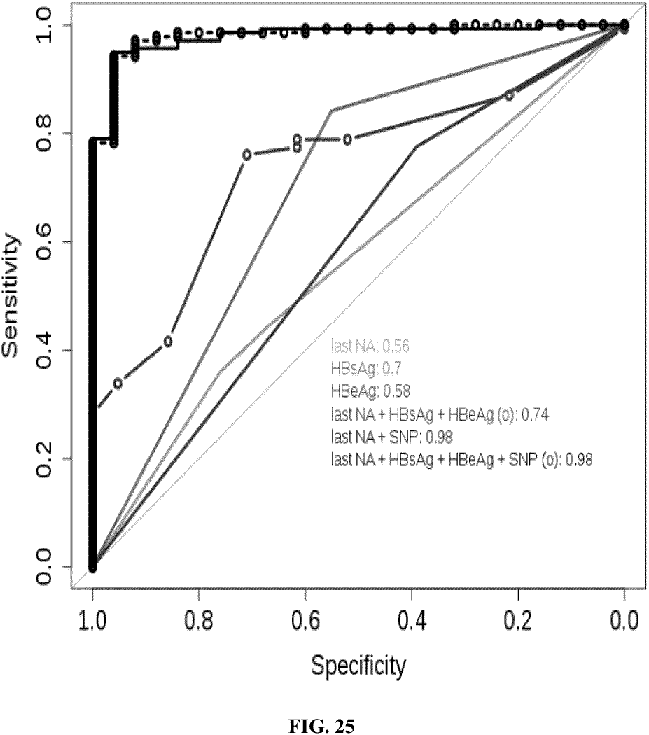

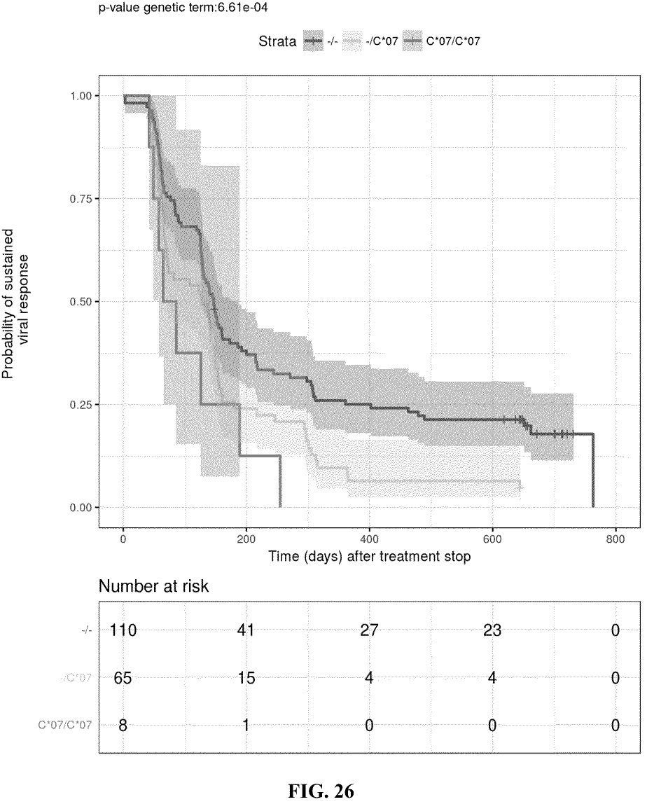

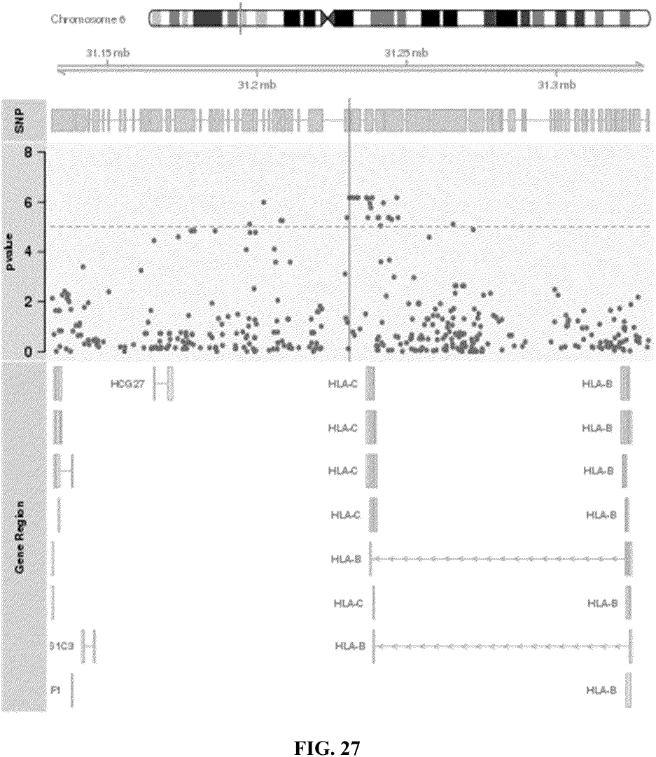

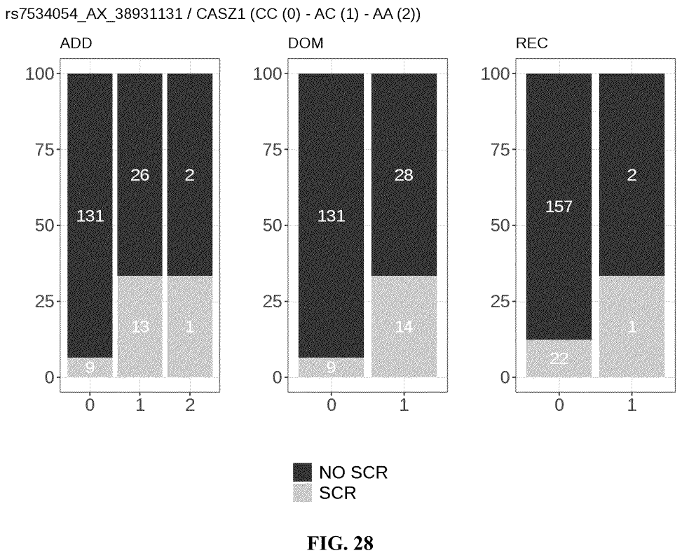

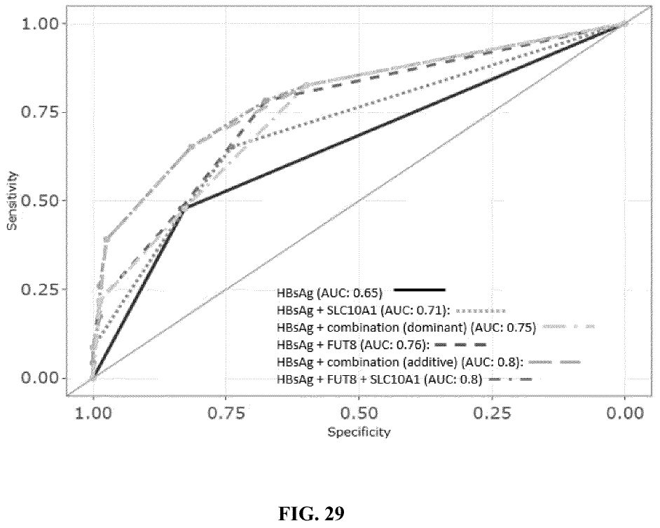

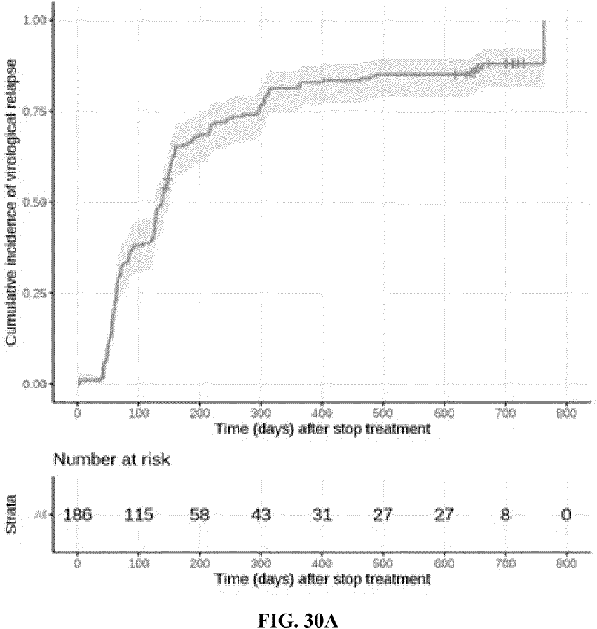

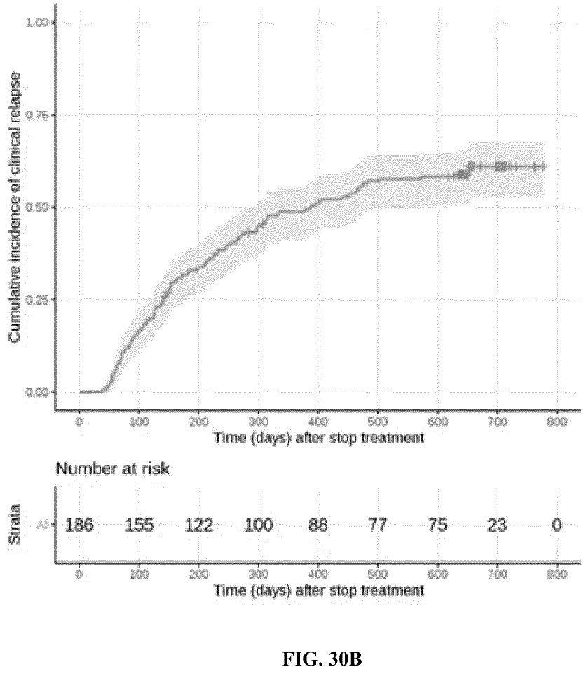

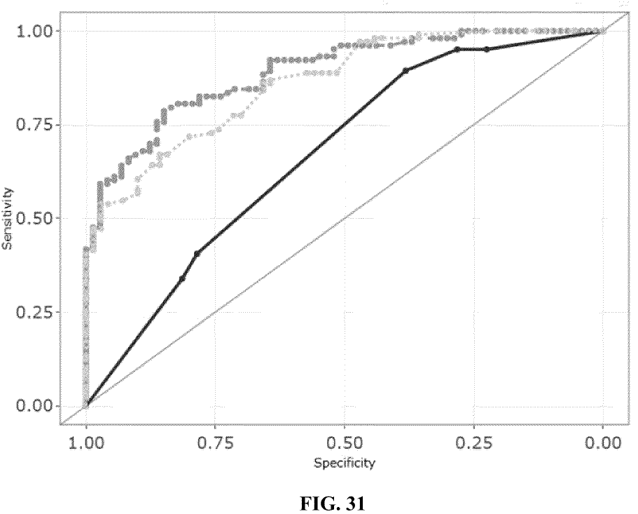

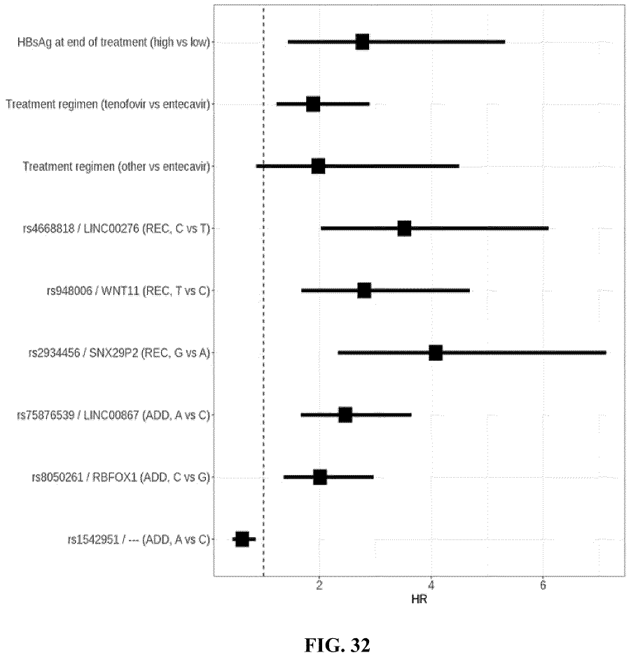

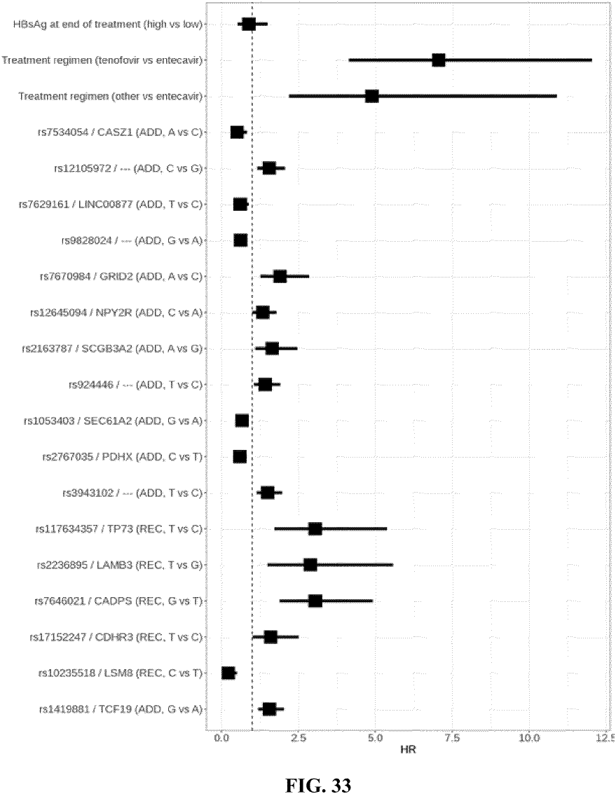

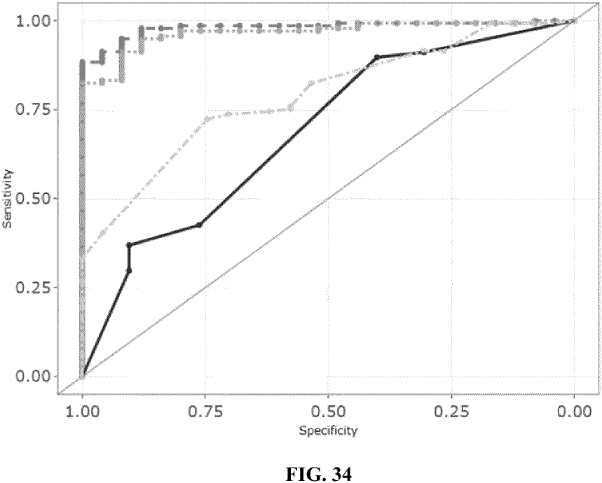

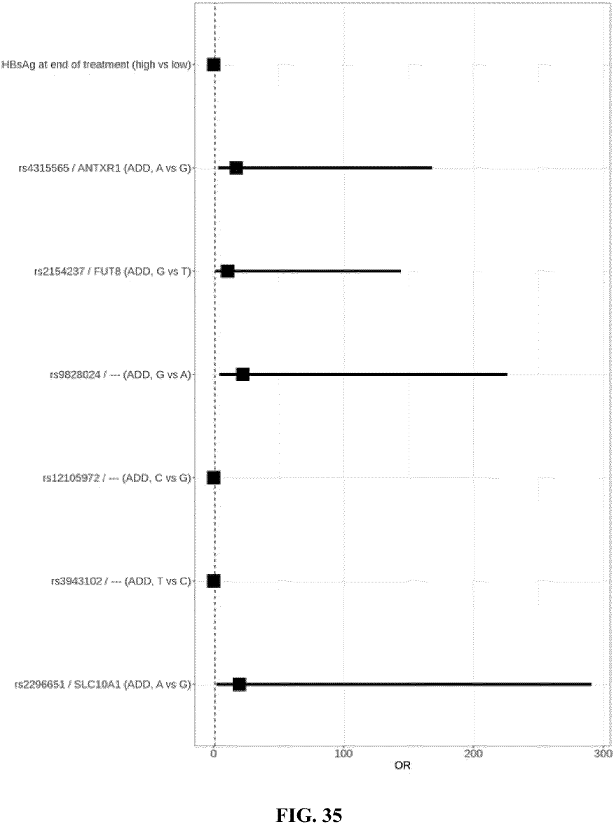

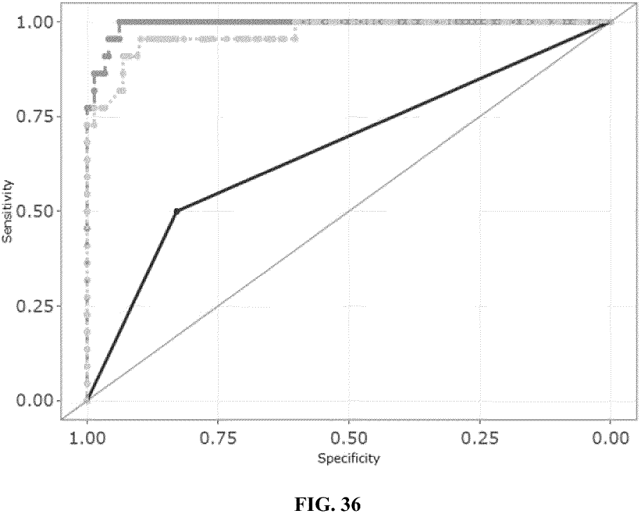

Not for all patients experiencing a virological or clinical relapse NA therapy was immediately re-initiated. More specifically, 29 clinical relapsers and 49 virological relapsers completed the study. Shown in FIGS. 2A-D are HBV DNA profiles, shaped by corresponding ALT levels, per clinical relapse and end of study event group.

Out of the 186 patients included in the cohort, 161 (86.6%) experienced a virological relapse, of whom 110 (59.2%) also had a clinical relapse. For 23 patients (12.4%), sustained clinical response was observed over the follow-up period (note that the patient who died and the patient diagnosed with hepatocellular carcinoma were excluded from the sustained clinical response group). 51 (27.42%) patients had a virological relapse without a clinical relapse. For 28 of them, the virological relapse was transient. Eight patients out of the 110 with a clinical relapse experienced a transient clinical relapse. Relapse is denoted transient if HBV DNA < 2000 IU/mL and ALT < 2x ULN is obtained at the last post-treatment observation. FIGS. 3A-H show the HBV DNA and ALT profiles for the eight transient clinical relapsers.

HBsAg loss (HBsAg < 0.05 IU/mL) was observed for 11 (5.91%) patients, 6 of them also HBsAb positive (HBsAb > 10 mIU/mL). Out of the 11 HBsAg loss patients, 5 acquired HBsAg loss already on treatment and 6 after stop of treatment (at 64, 215, 266, 299, 470, and 643 days after treatment cessation). For all latter 6 patients HBsAg levels were below 20 IU/mL at the end of treatment.

Clinical Relapse: The multivariate Cox proportional hazard regression analysis reveals that older age (HR, 1.02; 95% CI, 1.00-1.04; P = 0.02), male gender (HR, 1.71; 95% CI, 1.04-2.81; P = 0.04), an increase in the number of treatment free periods longer than 1 month (HR, 1.33; 95% CI, 1.10-1.62; P = 0.004), tenofovir treatment (compared to entecavir treatment) (HR, 1.74; 95% CI, 1.17-2.59; P = 0.007), and high HBsAg at the end of treatment (HR, 2.75; 95% CI, 1.48-5.09; P = 0.001), are negatively associated to clinical relapse (Table 4). An HBsAg level of 100 IU/mL was used as threshold to differentiate low and high end of treatment HBsAg, corresponding to 89.09% sensitivity and 35.53% specificity

TABLE 4

Cox proportional hazard regression analysis for clinical relapse

| Variable | Univariate analysis | | Multivariate analysis | |
|---|---|---|---|---|
| | HR (95% CI) | P value | HR (95% CI) | P value |
| Age (years) | 1.01 (0.99-1.02) | 0.43 | 1.02 (1.00-1.04) | 0.02 |
| Gender (male vs female) | 1.88 (1.15-3.09) | 0.01 | 1.64 (1.00-2.70) | 0.05 |
| Previous NA therapy (yes vs no) | 1.26 (0.86-1.86) | 0.24 | | |
| Number of treatment free periods > 1 month | 1.32 (1.11-1.57) | 0.002 | 1.33 (1.11-1.60) | 0.003 |
| Baseline HBeAg status (prior negativity vs during treatment) | 1.35 (0.85-2.14) | 0.20 | | |
| Treatment regimen | | | | |
| Tenofovir vs entecavir | 1.77 (1.19-2.62) | 0.005 | 1.78 (1.20-2.64) | 0.004 |
| Other vs entecavir | 1.07 (0.51-2.25) | 0.85 | 1.07 (0.50-2.29) | 0.86 |
| Length therapy (years) | 0.96 (0.86-1.06) | 0.39 | | |
| On-treatment viral suppression | | | | |
| Inconsistent vs consistent | 1.46 (0.86-2.47) | 0.16 | | |
| No vs consistent | 1.71 (0.88-3.32) | 0.12 | | |
| HBV DNA at end of treatment (detected vs not detected) | 1.39 (0.95-2.03) | 0.09 | | |
| HBsAg at end of treatment (≥ 100 IU/mL vs < 100 IU/mL) | 2.77 (1.51-5.05) | <0.001 | 2.90 (1.57-5.38) | <0.001 |
| HBsAb at end of treatment (log mIU/mL) | 0.86 (0.63-1.18) | 0.35 | | |
| ALT at end of treatment (log U/L) | 1.04 (0.70-1.53) | 0.85 | | |
| AST at end of treatment (log U/L) | 1.02 (0.54-1.91) | 0.96 | | |
| HBV DNA at 1 month after stop treatment (log IU/mL) | 1.21 (1.15-1.27) | <0.001 | | |
| ALT at 1 month after stop treatment (log U/L) | 2.19 (1.60-3.01) | <0.001 | | |
| HBsAg at 1 month after stop treatment (log IU/mL) | 1.21 (1.08-1.35) | <0.001 | | |

HBV DNA, HBsAg, and ALT at 1 month after stop treatment are additional variables showing a significant association to clinical relapse in the univariate models. These covariates were however excluded from the multivariate model as HBV DNA and ALT at 1 month after stop treatment are highly associated to the treatment regimen (FIGS. 4A-D) and HBsAg 1 month after stop treatment highly associated to end of treatment HBsAg (r = 0.93, P < 0.001, FIGS. 5A-B).

The onset of clinical relapse is on average 116 days later in female than in male, 115 days later after stopping entecavir treatment compared to tenofovir treatment, and 148 days later in the EOT HBsAg low group compared to the high group. FIGS. 6A-C show the cumulative incidence of clinical relapse after stop treatment associated to (6A) gender, (6B) treatment regimen, and (6C) HBsAg level at the end of treatment. Kaplan-Meier curves with confidence intervals and corresponding p-values of the log-rank test are presented. Finally, note that the number of treatment free periods > 1 month is significantly correlated to clinical relapse time (r = -0.20, P = 0.006).

Virological Relapse: The multivariate Cox proportional hazard regression analysis reveals that an increase in the number of treatment free periods longer than 1 month (HR, 1.83; 95% CI, 1.52-2.20; P < 0.001), prior HBeAg negativity at the start of NA therapy (compared to acquiring HBeAg negativity during treatment) (HR, 2.35; 95% CI, 1.57-3.52; P < 0.001), tenofovir treatment (compared to entecavir treatment) (HR, 3.02; 95% CI, 2.11-4.32; P < 0.001), and high HBsAg at the end of treatment (HR, 1.93; 95% CI, 1.25-2.98; P = 0.003), are negatively associated to virological relapse (Table 5).

TABLE 5

Cox proportional hazard regression analysis for virological relapse

| Variable | Univariate analysis | | Multivariate analysis | |
|---|---|---|---|---|
| | HR (95% CI) | P value | HR (95% CI) | P value |
| Age (years) | 1.01 (1.00-1.03) | 0.05 | | |
| Gender (male vs female) | 1.53 (1.05-2.23) | 0.03 | | |
| Previous NA therapy (yes vs no) | 1.64 (1.19-2.27) | 0.003 | | |
| Number of treatment free periods > 1 month | 1.66 (1.40-1.97) | <0.001 | 1.83 (1.53-2.20) | <0.001 |
| Baseline HBeAg status (prior negativity vs during treatment) | 2.02 (1.37-2.98) | <0.001 | 2.35 (1.57-3.52 | 0.001 |
| Treatment regimen | | | | |
| Tenofovir vs entecavir | 2.49 (1.77-3.49) | <0.001 | 3.02 (2.11-4.32 | <0.001 |
| Other vs entecavir | 1.57 (0.88-2.81) | 0.13 | 2.69 (1.45-4.99) | 0.002 |
| Length NA therapy (years) | 0.99 (0.91-1.07) | 0.79 | | |
| On-treatment viral suppression | | | | |
| Inconsistent vs consistent | 0.94 (0.63-1.41) | 0.77 | | |
| No vs consistent | 1.17 (0.69-2.00) | 0.56 | | |
| HBV DNA at end of treatment (detected vs not detected) | 1.08 (0.79-1.47) | 0.64 | | |
| HBsAg at end of treatment (≥ 100 IU/mL vs < 100 IU/mL) | 1.64 (1.08-2.49) | 0.02 | 1.93 (1.25-2.98) | 0.003 |
| HBsAb at end of treatment (log mIU/mL) | 0.92 (0.73-1.16) | 0.49 | | |
| ALT at end of treatment (log U/L) | 1.09 (0.81-1.48) | 0.57 | | |
| AST at end of treatment (log U/L) | 1.03 (0.62-1.70) | 0.93 | | |
| HBV DNA at 1 month after stop treatment (log IU/mL) | 1.21 (1.16-1.26) | <0.001 | | |
| ALT at 1 month after stop treatment (log U/L) | 1.55 (1.23-1.97) | <0.001 | | |
| HBsAg at 1 month after stop treatment (log IU/mL) | 1.08 (1.01-1.16) | 0.03 | | |

HBV DNA, HBsAg, and ALT at 1 month after treatment cessation were again excluded from the multivariate model due to strong association to the treatment regimen and end of treatment HBsAg. In addition, the presence or absence of previous NA therapy is obviously related to the number of treatment free periods (P < 0.001) and therefore excluded from the multivariate model. Finally, baseline HBeAg is associated to age (P < 0.001) and gender (P = 0.03), which is illustrated in FIG. 7, and therefore also excluded from the multivariate model.

The onset of virological relapse is on average 153 days later in patients obtaining HBeAg negativity during NA treatment compared to patients HBeAg negative prior to start of treatment, 119 days later after stopping entecavir treatment compared to tenofovir treatment, and 88 days later in the EOT HBsAg low group compared to the high group. FIGS. 8A-C shows the cumulative incidence of virological relapse after stop treatment associated to (8A) prior HBeAg status, (8B) treatment regimen, and (8C) HBsAg level at the end of treatment. Kaplan-Meier curves with confidence intervals and corresponding p-values of the log-rank test are presented. Finally, note that the number of treatment free periods > 1 month is significantly correlated to viral relapse time (r = -0.27, P < 0.001).

Sustained Clinical Response: Logistic regression shows the negative association of prior HBeAg negativity at the start of NA therapy (OR, 0.25; 95% CI, 0.06-0.99; P = 0.05; FIG. 9A), high HBsAg at the end of treatment (OR, 0.16; 95% CI, 0.04-0.55; P = 0.005, FIG. 9B), and higher HBV DNA 1 month after stop treatment (OR, 0.54; 95% CI, 0.28-0.78; P = 0.01, FIG. 9C), to sustained clinical response (Table 6).

The average HBsAg at the end of treatment among the 23 sustained clinical responders is 195 IU/mL, with values ranging from 0.05 IU/mL to 32630 IU/mL.

TABLE 6

Logistic regression analysis for sustained clinical response

| Variable | Univariate analysis | | Multivariate analysis | |
|---|---|---|---|---|
| | OR (95% CI) | P value | OR (95% CI) | P value |
| Age (years) | 0.98 (0.94-1.01) | 0.21 | | |
| Gender (male vs female) | 0.43 (0.17-1.10) | 0.07 | | |
| Previous NA therapy (yes vs no) | 0.64 (0.22-1.63) | 0.37 | | |
| Number of treatment free periods > 1 month | 0.34 (0.09-0.80) | 0.05 | | |
| Baseline HBeAg status (prior negativity vs during treatment) | 0.34 (0.14-0.87) | 0.02 | 0.25 (0.06-0.99) | 0.05 |
| Treatment regimen | | | | |
| Tenofovir vs entecavir | 0.79 (0.29-2.03) | 0.64 | | |
| Other vs entecavir | 1.01 (0.15-4.20) | 0.99 | | |
| Length NA therapy (years) | 1.01 (0.79-1.24) | 0.93 | | |
| On-treatment viral suppression | | | | |
| Inconsistent vs consistent | 0.81 (0.30-2.40) | 0.68 | | |
| No vs consistent | 0.21 (0.01-1.36) | 0.16 | | |
| HBV DNA at end of treatment (detected vs not detected) | 0.69 (0.28-1.65) | 0.41 | | |
| HBsAg at end of treatment (≥ 100 IU/mL vs < 100 IU/mL) | 0.23 (0.09-0.57) | 0.001 | 0.16 (0.04-0.55) | 0.005 |
| HBsAb at end of treatment (log mIU/mL) | 1.32 (0.78-2.04) | 0.22 | | |
| ALT at end of treatment (log U/L) | 1.40 (0.55-3.59) | 0.48 | | |
| AST at end of treatment (log U/L) | 1.34 (0.29-5.79) | 0.70 | | |
| HBV DNA at 1 month after stop treatment (log IU/mL) | 0.53 (0.27-0.77) | 0.01 | 0.54 (0.28-0.78) | 0.01 |
| ALT at 1 month after stop treatment (log U/L) | 0.59 (0.25-1.21) | 0.20 | | |
| HBsAg at 1 month after stop treatment (log IU/mL) | 0.72 (0.59-0.86) | <0.001 | | |

Genetic Profiling - Candidate Gene Approach: From the analysis of the twenty two candidate genes (Table 1), two genes (CTLA4, SNP rs231770 and HLA-DPB1, SNP rs9277535) were found to be associated with time to clinical relapse, and four genes (HLA-C, SNP rs3130542; STAT4, SNP rs7574865; SLC10A1, SNP rs2296651; and TCF19, SNP rs1419881) were found to be associated with time to viral relapse. See Table 7.

TABLE 7

Hit SNPs from Candidate Genes

| RSID | GENE | GENETIC MODEL | ESTIMATE | PVALUE | MAJOR ALLELE | MINOR ALLELE | RISK FACTOR | PROTECTIVE FACTOR | RESPONSE |
|---|---|---|---|---|---|---|---|---|---|
| rs231770 | CTLA4 | Additive | -0.29996 | 0.0346503 | T | C | Presence of T allele | Presence of C allele | Time to CR |
| rs9277535 | HLA-DPB1 | Additive | -0.37286 | 0.0371615 | G | A | Presence of G allele | Presence of A allele | Time to CR |
| rs3130542 | HLA-C | Additive | 0.72804 | 6.66E-07 | G | A | Presence of A allele | Presence of G allele | Time to VR |
| rs7574865 | STAT4 | Dominant | -0.32362 | 0.0484403 | G | T | Presence of GG genotype | Presence of T allele | Time to VR |
| rs2296651 | SLC10A1 | Additive | -0.6226 | 0.0240025 | G | A | Presence of G allele | Presence of A allele | Time to VR |
| rs1419881 | TCF19 | Additive | 0.26178 | 0.02037 | A | G | Presence of G allele | Presence of A allele | Time to VR |

For example, after adjustment for last NUC therapy, SNP rs231770 (genotype C/C) of CTLA4 (p=0.01) and SNP r2296651 (genotype A/G) of SLC10A1 (FIG. 13) were found to be protective for clinical relapse and viral relapse, respectively; SNP rs9277535 (G/G) of HLA-DPB1 (p=0.04) was found to be associated with clinical relapse; and no significant association with clinical relapse was found with each of SNPs rs3077 of HLA-DPA1, rs9366816 of HLA-DPA3, rs9276370 of HLA-DQA2, rs7756516 and rs7453920 of HLA-DQB2.

Genetic Profiling - Genome-wide Association (GWAS): Seven new SNPs were identified to be significantly associated with time to clinical relapse (Table 8).

TABLE 8

New SNPs Associated with Time to Clinical Relapse

| RSID | GENE | GENETIC MODEL | ESTIMATE | PVALUE | MAJOR ALLELE | MINOR ALLELE | RISK FACTOR | PROTECTIVE FACTOR | RESPONSE |
|---|---|---|---|---|---|---|---|---|---|
| rs4668818 | LINC00276 | Recessive | 1.35383 | 8.47E-07 | T | C | Presence of CC genotype | Presence of T allele | Time to CR |
| rs948006 | WNT11 | Recessive | 1.18847 | 4.25E-07 | C | T | Presence of TT genotype | Presence of C allele | Time to CR |
| rs2934456 | SNX29P2 | Recessive | 1.35077 | 2.68E-07 | A | G | Presence of GG genotype | Presence of A allele | Time to CR |
| rs77586835 | --- | Additive | 0.83099 | 3.30E-06 | T | C | Presence of C allele | Presence of T allele | Time to CR |
| rs75876539 | LINC00867 | Additive | 0.86924 | 2.09E-06 | C | A | Presence of A allele | Presence of C allele | Time to CR |
| rs8050261 | RBFOX1 | Additive | 0.85877 | 2.95E-06 | G | C | Presence of C allele | Presence of G allele | Time to CR |
| rs1542951 | --- | Additive | -0.66951 | 4.89E-06 | C | A | Presence of C allele | Presence of A allele | Time to CR |

For example, SNP rs8050261 (genotype A/A) of RBFOX1 (FIGS. 10 and 11) and SNP rs948006 (allele C) of WNT11 (FIG. 12) were found to be protective for clinical relapse.

Twenty nine new SNPs were identified be significantly associated with time to virological relapse (Table 9).

TABLE 9

New SNPs Associated with Time to Virological Relapse

| RSID | GENE | GENETIC MODEL | ESTIMATE | PVALUE | MAJOR ALLELE | MINOR ALLELE | RISK FACTOR | PROTECTIVE FACTOR | RESPONSE |
|---|---|---|---|---|---|---|---|---|---|
| rs7534054 | CASZ1 | Additive | -1.04099 | 7.62E-07 | C | A | Presence of C allele | Presence of A allele | Tune to VR |
| rs4315565 | ANTXR1 | Additive | -0.63805 | 2.96E-06 | G | A | Presence of G allele | Presence of A allele | Time to VR |
| rs12105972 | --- | Additive | 0.54109 | 5.40E-06 | G | C | Presence of C allele | Presence of G allele | Time to VR |
| rs1994245 | TMEFF2 | Additive | 0.5708 | 2.78E-06 | T | C | Presence of C allele | Presence of T allele | Time to VR |
| rs11896590 | TMEFF2 | Additive | -0.53833 | 4.77E-06 | A | G | Presence of A allele | Presence of G allele | Time to VR |
| rs7629161 | LLYC00877 | Additive | -0.81837 | 4.41E-07 | C | T | Presence of C allele | Presence of T allele | Time to VR |
| rs9825024 | --- | Additiive | -0.63531 | 3.51E-07 | A | G | Presence of A allele | Presence of G allele | Time to VR |
| rs7670984 | GRID2 | Additive | 0.78041 | 5.36E-06 | C | A | Presence of A allele | Presence of C allele | Time to VR |
| rs12645094 | NPF2R | Additive | 0.59411 | 2.41E-06 | A | C | Presense of C allele | Presence at A allele | Time to VR |
| rs2163787 | SCGB3A2 | Additive | 0.84676 | 1.94E-06 | G | A | Presence of A allele | Presence of G allele | Time to VR |
| rs924446 | --- | Additive | 0.56138 | 2.56E-06 | C | T | Presence of T allele | Presence of C allele | Time to VR |
| rs180001 | ATXV1 | Additive | -0.6157 | 1.32E-06 | A | G | Presence of A allele | Presence of G allele | Time to VR |
| rs12199613 | BTN3A2 | Additive | 0.68844 | 9.98E-07 | C | T | Presence of T allele | Presence of C allele | Time to VR |
| rs2394952 | HLA-C | Additive | 0.72804 | 6.66E-07 | A | G | Presence of G allele | Presence of A allele | Time to VR |
| rs17152258 | CDHR3 | Additive | 0.68058 | 4.49E-06 | C | T | Presence of T allele | Presence of C allele | Time to VR |
| rs7459445 | --- | Additive | 0.9052 | 8.94E-07 | T | C | Presence of C allele | Presence of T allele | Time to VR |
| rs1053403 | SEC61A2 | Additive | -0.5467 | 2.55E-06 | A | G | Presence of A allele | Presence of G allele | Time to VR |
| rs2767035 | PDHX | Additive | -0.64992 | 3.22E-07 | T | C | Presence of T allele | Presence of C allele | Time to VR |
| rs3943702 | --- | Additive | 0.59123 | 1.69E-06 | C | T | Presence of T allele | Presence of C allele | Time to VR |
| rs2154237 | FUT3 | Additive | -1.05463 | 1.65E-06 | T | G | Presence of T allele | Presence of G allele | Tme to VR |
| rs73371840 | C14orj80 | Additive | -0.565 | 5.20E-06 | T | C | Presence of T allele | Presence of C allele | Time to VR |
| rs7205040 | PRKCB | Additive | -0.64123 | 4.86E-06 | C | T | Presence of C allele | Presence of T allele | Time to VR |
| rs552219 | LAMA1 | Additive | -0.75739 | 1.25E-06 | G | A | Presence af G allele | Presence of A allele | Time to VR |
| rs78045374 | --- | Additive | 0.75648 | 3.47E-06 | T | C | Presence of C allele | Presence of T allele | Time to VR |
| rs117634357 | TP73 | Recessive | 1.26001 | 1.15E-07 | C | T | Presence of TT genotype | Presence of C allele | Time to VR |
| rs2236895 | LAMB3 | Recessive | 1.29814 | 5.26E-07 | G | T | Presence of TT genotype | Presence of G allele | Time to VR |
| rs7646021 | CADP3 | Recessive | 1.01408 | 5.35E-07 | T | G | Presence of GG genotype | Presence of T allele | Time to VR |
| rs17152247 | CDHR3 | Recessive | 0.97425 | 4.50E-07 | C | T | Presence of TT genotype | Presence of C allele | Time to VR |
| rs10235538 | LSM3 | Recessive | -1.69479 | 9.99E-07 | T | C | Presence of TT genotype | Presence of C allele | Time to VR |

For example, SNP rs117634357 of TP73 (allele C) (FIG. 14), SNP rs7534054 of CASZ1 (allele A) (FIG. 15), SNP rs180001 of ATXN1 (genotype G/G) (FIG. 16), SNP rs2154237 of FUT8 (allele G) (FIG. 17), and SNP rs2394952 (genotype A/A) were found to be protective for virological relapse.

HLA Approach: The HLAC*07 region identified by HLA-typing is another platform/approach to characterize HLA regions. This example showed that either counting the number of HLA-C*07 allele by sanger sequencing or looking at specific SNPs in HLA-C region (rs2394952 being the most significant one but all SNPs in the same linkage disequilibrium block) carried the same information. It could be considered as a technical validation. The HLA-C region has already been described in the literature as a marker for susceptibility to infection, not associated with viral relapse. FIG. 26 represents the HLA-C*07 association captured by Sanger sequencing, and FIG. 27 presents a regional plot showing that some other SNPS in close proximity (same LD block) show the same signal.

Merge With Genetic Data: For both clinical and virological relapse, the significant clinical covariates (Table 4 and Table 5) are merged with the corresponding significant hits from the GWAS analysis. Cox proportional regression analysis with lasso regularization is applied to assess the combined association of the clinical and genetic covariates to relapse. Note that a threshold was set to the absolute values of the coefficients to be included in the final Cox model, equal to 0.35 and 0.2 for clinical and virological relapse respectively. The results are shown in Table 10 and Table 11.

TABLE 10

Lasso regression model including clinical and genetic data for time to clinical relapse analysis

| Covariate | Mean | SD |
|---|---|---|
| Age (years) | 1.002283 | 0.001337 |
| Gender (male vs female) | 1.439837 | 0.060237 |
| Number of treatment free periods > 1 month | 1.00422 | 0.004347 |
| Treatment regimen (Tenofovir vs entecavir) | 1.492241 | 0.058673 |
| Treatment regimen (Other vs entecavir) | 1.259882 | 0.13406 |
| HBsAg at end of treatment (≥ 100 IU/mL vs < 100 IU/mL) | 2.109857 | 0.140677 |
| rs4668818 | 2.714697 | 0.116468 |
| rs948006 | 2.150538 | 0.039575 |
| rs2934456 | 3.327037 | 0.163316 |
| rs77586835 | 1.403124 | 0.030484 |
| rs75876539 | 1.801101 | 0.039125 |
| rs8050261 | 1.771477 | 0.035107 |
| rs1542951 | 0.717347 | 0.005239 |
| rs231770 | 0.863401 | 0.025239 |
| rs9277535 | 0.812151 | 0.014801 |

The lasso model was repeatedly fitted across 1000 iterations, using cross validation to find the optimal value for the penalty parameter. In case a coefficient was set to zero in all iterations, the covariate is excluded. The table below provides the mean and standard deviation of the estimated hazard ratios across the 1000 iterations.

TABLE 11

Lasso regression model including clinical and genetic data for time to virological relapse analysis

| | | |
|---|---|---|
| Covariate | Mean | SD |
| Age (years) | N/A | N/A |
| Number of treatment free periods > 1 month | 1.126237 | 0.000345 |
| Baseline HBeAg status (prior negativity vs during treatment) | 1.331564 | 0.035316 |
| Treatment regimen (Tenofovir vs entecavir) | 2.662436 | 0.163749 |
| Treatment regimen (Other vs entecavir) | 1.187754 | 0.109529 |
| HBsAg at end of treatment (≥ 100 IU/mL vs < 100 IU/mL) | 1.000016 | 0.0000728 |
| rs7534054 | 0.67876 | 0.002466 |
| rs4315565 | 0.888783 | 0.004982 |
| rs12105972 | 1.102381 | 0.006513 |
| rs1994245 | 1.11012 | 0.003044 |
| rs11896590 | N/A | N/A |
| rs7629161 | 0.917728 | 0.015243 |
| rs9828024 | 0.833292 | 0.006421 |
| rs7670984 | 1.431911 | 0.022985 |
| rs12645094 | 1.143157 | 0.014594 |
| rs2163787 | 1.339706 | 0.02746 |
| rs924446 | 1.261534 | 0.016174 |
| rs180001 | 0.909172 | 0.003583 |
| rs12199613 | 1.248125 | 0.010729 |
| rs2394952 | 1.033823 | 0.013213 |
| rs17152258 | 1.069923 | 0.011019 |
| rs7459445 | 1.093129 | 0.013485 |
| rs1053403 | 0.877004 | 0.007734 |
| rs2767035 | 0.793115 | 0.0061 |
| rs3943102 | 1.05163 | 0.002701 |
| rs2154237 | N/A | N/A |
| rs73371840 | N/A | N/A |
| rs7205040 | 0.87218 | 0.007483 |
| rs552219 | N/A | N/A |
| rs78045374 | 1.376345 | 0.006108 |
| rs117634357 | 1.8542 | 0.040457 |
| rs2236895 | 1.548361 | 0.043192 |
| rs7646021 | 1.623016 | 0.032633 |
| rs17152247 | 1.502065 | 0.017644 |
| rs10235518 | 0.748334 | 0.03971 |
| rs2296651 | N/A | N/A |

The lasso model was repeatedly fitted across 1000 iterations, using cross validation to find the optimal value for the penalty parameter. In case a coefficient was set to zero in all iterations, the covariate is excluded. The table below provides the mean and standard deviation of the estimated hazard ratios across the 1000 iterations.

Prediction of Clinical Relapse: The receiver operating characteristic (ROC) curve was analyzed for treatment regimen, EOT HBsAg (≥ 100 IU/mL vs < 100 IU/mL), rs4668818, rs948006, rs2934456, rs77586835, rs75876539, rs8050261, rs1542951, rs231770, rs9277535, or a combination of these covariates, in predicting clinical relapse (CR) after stop of treatment. The legend shows the ROC area under the curve (AUC) statistic for model comparison are represented in FIG. 19 (6 months), FIG. 20 (1 year) and FIG. 21 (2 years).

Table 12 shows the logistic regression model fit predicting clinical relapse before 6 months (odds ratio and 95% confidence interval), including treatment regimen, EOT HBsAg, and all SNPs predicting CR. These results can be used to estimate the probability to relapse before 6 months.

TABLE 12

| | | | |
|---|---|---|---|
| Coefficient | Estimate | 2.5% | 97.5% |
| (Intercept) | 0.0341478 | 0.0049045 | 0.1687935 |
| LAST_PR_CMTRTTENOFOVIR | 4.0082121 | 1.6551774 | 10.1058830 |
| LAST_PR_CMTRTOTHER | 2.7265879 | 0.5621263 | 12.2078710 |
| HBSAG_CATHIGH | 4.5521994 | 1.2928425 | 20.4945071 |
| rs4668818 | 5.2686986 | 1.3033335 | 23.4399717 |
| rs948006 | 2.8309109 | 0.9080412 | 8.9390690 |
| rs2934456 | 6.2267221 | 1.8787568 | 23.0140426 |
| rs77586835 | 1.4172358 | 0.6172963 | 3.2522801 |
| rs75876539 | 3.6722923 | 1.6319966 | 8.8360456 |
| rs8050261 | 3.0083553 | 1.3306571 | 7.1618779 |
| rs1542951 | 0.5653950 | 0.2874760 | 1.0765134 |
| rs231770 | 0.7902143 | 0.4224501 | 1.4444717 |
| rs9277535 | 0.8332430 | 0.4071541 | 1.6731941 |

Prediction of Virological Relapse: The receiver operating characteristic (ROC) curve was analyzed for treatment regimen, EOT HBsAg (≥ 100 IU/mL vs < 100 IU/mL), baseline HBeAg status, all 30 SNPs associated to virological relapse (VR), or a combination of these covariates, in predicting virological relapse (VR) after stop of treatment are represented on FIG. 22 (3 months), FIG. 23 (6 months), FIG. 24 (1 year) and FIG. 25 (2 years). The legend shows the ROC area under the curve (AUC) statistic for model comparison.

Table 13 shows the logistic regression model fit predicting viral relapse before 3 months (odds ratio and 95% confidence interval), including treatment regimen, EOT HBsAg, baseline HBeAg status, and all SNPs predicting VR. These results can be used to estimate the probability to relapse before 3 months.

TABLE 13

| | | | |
|---|---|---|---|
| Coefficient | Estimate | 2.5 % | 97.5 % |
| (Intercept) | 0.0000005 | 0.0000000 | 5.876900e-03 |
| LAST_PR_CMTRTTENOFOVIR | 3010.2828139 | 42.9974605 | 9.422707e+06 |
| LAST_PR_CMTRTOTHER | 315.5308045 | 1.5376558 | 1.329776e+06 |
| HBSAG_CATHIGH | 0.0984925 | 0.0001344 | 1.397314e+01 |
| HBAGSTATPRIOR TO / AT START OF NA TREATMENT (CURRENT THERAPY) | 0.1284966 | 0.0008333 | 9.565348e+00 |
| rs7534054 | 0.6446678 | 0.0011790 | 4.966513e+01 |
| rs4315565 | 1.0075868 | 0.0969034 | 1.263287e+01 |
| rs12105972 | 0.4563093 | 0.0652806 | 2.979234e+00 |
| rs1994245 | 2.6444688 | 0.3741923 | 3.232525e+01 |
| rs11836590 | 4.1520028 | 0.4870384 | 6.436303e+01 |
| rs7629161 | 4.1926387 | 0.3753428 | 8.360577e+01 |
| rs9828024 | 0.3329907 | 0.0213554 | 2.429800e+00 |
| rs7670984 | 74.0595850 | 6.0852191 | 4.434952e+03 |
| rs12645094 | 56.6487151 | 3.7892746 | 5.730291e+03 |
| rs2163787 | 22.7666174 | 0.8661059 | 3.072464e+03 |
| rs924446 | 79.6233236 | 3.3724650 | 2.000316e+04 |
| rs180001 | 7.6688344 | 0.8265262 | 2.240962e+02 |
| rs12199613 | 15.7687404 | 1.2678722 | 1.859552e+03 |
| rs2394952 | 1.9029435 | 0.1.330870 | 3.095553e+01 |
| rs17152258 | 2.5932388 | 0.2394907 | 4.569751e+01 |
| rs7459445 | 2.0498210 | 0.1653976 | 2.998906e+01 |
| rs1053403 | 0.8587095 | 0.0552701 | 9.284216e+00 |
| rs2767035 | 0.0135833 | 0.0002463 | 2.042183e-01 |
| rs3943102 | 0.3709716 | 0.0361703 | 2.125766e+00 |
| rs2154237 | 1.9301497 | 0.0748074 | 5.837891e+01 |
| rs73371840 | 2.3106896 | 0.3373420 | 2.919098e+01 |
| rs7205040 | 0.1516261 | 0.0100634 | 1.198150e+00 |
| rs552219 | 0.0861449 | 0.0008990 | 3.037723e+00 |
| rs78045374 | 16.4902243 | 1.1243242 | 8.778332e+02 |
| rs117634357 | 89.6765771 | 1.8793392 | 7.970978e+04 |
| rs2236895 | 194.7071540 | 2.1059475 | 2.946394e+05 |
| rs7646021 | 127.2214434 | 3.0996745 | 4.079425e+04 |
| rs17152247 | 110.2567452 | 4.4551262 | 2.279525e+04 |
| rs10235518 | 3.5529011 | 0.0748145 | 3.200812e+02 |
| rs2296651 | 0.1556924 | 0.0003594 | 3.957616e+01 |

Discussion: Several genetic markers have been already described in the literature (most often from targeted studies) as associated with persistent HBV infection, treatment response and disease progression (O′Brien TR, Yang HI, Groover S, Jeng W, “Spontaneous Clearance of Hepatitis C or B Virus, Response to Treatment, and Disease Progression,” J. Gastroenterology, 2019 Jan;156(2):400-417). In our study, we could replicate the observation of Su et al. (Su TH, Yang HC, Tseng TC, et al. “Distinct relapse rates and risk predictors after discontinuing tenofovir and entecavir therapy,” J. Infect. Dis. 2018; 217:1193-1201) who identified CTLA4 as a marker associated with clinical relapse on a NUC treatment discontinuation study including 100 Chronic HBV infected patients, confirming at large scale the central role of CTLA4 immune checkpoint (rs231770) onset of clinical relapse. A punctual mutation on HLA-DPB1 (rs9277535) has already been described as associated with HBV persistence (Thomas R, Thio CL, Apps R, et al. “A novel variant marking HLA-DP expression levels predicts recovery from hepatitis B virus infection,” J. Virol. 2012; 86: 6979-6985; Koukoulioti E, Fischer J, Schott E, Fülöp B, Heyne R, Berg T, van Bömmel F. “Association of HLA-DPA1 and HLA-DPB1 polymorphisms with spontaneous HBsAg seroclearance in Caucasians,” Liver Int. 2019 Apr; 39(4):646-654), but Su et al. could not identify a significant association with clinical relapse. In our large study, the same SNP predicts the onset of clinical relapse. The role of this specific mutation is HLA-DPB1 is not clear in HBV chronic infection. In a complementary screening approach as shown in the above Example 1, seven other SNPs showed significant association with time to Clinical Relapse, including on RBFOX1 (rs8050261) which was already described as a hotspot for HBV integration (Ding D, Lou X, Hua D, Yu W, Li L, Wang J, Gao F, Zhao N, Ren G, Li L, Lin B, “Recurrent targeted genes of hepatitis B virus in the liver cancer genomes identified by a next-generation sequencing-based approach,” PLoS Genet. 2012; 8(12):e1003065). Thirty three independent SNPs identified from a genome wide approach are predictive for onset of viral relapse, including a SNP (rs2154237) in FUT8 gene involved in glycosylation of NTCP receptor, independently of the NUC treatment. Interestingly, in the same cohort a mutation in SLC10A1 (presence of A allele in rs2296651), already known as having functional consequences on NTCP HBV entry receptor is protective of early viral relapse. The same mutation has already been described as protective for HBV persistence (Peng L1, Zhao Q, Li Q, Li M, Li C, Xu T, Jing X, Zhu X, Wang Y, Li F, Liu R, Zhong C, Pan Q, Zeng B, Liao Q, Hu B, Hu ZX, Huang YS, Sham P, Liu J, Xu S, Wang J, Gao ZL, Wang Y. “The p.Ser267Phe variant in SLC10A1 is associated with resistance to chronic hepatitis B,” Hepatology. 2015 Apr; 61(4):1251-60. doi: 10.1002/hep.27608. Epub 2015 Feb 23.) and progression (Zeng Z, Winkler CA. “The Loss-of-Function S267F Variant in HBV Receptor NTCP Reduces Human Risk for HBV Infection and Disease Progression. An P1,” J. Infect. Dis. 2018 Sep 22; 218(9):1404-1410), but to our knowledge it has never described as associated with viral relapse. With the same approach, several SNPs in the same linkage disequilibrium block in the HLA-C region (rs2394952) showed a strong association with onset of viral relapse. If its role in chronic HBV is still unknown, the same region has been described as associated with HBV clearance (Su et al.). Most of those markers are independent predictors for onset of relapse, improving both sensitivity and specificity in detecting viral relapse at 3 months and clinical relapse at 6 months after treatment discontinuation compared to clinical markers (HBsAg at the end of treatment and last NUC treatment for clinical relapse, and baseline HBeAg status for viral relapse). Host genetic markers are important contributors in predicting patient outcome.

### Example 2. Identification and Evaluation of Virological and Host Genetic Markers Associated with Sustained Clinical Response

The objective of this study was to evaluate virological and host genetic markers that can be associated with sustained clinical response (SCR) in chronic hepatitis B (CHB) patients following discontinuation of direct antiviral treatment.

### Materials and Methods

Patients and Study Design, Serology, and Genetic Data: They are the same as those in Example 1.

Statistical Analysis: Continuous variables are presented as means ± standard deviations, categorical variables by counts and percentages (see Table 14 below).

TABLE 14

Clinical characteristics of the study population

| Covariate | |
|---|---|
| Age (years) | 50.8 ± 11.3 |
| Gender | |
| Male | 142 (76.3%) |
| Female | 44 (23.7%) |
| Previous NA therapy | |
| Yes | 64 (34.4%) |
| No | 122 (65.6%) |
| Baseline HBeAg status | |
| Negativity prior to start of NA therapy | 142 (76.3%) |
| Negativity obtained during NA therapy | 44 (23.7%) |
| Treatment regimen | |
| Entecavir | 106 (57.0%) |
| Tenofovir | 65 (34.9%) |
| Other (Lamivudine, Telbivudine, Adefovir) | 15 (8.1%) |
| Length NA therapy (years) | 4.3 ± 1.9 |
| On treatment viral suppression | |
| No (HBV DNA all detected) | 26 (14.0%) |
| Consistent (HBV DNA all not detected) | 38 (20.4%) |
| Inconsistent (HBV DNA both detected and not detected) | 122 (65.6%) |
| End of treatment HBV DNA | |
| Detected | 96 (51.6%) |
| Not detected | 90 (48.4%) |
| End of treatment HBsAg | |
| < 100 IU/mL | 39 (21.0%) |
| ≥ 100 IU/mL | 147 (79.0%) |
| End of treatment HBsAg (log IU/mL) | 5.6 ± 2.3 |
| End of treatment HBsAb (log mIU/mL) | 0.16 ± 0.73 |
| End of treatment ALT (log U/L) | 3.1 ± 0.47 |
| End of treatment AST (log U/L) | 3.1 ± 0.29 |
| 1 month follow-up HBV DNA (log IU/mL) | 5.5 ± 5.0 |
| 1 month follow-up HBsAg (log IU/mL) | 5.5 ± 2.6 |
| 1 month follow-up ALT (log U/L) | 3.3 ± 0.79 |

To assess associations to sustained clinical response, logistic regression analysis was performed. All statistical analyses were executed within the R (3.6.1) statistical software. Statistical tests were two-sided and considered significant at the 0.05 significance level (see Table 15 below).

Statistical Analysis of Genetics Data: All the statistical analyses were performed in R (3.4.3). Considering that HBsAg status was representative of disease progression, the association between the SNPs and sustained clinical response was assessed using a logistic model, considering HBsAg levels (high (≥ 100 IU/mL) vs. low (< 100 IU/mL)) at the last visit before end of antiviral treatment.

In these models the GENETIC term corresponds to the SNP encoded using the additive (AA=0, AB=1, BB=2), dominant (AA=0, AB=1, BB=1) or recessive (AA=0, AB=0, BB=1) genetic models.

The association with sustained clinical response (SCR) models were run univariately and independently for every SNP and each of the three genetic models.

The analysis included on the one hand 29 SNPs previously identified from a genetic scan as significantly associated with onset of viral relapse (see Table 9 in Example 1) and on the other hand 22 SNPs (see Table 1 in Example 1) described in previous studies as associated with Chronic hepatitis B. Table 16 shows the overview of the total of 51 candidate SNPs (29 SNPs identified as protective for early onset of viral relapse with an FDR<5% and 22 identified from the literature). For each rsid, the closest gene (in chromosomal location) is indicated together with the origin of the SNP (reason for selection as candidate), the corresponding minor and major alleles at this locus, and the Major Allele Frequency.

TABLE 16

| RSID | GENE | ORIGIN | MINOR ALLELE | MAJOR ALLELE | MAF |
|---|---|---|---|---|---|
| rs2296651_AX_12524927 | SLC10A1 | CANDIDATE_SCAN | A | G | 0.05769 |
| rs3130542_AX_11435438 | HLA-C | CANDIDATE_SCAN | A | G | 0.2225 |
| rs1419881_AX_41950487 | TCF19 | CANDIDATE_SCAN | G | A | 0.4478 |
| rs7574865_AX_11630908 | STAT4 | CANDIDATE_SCAN | T | G | 0.3343 |
| rs2291617_AX_11382422 | METTL21B | CANDIDATE_SCAN | T | G | 0.2912 |
| rs9692372_AX_15654248 | --- | CANDIDATE_SCAN | C | A | 0.3929 |
| rs2000478_AX_16645527 | --- | CANDIDATE_SCAN | T | G | 0.217 |
| rs3077_AX_11432694 | HLA-DPA1 | CANDIDATE_SCAN | A | G | 0.1923 |
| rs9272105_AX_165873764 | HLA-DQA1 | CANDIDATE_SCAN | G | A | 0.4203 |
| rs9272105_AX_41957017 | HLA-DQA1 | CANDIDATE_SCAN | G | A | 0.418 |
| rs169083_AX_15121359 | --- | CANDIDATE_SCAN | T | C | 0.2088 |
| rs9277535_AX_11677618 | HLA-DPB1 | CANDIDATE_SCAN | A | G | 0.2418 |
| rs12979860_AX_40445375 | IFNL4 | CANDIDATE_SCAN | T | C | 0.04645 |
| rs12979860_AX_112063628 | IFNL4 | CANDIDATE_SCAN | T | C | 0.0467 |
| rs8099917_AX_11665728 | --- | CANDIDATE_SCAN | G | T | 0.0467 |
| rs2856718_AX_11419599 | HLA-DQB1 | CANDIDATE_SCAN | C | T | 0.4588 |
| rs455804_AX_11511204 | GRIK1 | CANDIDATE_SCAN | A | C | 0.3626 |
| rs231770_AX_11384541 | CTLA4 | CANDIDATE_SCAN | C | T | 0.3379 |
| rs4821116_AX_12582596 | UBE2L3 | CANDIDATE_SCAN | T | C | 0.3978 |
| rs652888_AX_41954239 | EHMT2 | CANDIDATE_SCAN | G | A | 0.3077 |
| rs7453920_AX_11625220 | HLA-DQB2 | CANDIDATE_SCAN | A | G | 0.03757 |
| rs12356193_AX_11202033 | SLC16A9 | CANDIDATE_SCAN | G | A | 0.002747 |
| rs7534054_AX_38931131 | CASZ1 | VIRAL_GWAS | A | C | 0.1236 |
| rs180001_AX_11344578 | ATXN1 | VIRAL_GWAS | G | A | 0.3564 |
| rs4315565_AX_92330226 | ANTXR1 | VIRAL_GWAS | A | G | 0.3114 |
| rs2154237_AX_12519032 | FUT8 | VIRAL_GWAS | G | T | 0.1077 |
| rs10235518_AX_12386027 | LSM8 | VIRAL_GWAS | C | T | 0.3333 |
| rs9828024_AX_41069901 | --- | VIRAL_GWAS | G | A | 0.4503 |
| rs924446_AX_41673685 | --- | VIRAL_GWAS | T | C | 0.3874 |
| rs12105972_AX_11190505 | --- | VIRAL_GWAS | C | G | 0.4011 |
| rs2767035_AX_39054979 | PDHX | VIRAL_GWAS | C | T | 0.3626 |
| rs7205040_AX_12624563 | PRKCB | VIRAL_GWAS | T | C | 0.3022 |
| rs3943102_AX_12565142 | --- | VIRAL_GWAS | T | C | 0.3934 |
| rs12645094_AX_34633581 | NPY2R | VIRAL_GWAS | C | A | 0.4724 |
| rs73371840_AX_31286185 | C14orf80 | VIRAL_GWAS | C | T | 0.3791 |
| rs7629161_AX_41281397 | LINC00877 | VIRAL_GWAS | T | C | 0.2444 |
| rs1053403_AX_11116332 | SEC61A2 | VIRAL_GWAS | G | A | 0.4725 |
| rs552219_AX_88894037 | LAMA1 | VIRAL_GWAS | A | G | 0.2167 |
| rs2394952_AX_11389327 | HLA-C | VIRAL_GWAS | G | A | 0.2225 |
| rs11896590_AX_12422368 | TMEFF2 | VIRAL_GWAS | G | A | 0.4423 |
| rs17152258_AX_42075557 | CDHR3 | VIRAL_GWAS | T | C | 0.2088 |
| rs1994245_AX_11358423 | TMEFF2 | VIRAL_GWAS | C | T | 0.2983 |
| rs12199613_AX_15347401 | BTN3A2 | VIRAL_GWAS | T | C | 0.2418 |
| rs7459445_AX_11625332 | --- | VIRAL_GWAS | C | T | 0.1346 |
| rs17152247_AX_15510825 | CDHR3 | VIRAL_GWAS | T | C | 0.4836 |
| rs2163787_AX_14924228 | SCGB3A2 | VIRAL_GWAS | A | G | 0.1236 |
| rs117634357_AX_31132439 | TP73 | VIRAL_GWAS | T | C | 0.1831 |
| rs2236895_AX_11377303 | LAMB3 | VIRAL_GWAS | T | G | 0.1721 |
| rs7646021_AX_14341238 | CADPS | VIRAL_GWAS | G | T | 0.4481 |
| rs78045374_AX_32826619 | --- | VIRAL_GWAS | C | T | 0.1126 |
| rs7670984_AX_14795137 | GRID2 | VIRAL_GWAS | A | C | 0.1071 |

Due to the relative small number of patients experiencing sustained clinical response (23 out of 186), the full genomic scan could not be performed comparing the genetic profile of patients experiencing sustained clinical response with the genetic profile of patients experiencing either a viral relapse or a clinical relapse during the first two years following the end of their NUC treatment.

### Results

Study Population: Table 14 provides an overview of the clinical characteristics of the study population. In total, 101 patients completed the study, for 83 patients nucleos(t)ide analog (NA) therapy was re-initiated due to relapse. One patient died, and one patient was diagnosed with hepatocellular carcinoma during the study period. The follow-up time after stop of treatment ranges from 38 to 814 days, with a mean and median period of follow-up of 482 and 637 days, respectively.

Out of the 186 patients included in the cohort, 161 (86.6%) experienced a virological relapse, of whom 110 (59.1%) also had a clinical relapse. For 23 patients (12.4%), sustained clinical response was observed over the follow-up period (note that one patient who died and one patient diagnosed with hepatocellular carcinoma were excluded from the sustained clinical response group). 51 (27.4%) patients had a virological relapse without a clinical relapse. In the entire follow up period, 11 patients lost HBsAg. Out of those 11 HBsAg loss patients, 7 were also considered as sustained clinical responders. Three experienced a virological relapse before losing HBsAg, and the fourth being the patient diagnosed with hepatocellular carcinoma and not considered a sustained clinical responder.

Sustained Clinical Response: Logistic regression shows the negative association of prior HBeAg negativity at the start of NA therapy (OR, 0.25; 95% CI, 0.06-0.99; P = 0.05), high HBsAg at the end of treatment (OR, 0.16; 95% CI, 0.04-0.55; P = 0.005), and higher HBV DNA 1 month after stop treatment (OR, 0.54; 95% CI, 0.28-0.78; P = 0.01), to sustained clinical response (Table 15).

TABLE 15

Logistic regression analysis for sustained clinical response

| Covariate | Univariate analysis | | Multivariate analysis | |
|---|---|---|---|---|
| | OR (95% CI) | P value | OR (95% CI) | P value |
| Age (years) | 0.98 (0.94-1.01) | 0.21 | | |
| Gender (male vs female) | 0.43 (0.17-1.10) | 0.07 | | |
| Previous NA therapy (yes vs no) | 0.64 (0.22-1.63) | 0.37 | | |
| Baseline HBeAg status (prior negativity vs during treatment) | 0.34 (0.14-0.87) | 0.02 | 0.25 (0.06-0.99) | 0.048 |
| Tenofovir vs entecavir | 0.79 (0.29-2.03) | 0.64 | | |
| Other vs entecavir | 1.01 (0.15-4.20) | 0.99 | | |
| Length NA therapy (years) | 1.01 (0.79-1.24) | 0.93 | | |
| Inconsistent vs consistent | 0.81 (0.30-2.40) | 0.68 | | |
| No vs consistent | 0.21 (0.01-1.36) | 0.16 | | |
| HBV DNA at end of treatment (detected vs not detected) | 0.69 (0.28-1.65) | 0.41 | | |
| HBsAg at end of treatment (≥ 100 IU/mL vs < 100 IU/mL) | 0.23 (0.09-0.57) | 0.001 | 0.16 (0.04-0.55) | 0.005 |
| HBsAb at end of treatment (log mIU/mL) | 1.32 (0.78-2.04) | 0.22 | | |
| ALT at end of treatment (log U/L) | 1.40 (0.55-3.59) | 0.48 | | |
| AST at end of treatment (log U/L) | 1.34 (0.29-5.79) | 0.7 | | |
| HBV DNA at 1 month after stop treatment (log IU/mL) | 0.53 (0.27-0.77) | 0.01 | 0.54 (0.28-0.78) | 0.01 |
| ALT at 1 month after stop treatment (log U/L) | 0.59 (0.25-1.21) | 0.2 | | |
| HBsAg at 1 month after stop treatment (log IU/mL) | 0.72 (0.59-0.86) | <0.001 | | |

The mean and median HBsAg at the end of treatment among the 23 sustained clinical responders is 1854 IU/mL and 195 IU/mL, respectively, with values ranging from 0.05 IU/mL to 32630 IU/mL.

The majority (22 out of 29) of SNPs previously reported as associated with the onset of virological relapse, showed an association with sustained clinical response (SCR) (Table 17, FIG. 28). Table 17 provides an overview of all 51 SNPs tested for association with sustained clinical response. Among them, 24 SNPs show a significant association with SCR on a logistic model, adjusting for HBsAg levels at the end of the treatment (p-value<0.05). The SNPs are ordered by significance (p-value) and Area Under the Curve, derived from Receiver Operating Characteristic curve analysis. MAF=Minor Allele Frequency. AUC= Area Under the curve. FIG. 28 shows barplot of the distribution of the 24 candidate SNPS, indicating a significant association with sustained clinical response for the 3 models tested (additive = ADD, dominant = DOM, recessive = REC) ordered by significance. The rsid is indicated for each SNP, together with its corresponding probe set ID (ThermoFisher UK Biobank or Asia PMRA). In black is represented the population of patients who experienced either a viral relapse or a clinical relapse during the 2 years follow up period. In grey is represented the population of patients who experienced sustained clinical response.

TABLE 17

| | | | | | | | | | | | | | | | |
|---|---|---|---|---|---|---|---|---|---|---|---|---|---|---|---|
| RSID | GENE | ORIGIN | MINOR ALLELE | MAJOR ALLELE | MAP | 0 | 1 | 2 | ALLELES | Model | estimate | p.value | AUC | ALLELE PROTECTIVE | ALLELE RISK |
| rs7534054 | CASZ1 | VIRAL_GWAS | A | C | 0.1236 | CC | AC | AA | CC (0) - AC (1) - AA (2) | ADD | 2.009565 | 1.98E-05 | 0.7808 | A | C |
| rs180001 | ATXN1 | VIRAL_GWAS | G | A | 0.3564 | AA | GA | GG | AA (0) - GA (1) - GG (2) | ADD | 1.544927 | 9.91E-05 | 0.787 | G | A |
| rs4315565 | ANTXR1 | VIRAL_GWAS | A | G | 0.3114 | GG | AG | AA | GG (0) - AG (1) - AA (2) | ADD | 1.509224 | 0.000155 | 0.8186 | A | G |
| rs2154237 | FUT8 | VIRAL_GWAS | G | T | 0.1077 | TT | GT | GG | TT (0) - GT (1) -GG (2) | ADD | 1.874174 | 0.000155 | 0.7581 | G | T |
| rs10235518 | LSM8 | VIRAL_GWAS | C | T | 0.3333 | TT | CT | CC | TT (0) - CT (1) - CC (2) | REC | 2.052922 | 0.000223 | 0.7405 | CC | T |
| rs9828024 | | VIRAL_GWAS | G | A | 0.4503 | AA | GA | GG | AA (0) - GA (1) - GG (2) | ADD | 1.412379 | 0.000239 | 0.7749 | G | A |
| rs924446 | | VIRAL_GWAS | T | C | 0.3874 | CC | TC | TT | CC (0) - TC (1) - TT (2) | ADD | -1.87341 | 0.000282 | 0.8033 | C | T |
| rs12105972 | | VIRAL_GWAS | C | G | 0.4011 | GG | CG | CC | GG (0) - CG (1) -CC (2) | ADD | -1.6977 | 0.000379 | 0.7964 | G | C |
| rs2767035 | PDHX | VIRAL_GWAS | C | T | 0.3626 | TT | CT | CC | TT (0) - CT (1) - CC (2) | ADD | 1.323577 | 0.000453 | 0.7815 | C | T |
| rs7205040 | PRKCB | VIRAL_GWAS | T | C | 0.3022 | CC | TC | TT | CC (0) - TC (1) -TT (2) | ADD | 1.196834 | 0.000721 | 0.7711 | T | C |
| rs3943102 | --- | VIRAL_GWAS | T | C | 0.3934 | CC | TC | TT | CC (0) - TC (1) - TT (2) | ADD | -1.49203 | 0.000948 | 0.7764 | C | T |
| rs12645094 | NPY2R | VIRAL_GWAS | C | A | 0.4724 | AA | CA | CC | AA (0) - CA (1) - CC (2) | ADD | -1.34657 | 0.001003 | 0.786 | A | C |
| rs73371840 | C14orf80 | VIRAL_GWAS | C | T | 0.3791 | TT | CT | CC | TT (0) - CT (1) - CC (2) | ADD | 1.061706 | 0.002803 | 0.7614 | C | T |
| rs7629161 | LINC00877 | VIRAL_GWAS | T | C | 0.2444 | CC | TC | TT | CC (0) - TC (1) - TT (2) | ADD | 1.057212 | 0.003809 | 0.7672 | T | C |
| rs1053403 | SEC61A2 | VIRAL_GWAS | G | A | 0.4725 | AA | GA | GG | AA (0) - GA (1) - GG (2) | ADD | 1.010636 | 0.00456 | 0.7573 | G | A |
| rs552219 | LAMA1 | VIRAL_GWAS | A | G | 0.2167 | GG | AG | AA | GG (0) - AG (1) - AA (2) | ADD | 1.063744 | 0.005655 | 0.7407 | A | G |
| rs2296651 | SLC10A1 | CANDIDATE SCAN | A | G | 0.05769 | GG | AG | #N/A | GG (0) - AG (1) | ADD | 1.659699 | 0.005366 | 0.7092 | A | G |
| rs3130542 | HLA-C | CANDIDATE SCAN | A | G | 0.2225 | GG | AG | AA | GG (0) - AG (1) - AA (2) | ADD | -1.73127 | 0.006898 | 0.7562 | G | A |
| rs2394952 | HLA-C | VIRAL_GWAS | G | A | 0.2225 | AA | GA | GG | AA (0) - GA (1) - GG (2) | ADD | -1.73127 | 0.006898 | 0.7562 | A | G |
| ..11896590 | TMEFF2 | VIRAL_GWAS | G | A | 0.4423 | AA | GA | GG | AA (0) - GA (1) - GG (2) | ADD | 0.949445 | 0.008185 | 0.7291 | G | A |
| rs17152258 | CDHR3 | VIRAL_GWAS | T | C | 0.2088 | CC | TC | TT | CC (0) - TC (1) - TT (2) | ADD | -1.94637 | 0.009913 | 0.7475 | C | T |
| rs1994245 | TMEFF2 | VIRAL_GWAS | C | T | 0.2983 | TT | CT | CC | TT (0) - CT (1) - CC (2) | ADD | -1.12437 | 0.01493 | 0.7426 | T | C |
| rs12199613 | BTN3A2 | VIRAL_GWAS | T | C | 0.2418 | CC | TC | TT | CC (0) - TC (1) - TT (2) | ADD | -1.30741 | 0.019419 | 0.7225 | C | T |
| rs7459445 | | VIRAL_GWAS | C | T | 0.1346 | TT | CT | #N/A | TT (0) - CT (1) | ADD | -2.15637 | 0.038937 | 0.7235 | T | C |
| rs17152247 | CDHR3 | VIRAL_GWAS | T | C | 0.4836 | CC | TC | TT | CC (0) - TC (1) - TT (2) | ADD | -0.64253 | 0.065067 | 0.7186 | C | T |
| rs2163787 | SCGB3A2 | VIRAL_GWAS | A | G | 0.1236 | GG | AG | AA | GG (0) - AG (1) - AA (2) | ADD | -1.20315 | 0.099624 | 0.687 | G | A |
| rs117634357 | TP73 | VIRAL_GWAS | T | C | 0.1831 | CC | TC | TT | CC (0) - TC (1) - TT (2) | ADD | -0.57564 | 0.162374 | 0.6639 | C | T |
| rs2236895 | LAMB3 | VIRAL_GWAS | T | G | 0.1721 | GG | TG | TT | GG (0) - TG (1) - TT (2) | DOM | 0.678042 | 0.164054 | 0.6894 | T | GG |
| rs1419881 | TCF19 | CANDIDATE SCAN | G | A | 0.4478 | AA | GA | GG | AA (0) - GA (1) - GG (2) | ADD | -0.42715 | 0.192559 | 0.6682 | A | G |
| rs7574865 | STAT4 | CANDIDATE SCAN | T | G | 0.3343 | GG | TG | TT | GG (0) - TG (1) - TT (2) | DOM | 0.624042 | 0.19509 | 0.6919 | T | GG |
| rs2291617 | METTL21B | CANDIDATE SCAN | T | G | 0.2912 | GG | TG | TT | GG (0) - TG (1) - TT (2) | ADD | 0.44391 | 0.197293 | 0.69 | T | G |
| rs9692372 | | CANDIDATE SCAN | C | A | 0.3929 | AA | CA | CC | AA (0) - CA (1) - CC (2) | REC | -1.36051 | 0.205738 | 0.681 | A | CC |
| rs2000478 | | CANDIDATE SCAN | T | G | 0.217 | GG | TG | TT | GG (0) - TG (1) -TT (2) | ADD | 0.44635 | 0.225525 | 0.674 | T | G |
| rs3077 | HLA-DPA1 | CANDIDATE SCAN | A | G | 0.1923 | GG | AG | AA | GG (0) - AG (1) - AA (2) | DOM | 0.573446 | 0.226811 | 0.7058 | A | GG |
| rs7646021 | CADPS | VIRAL_GWAS | G | T | 0.4481 | TT | GT | GG | TT (0) - GT (1) -GG (2) | DOM | 0.696726 | 0.237261 | 0.7064 | G | TT |
| rs9272105 | HLA-DQA1 | CANDIDATE SCAN | G | A | 0.4203 | AA | GA | GG | AA (0) - GA (1) - GG (2) | REC | -0.72499 | 0.284249 | 0.6786 | A | GG |
| rs9272105 | HLA-DQA1 | CANDIDATE SCAN | G | A | 0.418 | AA | GA | GG | AA (0) - GA (1) - GG (2) | REC | -0.7209 | 0.287086 | 0.6788 | A | GG |
| rs169083 | --- | CANDIDATE SCAN | T | C | 0.2088 | CC | TC | TT | CC (0) - TC (1) - TT (2) | ADD | -0.42304 | 0.337094 | 0.6738 | C | T |
| rs9277535 | HLA-DPB1 | CANDIDATE SCAN | A | G | 0.2418 | GG | AG | AA | GG (0) - AG (1) - AA (2) | DOM | 0.436792 | 0.346139 | 0.7034 | A | GG |
| rs12979860 | IFNL4 | CANDIDATE SCAN | T | C | 0.04645 | CC | TC | TT | CC (0) - TC (1) - TT (2) | DOM | 0.614325 | 0.38901 | 0.6736 | T | CC |
| rs12979860 | IFNL4 | CANDIDATE SCAN | T | C | 0.0467 | CC | TC | TT | CC (0) - TC (1) - Tr (2) | DOM | 0.608847 | 0.393096 | 0.673 | T | CC |
| rs8099917 | --- | CANDIDATE SCAN | G | T | 0.0467 | TT | GT | GG | TT (0) - GT (1) -GG (2) | DOM | 0.608847 | 0.393096 | 0.673 | G | TT |
| rs2856718 | HLA-DQB1 | CANDIDATE SCAN | C | T | 0.4588 | TT | CT | CC | TT (0) - CT (1) - CC (2) | ADD | 0.224493 | 0.440205 | 0.6637 | C | T |
| rs455804 | GRIK1 | CANDIDATE SCAN | A | C | 0.3626 | CC | AC | AA | CC (0) - AC (1) - AA (2) | REC | 0.465428 | 0.454863 | 0.6635 | AA | C |
| rs231770 | CTLA4 | CANDIDATE SCAN | C | T | 0.3379 | TT | CT | CC | TT (0) - CT (1) - CC (2) | DOM | -0.32951 | 0.4779 | 0.6646 | TT | C |
| rs4821116 | UBE2L3 | CANDIDATE SCAN | T | C | 0.3978 | CC | TC | TT | CC (0) - TC (1) - TT (2) | REC | 0.392734 | 0.497951 | 0.6652 | TT | C |
| rs652888 | EHMT2 | CANDIDATE SCAN | G | A | 0.3077 | AA | GA | GG | AA (0) - GA (1) - GG (2) | REC | -0.60939 | 0.579301 | 0.6538 | A | GG |
| rs7453920 | HLA-DQB2 | CANDIDATE SCAN | A | G | 0.03757 | GG | AG | AA | GG (0) - AG (1) - AA (2) | ADD | -0.32728 | 0.751006 | 0.6643 | G | A |
| rs78045374 | --- | VIRAL GWAS | C | T | 0.1126 | TT | CT | CC | TT (0) - CT (1) - CC (2) | REC | -14.7335 | 0.989737 | 0.6639 | T | CC |
| rs7670984 | GRlD2 | VIRAL GWAS | A | C | 0.1071 | CC | AC | AA | CC (0) - AC (1) - AA (2) | ADD | -17.7552 | 0.991069 | 0.7384 | C | A |
| rs12356193 | SLC16A9 | CANDIDATE SCAN | G | A | 0.002747 | AA | GA | #N/A | AA (0) - GA (1) | ADD | -13.1758 | 0.992777 | 0.6559 | A | G |

Two candidate SNPs (rs3130542, in HLA-C, and rs2296651 in SLC10A1) showed a significant association with sustained clinical response (SCR). The AUC (Area Under the Curve) of the ROC (Receiver Operating Characteristic) curves predicting SCR including the newly identified markers, and in addition a main effect for HBsAg (high vs. low, as described above) vary between 0.71 and 0.82, compared to 0.65 when including HBsAg alone.

Interestingly, two of those markers, SNPs in FUT8 (rs2154237) and SLC10A1 (rs2296651), reported as having a functional effect on NTCP receptor, are associated with sustained clinical response. Different haplotypes were derived from the genotypes observed in FUT8 and SLC10A1, representing an additive and dominant model (Table 18 and Table 19).

TABLE 18

FUT8 (rs2154237) / SLC10A1 (rs2296651) haplotype using an additive model

| Combination | Additive model |
|---|---|
| FUT8: T/T (0) - SLC10A1: G/G (0) | 0 |
| FUT8: T/T (0) - SLC10A1: A/G (1) | 1 |
| FUT8: G/T (1) - SLC10A1: G/G (II) | 1 |
| FUT8: G/T (1) - SLC10A1: A/G (1) | 2 |
| FUT8: G/G (2) - SLC10A1: G/G (0) | 2 |

TABLE 19

FUT8 (rs2154237) / SLC10A1 (rs2296651) haplotype using a dominant model

| Combination | Dominant model |
|---|---|
| FUT8: T/T (0) - SLC10A1: G/G (0) | 0 |
| FUT8: T/T (0) - SLC10A1: A/G (1) | 1 |
| FUT8: G/T (1) - SLC10A1: G/G (0) | 1 |
| FUT8: G/T (1) - SLC10A1: A/G (1) | 1 |
| FUT8: G/G (2) - SLC10A1: G/G (0) | 1 |

Both rs2296651 (SLC10A1) and rs2154237 (FUT8) are independent predictors of sustained clinical response improving sensitivity and specificity as shown in the ROC analysis (FIG. 29), with an AUC from 0.65 (HBsAg) to 0.80 (including the two genetic markers).

Based on the ROC analyses, a specific model with enough sensitivity and specificity could be selected to define a classification rule.

Given the high observed AUC, a logistic regression model could be considered including main effects for HBsAg, rs2154237 (FUT8), and rs2296651 (SLC10A1) genotype to predict sustained clinical response within follow-up after stop treatment (Table 20).

TABLE 20

| Term | Estimate | Standard error | Statistic | p.value |
|---|---|---|---|---|
| (Intercept) | -1398337 | 0.412861 | -3.3869 | 0.00071 |
| HBsAg (>100 IU/ml) | -2108174 | 0.579881 | -3.6355 | 0.00028 |
| FUT8 (rs2154237) | 1.869603 | 0.508069 | 3.67982 | 0.00023 |
| SLC10A1 (rs2296631) | 1.736152 | 0.663682 | 2.61594 | 0.0089 |

With a threshold of 18.05% on the predicted probability (i.e. in case of a predicted probability higher than the threshold, the prediction for response is true), the model has a sensitivity of 65.22% and a specificity of 81.53%. Table 21 shows the observed number of sustained clinical response in the rows and the predicted response based on the logistic regression model in the columns.

TABLE 21

| Observed/predicted SCR | FALSE | TRUE | Total |
|---|---|---|---|
| FALSE | 128 | 29 | 157 |
| TRUE | 8 | 15 | 23 |
| Total | 136 | 44 | 180 |

Using the model fit above, the predicted probability for sustained clinical response after stop treatment can be computed for all possible combinations of the covariates. Table 22 provides a summary (rule) for predicting SCR using those parameters (given the threshold of 18.05% on the predicted probability in the logistic model).

TABLE 22

| rs2154237 (FUT8) | rs2296651 (SLC10A1) | HBsAg | Prediction |
|---|---|---|---|
| 2 | 1 | x | SCR |
| 2 | 0 | x | SCR |
| 1 | 1 | x | SCR |
| 1 | 0 | Low | SCR |
| 1 | 0 | High | NO SCR |
| 0 | 1 | Low | SCR |
| 0 | 1 | High | NO SCR |
| 0 | 0 | Low | SCR |
| 0 | 0 | High | NO SCR |

However, for some combinations only very few observations are present in the data, therefore validation on an external dataset would definitely be important to assess the robustness of the model.

### Discussion

This observational study aimed to identify virological and host genetic predictive markers for sustained clinical response in chronic hepatitis B (CHB) patients discontinuing direct antiviral treatment. In a cohort of 186 patients, 23 CHB patients were stable over the two years following discontinuation of the NUC treatment (neither viral nor clinical relapse was observed during the follow up of the patients). Low HBsAg level (<100 IU/ml) at the end of treatment was positively associated with SCR in a multivariate analysis, together with the absence of HBeAg negativity at the start of treatment and low HBV DNA the first month after NUC discontinuation.

Low HBsAg and steep decline in HBV DNA have been described as accepted viral biomarkers predicting long term response and seroclearance of HBsAg (Mak, et al., Hepatology International, 2020, 14: 35-46; Seto, et al., The Lancet, 2018, 392: 2313-24; Park, et al., World Journal of Gastroenterology 2016, 22: 9836-43; Lim, et al., Hepatology International 2016, 10: 829-37; Mommeja-Marin, et al, Hepatology 2003, 37: 1309-19; Jeng, et al., Hepatology 2018, 68: 425-34).

In a large retrospective study, among subjects that eventually cleared HBsAg, a lag of more than 4 years between the first drop of HBV DNA and functional cure could be reported (Iloeje, et al., Liver International 2012, 32: 1333-41) highlighting the need to identify new predictive biomarkers that could be measured during treatment and predict long term treatment response.

Several genetic markers have been reported in independent studies as being associated with disease susceptibility, HBsAg seroclearance, treatment response and disease progression to hepatocellular carcinoma (HCC) (O′Brien, et al., Gastroenterology 2019, 156: 400-17).

Most of those studies focused on targeted genetic association, especially on single nucleotide polymorphisms located on the Human Leukocyte Antigen (HLA) class II region (Wang, et al., Journal of Immunology Research, 2016, DOI:10.1155/2016/9069375; Akcay, et al., World Journal of Gastroenterology 2018, 24: 3347-60). Two of the previously identified markers associated with HBV showed a significant association with SCR in our study, including one in the HLA-C region (rs3130542, corresponding to HLA-C*02 allele). This SNP has been reported in a large Han Chinese cohort as associated with susceptibility to infection (G allele being protective for infection). HLA-C encodes a type I HLA molecule. An increased HLA-C expression (G allele in rs3130542 being associated with higher expression of HLA-C) in hepatocytes could stimulates attack by cytotoxic T cells (CTLs), a key component of virus elimination in chronic HBV carrier (Hu, et al., Nature Genetics 2013, 45: 1499-503).

HLA-C molecules can interact with killer immunoglobin-like receptors (KIRs) on the surface of natural killer (NK) cells, known to be less abundant in CHB patients (Rehermann, et al., Gastroenterology 2019, 156: 369-83; Stelma, et al., Journal of Viral Hepatitis 2016, 23: 652-9). In our study, G allele is enriched in patients experiencing SCR, and might contribute to immune regulation.

To our knowledge, this is the first time that rs2296651 has been identified as a protective marker for SCR (A allele has shown to be protective of early onset of viral relapse in the same cohort), suggesting that even once a patient is chronically infected with HBV, the functionality of NTCP receptor and its affinity for hepatitis B virus has an impact on the ability for the virus to infect new hepatocytes and that p.Ser267Phe variant is protective for relapse.

The majority of SNPs identified as protective for early onset of viral relapse (22/29) were also associated with SCR, confirming the importance of host genetic profiling in controlling chronic infection. In this cohort rs2154237 was associated with the onset of viral relapse and with SCR. In a multivariate model, rs2296651 and rs2154237 were independent predictors of SCR, considering HBsAg level at the end of treatment as a main effect.

### Example 3. Identification and Evaluation of Virological and Host Genetic Markers Associated with Relapse after Stopping (NUC) Treatment

The objective of this study was to evaluate a host genetic signature that can be predictive for onset of relapse in chronic hepatitis B (CHB) patients following discontinuation of direct antiviral treatment, in addition to HBsAg level at the end of treatment and the treatment regimen.

### Materials and Methods

Patients and Study Design, Serology, and Genetic Data: They are the same as those in Example 1.

Statistical Analysis: Clinical characteristic of the analysis dataset are provided in Table 14 in Example 2. Continuous variables are presented as means ± standard deviations, categorical variables by counts and percentages.

Cumulative incidence of virological and clinical relapse after stop of treatment was assessed by Kaplan-Meier curves. In addition, Cox proportional hazard regression analysis (Therneau Terry M., et al., Statistics in Medicine. 2001, 20(13):2053-2054) was applied, obtaining univariate and multivariate models for time to virological and clinical relapse (model fits are provided in Table 23a and Table 23b for virological and clinical relapse respectively).

TABLE 23a

Logistic regression analysis for virological relapse

| Covariate | Univariate analysis | P value Univariate analysis | Multivariate analysis | P value Multivariate analysis |
|---|---|---|---|---|
| | HR (95% CI) | | HR (95% CI) | |
| Age (years) | 1.01 (1.00-1.03) | 0.05 | 1.02 (1.00-1.03) | 0.03 |
| Gender (male vs female) | 1.53 (1.05-2.23) | 0.03 | | |
| Previous NA therapy (yes vs no) | 1.64 (1.19-2.27) | 0.003 | | |
| Baseline HBeAg status (prior negativity vs during treatment) | 2.02 (1.37-2.98) | <0.001 | 2.20 (1.43-3.40) | <0.001 |
| Treatment regimen | | | | |
| Tenofovir vs entecavir | 2.49 (1.77-3.49) | <0.001 | 2.97 (2.10-4.20) | <0.001 |
| Other vs entecavir | 1.57 (0.88-2.81) | 0.13 | 1.61 (0.90-2.90) | 0.11 |
| Length NA therapy (years) | 0.99 (0.91-1.07) | 0.79 | | |
| On-treatment viral suppression | | | | |
| Inconsistent vs consistent | 0.94 (0.63-1.41) | 0.77 | | |
| No vs consistent | 1.17 (0.69-2.00) | 0.56 | | |
| HBV DNA at end of treatment (detected vs not detected) | 1.08 (0.79-1.47) | 0.64 | | |
| HBsAg at end of treatment (≥ 100 IU/mL vs < 100 IU/mL) | 1.64 (1.08-2.49) | 0.02 | 2.37 (1.51-3.74) | <0.001 |
| HBsAb at end of treatment (log mIU/mL) | 0.92 (0.73-1.16) | 0.49 | | |
| ALT at end of treatment (log U/L) | 1.09 (0.81-1.48) | 0.57 | | |
| AST at end of treatment (log U/L) | 1.03 (0.62-1.70) | 0.93 | | |
| HBV DNA at 1 month after stop treatment (log IU/mL) | 1.21 (1.16-1.26) | <0.001 | | |
| ALT at 1 month after stop treatment (log U/L) | 1.55 (1.23-1.97) | <0.001 | | |
| HBsAg at 1 month after stop treatment (log IU/mL) | 1.08 (1.01-1.16) | 0.03 | | |

TABLE 23b

Logistic regression analysis for clinical relapse

| Covariate | Univariate analysis | P value univariate analysis | Multivariate analysis | P value multivariate analysis |
|---|---|---|---|---|
| | HR (95% CI) | | HR (95% CI) | |
| Age (years) | 1.01 (0.99-1.02) | 0.43 | 1.02 (1.00-1.04) | 0.02 |
| Gender (male vs female) | 1.88 (1.15-3.09) | 0.01 | | |
| Previous NA therapy (yes vs no) | 1.26 (0.86-1.86) | 0.24 | | |
| Baseline HBeAg status (prior negativity vs during treatment) | 1.35 (0.85-2.14) | 0.2 | | |
| Treatment regimen | | | | |
| Tenofovir vs entecavir | 1.77 (1.19-2.62) | 0.005 | 1.87 (1.26-2.78) | 0.002 |
| Other vs entecavir | 1.07 (0.51-2.25) | 0.85 | 1.21 (0.58-2.54) | 0.61 |
| Length NA therapy (years) | 0.96 (0.86-1.06) | 0.39 | | |
| On-treatment viral suppression | | | | |
| Inconsistent vs consistent | 1.46 (0.86-2.47) | 0.16 | | |
| No vs consistent | 1.71 (0.88-3.32) | 0.12 | | |
| HBV DNA at end of treatment (detected vs not detected) | 1.39 (0.95-2.03) | 0.09 | | |
| HBsAg at end of treatment (≥ 100 IU/mL vs < 100 IU/mL) | 2.77 (1.51-5.05) | <0.001 | 3.24 (1.75-6.01) | <0.001 |
| HBsAb at end of treatment (log mIU/mL) | 0.86 (0.63-1.18) | 0.35 | | |
| ALT at end of treatment (log U/L) | 1.04 (0.70-1.53) | 0.85 | | |
| AST at end of treatment (log U/L) | 1.02 (0.54-1.91) | 0.96 | | |
| HBV DNA at 1 month after stop treatment (log IU/mL) | 1.21 (1.15-1.27) | <0.001 | | |
| ALT at 1 month after stop treatment (log U/L) | 2.19 (1.60-3.01) | <0.001 | | |
| HBsAg at 1 month after stop treatment (log IU/mL) | 1.21 (1.08-1.35) | <0.001 | | |

To assess associations to sustained clinical response, logistic regression analysis was performed (see Table 15 in Example 2).

Statistical Analysis of Genetics Data: Considering that HBsAg status at the end of treatment and treatment regimen were predictive for onset of relapse, the association between the SNPs and onset of relapse was assessed using a Cox model¹ including HBsAg level at the last visit on treatment (high (≥ 100 IU/mL) vs. low (< 100 IU/mL)) and the antiviral treatment at enrollment (tenofovir vs. entecavir) as main effects.

All statistical analyses were executed within the R (3.6.1) statistical software. Statistical tests were two-sided and considered significant at the 0.05 significance level.

The genetic analysis set of markers associated with onset of relapse included 33 SNPs (see Tables 7 and 9 in Example 1) previously identified as significantly associated with onset of viral relapse on the one hand and 9 SNPs (see Tables 7 and 8) previously identified as significantly associated with onset of clinical relapse on the other hand, either by a candidate approach or a genetic scan (Table 24a). The 33 SNPs include 29 SNPs (see Table 9) identified as protective for early onset of viral relapse with an FDR<5%, 4 candidate SNPs (see Table 7) showing a significant association with the onset of viral relapse, and the 9 SNPs include 7 SNPs (see Table 8) identified as associated with onset of clinical relapse and 2 (see Table 7) candidate SNPs showing a significant association with onset of clinical relapse.

Table 24a shows the overview of the 42 SNPs described above. For each rsid, the closest gene (in chromosomal location) is indicated together with the origin of the SNP (reason for selection as candidate), and the corresponding minor and major alleles at this locus.

TABLE 24a

| RSID | GENE | GENETIC MODEL | ESTIMATE | P-VALUE | MAJOR ALLELE | MINOR ALLELE | RISK FACTOR | PROTECTIVE FACTOR | RESPONSE | ORIGIN |
|---|---|---|---|---|---|---|---|---|---|---|
| rs7534054 | CASZ1 | Additive | -1.04099 | 7.62E-07 | C | A | Presence of C | Presence of A allele | Time to VR | GWAS SCAN |
| rs4315565 | ANTXR1 | Additive | -0.63805 | 2.96E-06 | G | A | Presence of G | Presence of A allele | Time to VR | GWAS SCAN |
| rs12105972 | --- | Additive | 0.54109 | 5.40E-06 | G | C | Presence of C | Presence of G allele | Time to VR | GWAS SCAN |
| rs1994245 | TMEFF2 | Additive | 0.5708 | 2.78E-06 | T | C | Presence of C | Presence of T allele | Time to VR | GWAS SCAN |
| rs11896590 | TMEFF2 | Additive | -0.53883 | 4.77E-06 | A | G | Presence of A | Presence of G allele | Time to VR | GWAS SCAN |
| rs7629161 | LINC00877 | Additive | -0.81887 | 4.41E-07 | C | T | Presence of C | Presence of T allele | Time to VR | GWAS SCAN |
| rs9828024 | --- | Additive | -0.63531 | 3.51E-07 | A | G | Presence of A | Presence of G allele | Time to VR | GWAS SCAN |
| rs7670984 | GRID2 | Additive | 0.78041 | 5.36E-06 | C | A | Presence of A | Presence of C allele | Time to VR | GWAS SCAN |
| rs12645094 | NPY2R | Additive | 0.59411 | 2.41E-06 | A | C | Presence of C | Presence of A allele | Time to VR | GWAS SCAN |
| rs2163787 | SCGB3A2 | Additive | 0.84676 | 1.94E-06 | G | A | Presence of A | Presence of G allele | Time to VR | GWAS SCAN |
| rs924446 | --- | Additive | 0.56138 | 2.56E-06 | C | T | Presence of T | Presence of C allele | Time to VR | GWAS SCAN |
| rs180001 | ATXN1 | Additive | -0.6157 | 1.32E-06 | A | G | Presence of A | Presence of G allele | Time to VR | GWAS SCAN |
| rs12199613 | BTN3A2 | Additive | 0.68844 | 9.98E-07 | C | T | Presence of T | Presence of C allele | Time to VR | GWAS SCAN |
| rs2394952 | HLA-C | Additive | 0.72804 | 6.66E-07 | A | G | Presence of G | Presence of A allele | Time to VR | GWAS SCAN |
| rs17152258 | CDHR3 | Additive | 0.68058 | 4.49E-06 | C | T | Presence of T | Presence of C allele | Time to VR | GWAS SCAN |
| rs7459445 | --- | Additive | 0.9052 | 8.94E-07 | T | C | Presence of C | Presence of T allele | Time to VR | GWAS SCAN |
| rs1053403 | SEC61A2 | Additive | -0.5467 | 2.58E-06 | A | G | Presence of A | Presence of G allele | Time to VR | GWAS SCAN |
| rs2767035 | PDHX | Additive | -0.64992 | 3.22E-07 | T | C | Presence of T | Presence of C allele | Time to VR | GWAS SCAN |
| rs3943102 | | Additive | 0.59123 | 1.69E-06 | C | T | Presence of T | Presence of C allele | Time to VR | GWAS SCAN |
| rs2154237 | FUT8 | Additive | -1.05463 | 1.65E-06 | T | G | Presence of T | Presence of G allele | Time to VR | GWAS SCAN |
| rs73371840 | C14orf80 | Additive | -0.565 | 5.20E-06 | T | C | Presence of T | Presence of C allele | Time to VR | GWAS SCAN |
| rs7205040 | PRKCB | Additive | -0.64123 | 4.86E-06 | C | T | Presence of C | Presence of T allele | Time to VR | GWAS SCAN |
| rs552219 | LAMA1 | Additive | -0.78739 | 1.28E-06 | G | A | Presence of G | Presence of A allele | Time to VR | GWAS SCAN |
| rs78045374 | | Additive | 0.75648 | 3.47E-06 | T | C | Presence of C | Presence of T allele | Time to VR | GWAS SCAN |
| rs117634357 | TP73 | Recessive | 1.26001 | 1.18E-07 | C | T | Presence of TT genotype | Presence of C allele | Time to VR | GWAS SCAN |
| rs2236895 | LAMB3 | Recessive | 1.29814 | 5.26E-07 | G | T | Presence of TT genotype | Presence of G allele | Time to VR | GWAS SCAN |
| rs7646021 | CADPS | Recessive | 1.01408 | 5.35E-07 | T | G | Presence of GG genotype | Presence of T allele | Time to VR | GWAS SCAN |
| rs17152247 | CDHR3 | Recessive | 0.97425 | 4.50E-07 | C | T | Presence of TT genotype | Presence of C allele | Time to VR | GWAS SCAN |
| rs10235518 | LSM8 | Recessive | -1.69479 | 9.99E-07 | T | C | Presence of TT genotype | Presence of C allele | Time to VR | GWAS SCAN |
| rs4668818 | LINC00276 | Recessive | 1.35383 | 8.47E-07 | T | C | Presence of CC genotype | Presence of T allele | Time to CR | GWAS SCAN |
| rs948006 | WNT11 | Recessive | 1.18847 | 4.25E-07 | C | T | Presence of TT genotype | Presence of C allele | Time to CR | GWAS SCAN |
| rs2934456 | SNX29P2 | Recessive | 1.35077 | 2.68E-07 | A | G | Presence of GG genotype | Presence of A allele | Time to CR | GWAS SCAN |
| rs77586835 | | Additive | 0.83099 | 3.30E-06 | T | C | Presence of C allele | Presence of T allele | Time to CR | GWAS SCAN |
| rs75876539 | LINC00867 | Additive | 0.86924 | 2.09E-06 | C | A | Presence of A allele | Presence of C allele | Time to CR | GWAS SCAN |
| rs8050261 | RBFOX1 | Additive | 0.85877 | 2.95E-06 | G | C | Presence of C allele | Presence of G allele | Time to CR | GWAS SCAN |
| rs1542951 | | Additive | -0.66951 | 4.89E-06 | C | A | Presence of C allele | Presence of A allele | Time to CR | GWAS SCAN |
| rs231770 | CTLA4 | Additive | -0.29996 | 0.03465 | T | C | Presence of T allele | Presence of C allele | Time to CR | CANDIDATE SCAN |
| rs9277535 | HLA-DPB1 | Additive | -0.37286 | 0.03716 | G | A | Presence of G allele | Presence of A allele | Time to CR | CANDIDATE SCAN |
| rs3130542 | HLA-C | Additive | 0.72804 | 6.66E-07 | G | A | Presence of A allele | Presence of G allele | Time to VR | CANDIDATE SCAN |
| rs7574865 | STAT4 | Dominant | -0.32362 | 0.04844 | G | T | Presence of GG genotype | Presence of T allele | Time to VR | CANDIDATE SCAN |
| rs2296651 | SLC10A1 | Additive | -0.6226 | 0.024 | G | A | Presence of G allele | Presence of A allele | Time to VR | CANDIDATE SCAN |
| rs1419881 | TCF19 | Additive | 0.26178 | 0.02037 | A | G | Presence of G allele | Presence of A allele | Time to VR | CANDIDATE SCAN |

The genetic analysis set of markers associated with sustained clinical response included 24 SNPs significantly associated with sustained clinical response (also see Table 17 and its description in Example 2), either by a candidate approach or as associated with onset of viral relapse, taking into account HBsAg as main effect (see Table 24b below).

TABLE 24b

| | | | | | | | | | | | | | |
|---|---|---|---|---|---|---|---|---|---|---|---|---|---|
| RSID | GENE | MINOR ALLELE | MAJOR ALLELE | MAF | 0 | 1 | 2 | ALLELES | Model | estimate | p.value | ALLELE PROTECTIVE | ALLELE RISK |
| rs7534054 | CASZ1 | A | C | 0.1236 | CC | AC | AA | CC (0) - AC (1) - AA (2) | ADD | 2.009565 | 1.98E-05 | A | C |
| rs180001 | ATXN1 | G | A | 0.3564 | AA | GA | GG | AA (0) - GA (1) - GG (2) | ADD | 1.544927 | 9.91E-05 | G | A |
| rs4315565 | ANTXR1 | A | G | 0.3114 | GG | AG | AA | GG (0) - AG (1) - AA (2) | ADD | 1.509224 | 0.000155 | A | G |
| rs2154237 | FUT8 | G | T | 0.1077 | TT | GT | GG | TT (0) - GT (1) - GG (2) | ADD | 1.874174 | 0.000155 | G | T |
| rs10235518 | LSM8 | C | T | 0.3333 | TT | CT | CC | TT (0) - CT (1) - CC (2) | REC | 2.052922 | 0.000223 | CC | T |
| rs9828024 | | G | A | 0.4503 | AA | GA | GG | AA (0) - GA (1) - GG (2) | ADD | 1.412379 | 0.000239 | G | A |
| rs924446 | | T | C | 0.3874 | CC | TC | TT | CC (0) - TC (1) - TT (2) | ADD | -1.87341 | 0.000282 | C | T |
| rs12105972 | | C | G | 0.4011 | GG | CG | CC | GG (0) - CG (1) - CC (2) | ADD | -1.6977 | 0.000378 | G | C |
| rs2767035 | PDHX | C | T | 0.3626 | TT | CT | CC | TT (0) - CT (1) - CC (2) | ADD | 1.323577 | 0.000453 | C | T |
| rs7205040 | PRKCB | T | C | 0.3022 | CC | TC | TT | CC (0) - TC (1) - TT (2) | ADD | 1.196834 | 0.000721 | T | C |
| rs3943102 | | T | C | 0.3934 | CC | TC | TT | CC (0) - TC (1) - TT (2) | ADD | -1.49203 | 0.000948 | C | T |
| rs12645094 | NPY2R | C | A | 0.4724 | AA | CA | CC | AA (0) - CA (1) - CC (2) | ADD | -1.34657 | 0.001003 | A | C |
| rs73371840 | C14orf80 | C | T | 0.3791 | TT | CT | CC | TT (0) - CT (1) - CC (2) | ADD | 1.061706 | 0.002803 | C | T |
| rs7629161 | LINC00877 | T | C | 0.2444 | CC | TC | TT | CC (0) - TC (1) - TT (2) | ADD | 1.057212 | 0.003809 | T | C |
| rs1053403 | SEC61A2 | G | A | 0.4725 | AA | GA | GG | AA (0) - GA (1) - GG (2) | ADD | 1.010636 | 0.00456 | G | A |
| rs552219 | LAMA1 | A | G | 0.2167 | GG | AG | AA | GG (0) - AG (1) - AA (2) | ADD | 1.063744 | 0.005655 | A | G |
| rs2296651 | SLC10A1 | A | G | 0.05769 | GG | AG | #N/A | GG (0) - AG (1) | ADD | 1.659699 | 0.005866 | A | G |
| rs3130542 | HLA-C | A | G | 0.2225 | GG | AG | AA | GG (0) - AG (1) - AA (2) | ADD | -1.73127 | 0.006898 | G | A |
| rs2394952 | HLA-C | G | A | 0.2225 | AA | GA | GG | AA (0) - GA (1) - GG (2) | ADD | -1.73127 | 0.006898 | A | G |
| rs11896590 | TMEFF2 | G | A | 0.4423 | AA | GA | GG | AA (0) - GA (1) - GG (2) | ADD | 0.949445 | 0.008185 | G | A |
| rs17152258 | CDHR3 | T | C | 0.2088 | CC | TC | TT | CC (0) - TC (1) - TT (2) | ADD | -1.94637 | 0.009913 | C | T |
| rs1994245 | TMEFF2 | C | T | 0.2983 | TT | CT | CC | TT (0) - CT (1) - CC (2) | ADD | -1.12437 | 0.01493 | T | C |
| rs12199613 | BTN3A2 | T | C | 0.2418 | CC | TC | TT | CC (0) - TC (1) - TT (2) | ADD | -1.30741 | 0.019419 | C | T |
| rs7459445 | | C | T | 0.1346 | TT | CT | #N/A | TT (0) - CT (1) | ADD | -2.15637 | 0.038937 | T | C |

Both for clinical and virological relapse, different Cox proportional hazard regression models were compared by AIC, BIC (Yang Y., Biometrika. 2005, 92(4):937-950), and concordance (Harrell’s C-index (Harrell F. E., The Journal of the American Medical Association. 1982, 247(18):2543)) to assess the association to the hazard of relapse. In addition, for the most relevant models, time-dependent receiver operating characteristic (ROC (Heagerty P. J., et al., Biometrics, 2000, 56(2):337-344)) curves (and the corresponding area under the curve (AUC)) were compared. On the full set of hit SNP, a stepdown approach was applied to obtain a minimal set of significant SNPs. Main effects for end of treatment HBsAg and treatment regimen are always included.

For the sustained clinical response analysis, logistic regression was applied to assess the association to the odds of sustained clinical response within follow-up after stop treatment. Different models were compared by AIC, BIC, and ROC AUC. Main effects for end of treatment HBsAg are always included.

### Results

Study Population: Table 14 in Example 2 provides an overview of the clinical characteristics of the study population.

Out of the 186 patients included in the cohort, 161 (86.6%) experienced a virological relapse (FIG. 30A), of whom 110 (59.1%) also had a clinical relapse (FIG. 30B).

Clinical Relapse: The multivariate Cox proportional hazard regression analysis reveals that older age (HR, 1.02; 95% CI, 1.00-1.04; P = 0.04), tenofovir treatment (compared to entecavir treatment) (HR, 1.80; 95% CI, 1.21-2.68; P = 0.004), and high HBsAg at the end of treatment (HR, 3.06; 95% CI, 1.64-5.69; P < 0.001), increase the hazard to clinical relapse and are therefore negatively associated to clinical relapse (Table 23a). An HBsAg level of 100 IU/mL was used as threshold to differentiate low and high end of treatment HBsAg, corresponding to 89.09% sensitivity and 35.53% specificity for predicting clinical relapse.

HBV DNA, HBsAg, and ALT at 1 month after stop treatment are additional variables showing a significant association to clinical relapse in the univariate models. These covariates were however excluded from the multivariate model as HBV DNA and ALT at 1 month after stop treatment are highly associated to the treatment regimen and HBsAg 1 month after stop treatment highly associated to end of treatment HBsAg (r = 0.93, P < 0.001).

Among the 110 clinical relapsers, the onset of clinical relapse is on average 100 days later after stopping entecavir treatment compared to tenofovir treatment. The time to clinical relapse in the end of treatment (EOT) HBsAg high group is on average 60 days later compared to the low group. This is probably due to 7 of the 12 patients with low EOT HBsAg levels being in the tenofovir group (compared to 5 in the entecavir group).

Virological Relapse: The multivariate Cox proportional hazard regression analysis reveals that older age (HR, 1.02, 95% CI, 1.00-1.03; P =0.03), prior HBeAg negativity at the start of NA therapy (compared to acquiring HBeAg negativity during treatment) (HR, 2.20; 95% CI, 1.43-3.40; P < 0.001), tenofovir treatment (compared to entecavir treatment) (HR, 2.97; 95% CI, 2.10-4.20; P < 0.001), and high HBsAg at the end of treatment (HR, 2.37; 95% CI, 1.51-3.74; P < 0.001), increase the hazard to virological relapse and are therefore negatively associated to virological relapse (Table 23b).

HBV DNA, HBsAg, and ALT at 1 month after treatment cessation were again excluded from the multivariate model due to strong association to the treatment regimen and end of treatment HBsAg. Finally, baseline HBeAg is associated to age (P < 0.001) and gender (P = 0.03), and therefore not included in the models including genetic markers. The effects of gender and age were moderate in a relatively unbalanced cohort, and HBeAg status is more reflecting the chronic stage of the patients.

Genetic signature associated with onset of clinical relapse: HBsAg at the last visit on treatment and treatment regimen were considered as main effects in the multivariate model. Six out of the 9 SNPs identified as protective for early clinical relapse are identified, applying the stepdown approach, as improving prediction of onset of clinical relapse in a multivariate model: rs4668818, rs948006, rs2934456, rs75876539, rs8050261 and rs1542951 (referred as ‘Clinical signature SNPs’). Estimated hazard ratios are reported for both set of SNPs taking into account HBsAg level and treatment regimen as main effects in Table 26a and Table 26b. Performance of the multivariate models not including the SNPs, including only the Clinical signature SNPs, and including all nine markers are compared by the AUC of the ROC curves predicting onset of clinical relapse (Table 25) and show outperformance of the model including the Clinical signature SNPs to the model not including the SNPs. Table 25 compared the performances between a multivariate model including HBsAg and regimen as main effects, adding the nine identified SNPs and a subset of six Clinical signature SNPs to predict the onset of clinical relapse (AIC, BIC, concordance and ROC AUC). No clear additional improvement is observed comparing the model including all SNPs to the model including only the Clinical signature SNPs.

TABLE 25

| Model | AIC | BIC | Concordance | ROC AUC |
|---|---|---|---|---|
| HBsAg + regimen | 958.32 | 966.20 | 0.63 | 0.67 |
| HBsAg + regimen + all SNP | 871.30 | 902.80 | 0.81 | 0.89 |
| HBsAg + regimen + clinical signature SNPs | 872.96 | 896.58 | 0.80 | 0.86 |

TABLE 26a

Cox proportional hazard regression analysis for clinical relapse including all nine SNPs identified from a univariate analysis

| Covariate | rsid | HR | Lower | Upper | Clinical Signature SNPs |
|---|---|---|---|---|---|
| HBsAg at end of treatment (high vs low) | | 2.77 | 1.44 | 5.32 | |
| Treatment regimen (tenofovir vs entecavir) | | 1.75 | 1.13 | 2.71 | |
| Treatment regimen (other vs entecavir) | | 1.81 | 0.79 | 4.15 | |
| rs4668818 / LINC00276 (REC, C vs T) | rs4668818 | 3.13 | 1.71 | 5.73 | yes |
| rs948006 / WNT11 (REC, T vs C) | rs948006 | 2.64 | 1.54 | 4.52 | yes |
| rs2934456 / SNX29P2 (REC, G vs A) | rs2934456 | 4.24 | 2.42 | 7.42 | yes |
| rs77586835 / - (ADD, C vs T) | rs77586835 | 1.42 | 0.96 | 2.11 | no |
| rs75876539 / LINC00867 (ADD, A vs C) | rs75876539 | 2.19 | 1.46 | 3.28 | yes |
| rs8050261 / RBFOX1 (ADD, C vs G) | rs8050261 | 2.03 | 1.37 | 3.01 | yes |
| rs1542951 / - (ADD, A vs C) | rs1542951 | 0.67 | 0.48 | 0.94 | yes |
| rs231770 / CTLA4 (ADD, C vs T) | rs231770 | 0.81 | 0.59 | 1.10 | no |
| rs9277535 / HLA-DPB1 (ADD, A vs G) | rs9277535 | 0.72 | 0.49 | 1.05 | no |

TABLE 26b

Cox proportional hazard regression analysis for clinical relapse including the Clinical SNP signature

| Covariate | rsid | HR | Lower | Upper |
|---|---|---|---|---|
| HBsAg at end of treatment (high vs low) | | 2.77 | 1.44 | 5.32 |
| Treatment regimen (tenofovir vs entecavir) | | 1.89 | 1.23 | 2.90 |
| Treatment regimen (other vs entecavir) | | 1.98 | 0.87 | 4.50 |
| rs4668818 / LINC00276 (REC, C vs T) | rs4668818 | 3.52 | 2.03 | 6.10 |
| rs948006 / WNT11 (REC, T vs C) | rs948006 | 2.81 | 1.68 | 4.69 |
| rs2934456 / SNX29P2 (REC, G vs A) | rs2934456 | 4.08 | 2.33 | 7.13 |
| rs75876539 / LINC00867 (ADD, A vs C) | rs75876539 | 2.47 | 1.67 | 3.65 |
| rs8050261 / RBFOX1 (ADD, C vs G) | rs8050261 | 2.01 | 1.36 | 2.97 |
| rs1542951 / - (ADD, A vs C) | rs1542951 | 0.62 | 0.45 | 0.86 |

The AUC of the time-dependent ROC curves, assessed at two years after stop treatment, corresponding to the models including the nine evaluated markers and the clinical signature with six out of the nine genetic markers equal 0.89 and 0.86, compared to 0.67 for the model including end of treatment HBsAg and treatment regimen alone (FIG. 31, Table 25). As shown in FIG. 31, the ROC curve is a full line with AUC 0.67 corresponding to the model including HBsAg level at the end of treatment and treatment regimen, a dotted line with AUC 0.86 corresponding to the curve adding on top the six Clinical signature SNPs, and a dashed line with AUC 0.89 corresponding to the curve of nine SNPs identified in the univariate analysis.

The model including the Clinical signature SNPs outperforms the model including all nine SNPs, considering the BIC criteria with very similar AIC and concordance.

Genetic signature associated with onset of viral relapse: HBsAg at the last visit on treatment and treatment regimen were considered as main effects in the multivariate model. 17 out of the 33 SNPs identified as protective for early viral relapse are identified, applying the stepdown approach, improving prediction of onset of viral relapse in a multivariate model. The overview of estimated hazard ratios and corresponding confidence intervals for all 33 SNPs is reported in Table 27a. The overview of the model including the 17 SNPs (referred as ‘Virological signature SNPs) together with HBsAg and treatment regimen is reported in Table 27b and FIG. 33.

Tables 27a and 27b show the cox proportional hazard regression analysis for virological relapse including all 33 SNPs (Table 27a) and Virological signature SNPs (Table 27b). For each SNP, its rsid is indicated together with the corresponding model (ADD = additive, DOM = dominant, REC = recessive). The sign of the estimate indicates the risk/protective alleles.

FIG. 33 shows the overview of the estimated hazard ratios and corresponding 95% confidence intervals for the Cox proportional hazard regression model including the Virological signature SNPs.

TABLE 27a

| Covariate | RSID | HR | Lower | Upper | Virological Signature SNPs |
|---|---|---|---|---|---|
| HBsAg at end of treatment (high vs low) | | 1.01 | 0.56 | 1.82 | |
| Treatment regimen (tenofovir vs entecavir) | | 7.26 | 3.83 | 13.78 | |
| Treatment regimen (other vs entecavir) | | 4.32 | 1.69 | 11.00 | |
| rs7534054 / CASZ1 (ADD, A vs C) | rs7534054 | 0.54 | 0.30 | 0.97 | yes |
| rs4315565 / ANTXR1 (ADD, A vs G) | rs4315565 | 0.72 | 0.51 | 1.03 | no |
| rs12105972 / - (ADD, C vs G) | rs12105972 | 1.32 | 0.95 | 1.82 | yes |
| rs1994245 / TMEFF2 (ADD, C vs T) | rs1994245 | 1.43 | 0.97 | 2.11 | no |
| rs11896590 / TMEFF2 (ADD, G vs A) | rs11896590 | 1.47 | 0.97 | 2.23 | no |
| rs7629161 / LINC00877 (ADD, T vs C) | rs7629161 | 0.64 | 0.43 | 0.95 | yes |
| rs9828024 / - (ADD, G vs A) | rs9828024 | 0.64 | 0.46 | 0.90 | yes |
| rs7670984 / GRID2 (ADD, A vs C) | rs7670984 | 1.91 | 1.21 | 3.02 | yes |
| rs12645094 / NPY2R (ADD, C vs A) | rs12645094 | 1.23 | 0.89 | 1.70 | yes |
| rs2163787 / SCGB3A2 (ADD, A vs G) | rs2163787 | 1.61 | 1.04 | 2.50 | yes |
| rs924446 / - (ADD, T vs C) | rs924446 | 1.36 | 0.99 | 1.85 | yes |
| rs180001 / ATXN1 (ADD, G vs A) | rs180001 | 0.81 | 0.58 | 1.13 | no |
| rs12199613 / BTN3A2 (ADD, T vs C) | rs12199613 | 1.53 | 1.04 | 2.26 | no |
| rs2394952 / HLA-C (ADD, G vs A) | rs2394952 | 0.84 | 0.55 | 1.30 | no |
| rs17152258 / CDHR3 (ADD, T vs C) | rs17152258 | 1.06 | 0.72 | 1.57 | no |
| rs7459445 / - (ADD, C vs T) | rs7459445 | 1.50 | 0.94 | 2.40 | no |
| rs1053403 / SEC61A2 (ADD, G vs A) | rs1053403 | 0.66 | 0.49 | 0.90 | yes |
| rs2767035 / PDHX (ADD, C vs T) | rs2767035 | 0.61 | 0.44 | 0.86 | yes |
| rs3943102 / - (ADD, T vs C) | rs3943102 | 1.31 | 0.95 | 1.81 | yes |
| rs2154237 / FUT8 (ADD, G vs T) | rs2154237 | 0.62 | 0.35 | 1.11 | no |
| rs73371840 / C14orf80 (ADD, C vs T) | rs73371840 | 1.10 | 0.81 | 1.51 | no |
| rs7205040 / PRKCB (ADD, T vs C) | rs7205040 | 0.84 | 0.60 | 1.19 | no |
| rs552219 / LAMA1 (ADD, A vs G) | rs552219 | 0.97 | 0.64 | 1.47 | no |
| rs78045374 / - (ADD, C vs T) | rs78045374 | 1.51 | 0.96 | 2.39 | no |
| rs117634357 / TP73 (REC, T vs C) | rs117634357 | 2.81 | 1.47 | 5.37 | yes |
| rs2236895 / LAMB3 (REC, T vs G) | rs2236895 | 2.64 | 1.28 | 5.44 | yes |
| rs7646021 / CADPS (REC, G vs T) | rs7646021 | 2.92 | 1.72 | 4.96 | yes |
| rs17152247 / CDHR3 (REC, T vs C) | rs17152247 | 1.76 | 1.06 | 2.93 | yes |
| rs10235518 / LSM8 (REC, C vs T) | rs10235518 | 0.34 | 0.13 | 0.84 | yes |
| not applicable (overlaps completely with rs2394952) | rs3130542 | NA | NA | NA | not applicable (overlaps completely with rs2394952) |
| rs7574865 / STAT4 (DOM, T vs G) | rs7574865 | 0.89 | 0.57 | 1.37 | no |
| rs2296651 / SLC10A1 (ADD, A vs G) | rs2296651 | 0.87 | 0.43 | 1.78 | no |
| rs1419881 / TCF19 (ADD, G vs A) | rs1419881 | 1.61 | 1.17 | 2.24 | yes |

TABLE 27b

| Covariate | rsid | HR | Lower | Upper |
|---|---|---|---|---|
| HBsAg at end of treatment (high vs low) | | 0.89 | 0.52 | 1.50 |
| Treatment regimen (tenofovir vs entecavir) | | 7.06 | 4.14 | 12.04 |
| Treatment regimen (other vs entecavir) | | 4.89 | 2.19 | 10.89 |
| rs7534054 / CASZ1 (ADD, A vs C) | rs7534054 | 0.51 | 0.31 | 0.83 |
| rs12105972 / - (ADD, C vs G) | rs12105972 | 1.55 | 1.17 | 2.07 |
| rs7629161 / LINC00877 (ADD, T vs C) | rs7629161 | 0.61 | 0.42 | 0.88 |
| rs9828024 / - (ADD, G vs A) | rs9828024 | 0.62 | 0.47 | 0.84 |
| rs7670984 / GRID2 (ADD, A vs C) | rs7670984 | 1.90 | 1.27 | 2.85 |
| rs12645094 / NPY2R (ADD, C vs A) | rs12645094 | 1.34 | 1.01 | 1.79 |
| rs2163787 / SCGB3A2 (ADD, A vs G) | rs2163787 | 1.64 | 1.10 | 2.47 |
| rs924446 / - (ADD, T vs C) | rs924446 | 1.42 | 1.06 | 1.91 |
| rs1053403 / SEC61A2 (ADD, G vs A) | rs1053403 | 0.67 | 0.51 | 0.88 |
| rs2767035 / PDHX (ADD, C vs T) | rs2767035 | 0.60 | 0.43 | 0.82 |
| rs3943102 / - (ADD, T vs C) | rs3943102 | 1.50 | 1.14 | 1.97 |
| rs117634357 / TP73 (REC, T vs C) | rs117634357 | 3.04 | 1.72 | 5.38 |
| rs2236895 / LAMB3 (REC, T vs G) | rs2236895 | 2.89 | 1.50 | 5.57 |
| rs7646021 / CADPS (REC, G vs T) | rs7646021 | 3.05 | 1.89 | 4.92 |
| rs17152247 / CDHR3 (REC, T vs C) | rs17152247 | 1.60 | 1.02 | 2.51 |
| rs10235518 / LSM8 (REC, C vs T) | rs10235518 | 0.23 | 0.10 | 0.51 |
| rs1419881 / TCF19 (ADD, G vs A) | rs1419881 | 1.56 | 1.20 | 2.04 |

Performance of the multivariate models including HBsAg at the end of treatment and treatment regimen alone, in addition the 17 Virological signature SNPs, including all 33 genetic markers, and including a subset of two specific genetic markers (rs2154237 and rs2296651, functional SNPs in respectively FUT8 and SLC10A1 genes, described as having functional consequence on the NTCP receptor) are compared by AUC of the time-dependent receiver operating characteristic curves (time point two years after stop treatment), predicting onset of clinical relapse (Table 28). This comparison highlights the outperformance of the Virological signature SNPs model over the model not including any genetic marker. No clear additional improvement is observed comparing the model including all SNPs to the model including only the Virological signature SNPs (especially considering BIC) The model including two SNPs in FUT8 and SLC10A1 outperformed the model non including any genetic marker. The ROC AUC corresponding to the models including the 33 evaluated markers and the subset of virological signature markers equal 0.98 and 0.97, compared to 0.69 for the model including end of treatment HBsAg and treatment regimen alone (FIG. 5). The model including the two functional SNPs in SLC10A1 and FUT8 corresponds to an AUC of 0.79.

TABLE 28

| Model | AIC | BIC | Concordance | AUC |
|---|---|---|---|---|
| HBsAg + regimen | 1183.61 | 1192.40 | 0.66 | 0.69 |
| HBsAg + regimen + all SNP | 1017.94 | 1120.39 | 0.87 | 0.98 |
| HBsAg + regimen + Virological Signature SNPs | 1007.95 | 1066.50 | 0.86 | 0.97 |
| HBsAg + regimen + FUT8 + SLC10A1 | 1166.03 | 1180.66 | 0.70 | 0.79 |

Sustained Clinical Response: Logistic regression shows the negative association of prior HBeAg negativity at the start of NA therapy (OR, 0.25; 95% CI, 0.06-0.99; P = 0.05), high HBsAg at the end of treatment (OR, 0.16; 95% CI, 0.04-0.55; P = 0.005), and higher HBV DNA 1 month after stop treatment (OR, 0.54; 95% CI, 0.28-0.78; P = 0.01), to sustained clinical response (see Table 15 in Example 2).

### Genetic Signature Predictive of Sustained Clinical Response

HBsAg at the last visit on treatment was considered as main effect in the multivariate model. Out of the 24 SNPs (see Table 17, FIG. 28, and the discussion in Example 2) identified previously as significantly associated with sustained clinical response. For further analysis, one SNP was removed due to high pairwise correlation, and eight other SNPs were not considered in the multivariate model due to high variance inflation factors (indicating high between SNP collinearity). Thresholds were set at 0.8 and 10 for the correlation and the variance inflation factor respectively. In total 15 SNPs were considered for the multivariate signature evaluation.

Applying the stepdown approach, six genetic markers were identified as improving prediction of sustained clinical response in a multivariate model: rs4315565, rs2154237, rs9828024, rs12105972, rs3943102 and rs2296651 (referred as ‘SCR signature SNPs’). Odds ratios are reported for both set of SNPs taking into account HBsAg level as main effect on Table 28a and Table 28b and FIG. 35.

Tables 29a-b show the logistic regression analysis for sustained clinical response including all 15 SNPs identified in a univariate analysis (Table 29a) and SCR signature SNP (Table 29b). For each SNP, its rsid is indicated together with the corresponding model (ADD = additive, DOM = dominant, REC = recessive). The sign of the estimate indicates the risk/protective alleles.

FIG. 35 shows the overview of the estimated odds ratios and corresponding 95% confidence intervals for the logistic regression model including the SCR signature SNPs.

TABLE 29a

| Covariate | rsid | OR | Lower | Upper | Sustained Clinical Response Signature |
|---|---|---|---|---|---|
| HBsAg at end of treatment (high vs low) | | 0.02 | 0.00 | 0.46 | |
| rs180001 / ATXN1 (ADD, G vs A) | rs180001 | 5.33 | 0.23 | 365.35 | no |
| rs4315565 / ANTXR1 (ADD, A vs G) | rs4315565 | 74.96 | 3.22 | 71843.63 | yes |
| rs2154237 / FUT8 (ADD, G vs T) | rs2154237 | 22.38 | 0.26 | 6221.56 | yes |
| rs10235518 / LSM8 (REC, C vs T) | rs10235518 | 1.81 | 0.03 | 146.69 | no |
| rs9828024 / - (ADD, G vs A) | rs9828024 | 16.73 | 0.91 | 2168.93 | yes |
| rs12105972 / - (ADD, C vs G) | rs12105972 | 0.05 | 0.00 | 0.92 | yes |
| rs7205040 / PRKCB (ADD, T vs C) | rs7205040 | 16.90 | 0.97 | 4024.14 | no |
| rs3943102 / - (ADD, T vs C) | rs3943102 | 0.03 | 0.00 | 0.61 | yes |
| rs12645094 / NPY2R (ADD, C vs A) | rs12645094 | 0.35 | 0.01 | 7.52 | no |
| rs73371840 / C14orf80 (ADD, C vs T) | rs73371840 | 3.71 | 0.21 | 225.87 | no |
| rs1053403 / SEC61A2 (ADD, G vs A) | rs1053403 | 9.01 | 0.80 | 342.98 | no |
| rs2296651 / SLC10A1 (ADD, A vs G) | rs2296651 | 71.38 | 0.87 | 244733.50 | yes |
| rs17152258 / CDHR3 (ADD, T vs C) | rs17152258 | 0.05 | 0.00 | 1.48 | no |
| rs12199613 / BTN3A2 (ADD, T vs C) | rs12199613 | 0.30 | 0.00 | 6.76 | no |
| rs7459445 / - (ADD, C vs T) | rs7459445 | 0.01 | 0.00 | 2.89 | no |

TABLE 29b

| Covariate | Variable | OR | Lower | Upper |
|---|---|---|---|---|
| HBsAg at end of treatment (high vs low) | | 0.04 | 0.00 | 0.27 |
| rs4315565 / ANTXR1 (ADD, A vs G) | rs4315565 | 17.50 | 3.61 | 168.10 |
| rs2154237 / FUT8 (ADD, G vs T) | rs2154237 | 10.88 | 1.26 | 144.15 |
| rs9828024 / - (ADD, G vs A) | rs9828024 | 22.48 | 4.55 | 225.96 |
| rs12105972 / - (ADD, C vs G) | rs12105972 | 0.08 | 0.01 | 0.43 |
| rs3943102 / - (ADD, T vs C) | rs3943102 | 0.04 | 0.00 | 0.22 |
| rs2296651 / SLC10A1 (ADD, A vs G) | rs2296651 | 19.81 | 2.06 | 290.92 |

Performance of the multivariate models not including the SNPs, including only the SCR signature SNPs and including all 15 markers are compared by the AUC of the ROC curves predicting sustained clinical response (Table 30, FIG. 36), and show outperformance of the model including the SCR signature SNPs to the model not including the SNPs.

Table 30 compared the performances between a multivariate model including HBsAg as main effect, adding the 15 identified SNPs and a subset of six SCR signature SNPs to predict sustained clinical response: AIC, BIC, and ROC AUC.

TABLE 30

| Model | AIC | BIC | AUC |
|---|---|---|---|
| HBsAg | 124.04 | 130.29 | 0.66 |
| HBsAg + all SNP | 56.59 | 109.69 | 0.99 |
| HBsAg + SCR signature SNP | 57.08 | 82.07 | 0.96 |

FIG. 36 shows the ROC curve corresponding to the model including HBsAg level at the end of treatment (AUC 0.66), adding the six SCR signature SNPs (AUC 0.97), including the 15 SNPs previously associated with sustained clinical response (AUC 0.99). No clear additional improvement is observed comparing the model including all SNPs to the model including only the SCR signature SNPs. The ROC AUC of the models including the 15 evaluated markers and the SCR signature with six out of the 15 genetic markers equal 0.97 and 0.99, compared to 0.66 for the model including end of treatment HBsAg alone.

### Example 4. Validation Cohort

In an independent study, chronic hepatis B HBeAg negative patients are enrolled in their last year of a minimum of three years treatment regimen of direct antiviral treatment (nucleoside analogs, or NUC). Patients are followed up for up to two years after treatment discontinuation. DNA are collected for all subjects.

This cohort is informative to confirm the predictivity of specific genetic markers (e.g., HLA, SNPs identified already) for onset of virological relapse (HBV DNA>2000 IU/ml), clinical relapse (virological relapse and ALT increase above two times upper limit of normal) and sustained clinical response (no virological relapse during the entire follow up period).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated set of probes for use in treating a chronic hepatitis B (CHB) infection in a subject in need thereof, wherein the set of probes detects a panel of single nucleotide polymorphisms (SNPs), and the panel comprises one or more SNPs associated with time to relapse, and the one or more SNPs are selected from the group consisting of rs2154237, rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881.

2-8. (canceled)

9. The isolated set of probes for use of claim 1, wherein the SNP is rs2296651 or rs231770.

10. (canceled)

11. The isolated set of probes for use of claim 1, wherein the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, allele C in rs10235518, allele T in rs4668818, allele C in rs948006, allele A in rs2934456, allele T in rs77586835, allele C in rs75876539, allele G in rs8050261, or allele A in rs1542951.

12-13. (canceled)

14. The isolated set of probes for use of claim 1, wherein the treatment further comprises measuring the level of at least one of HBV DNA, alanine aminotransferase (ALT), and hepatitis B e-antigen (HBeAg) in a biological sample of the subject.

15. The isolated set of probes for use of claim 1, wherein if the panel of the one or more SNPs is detected in the biological sample, the treatment comprises:
(1) treating the subject with a therapeutically effective amount of a nucleotide or nucleoside analogue (NUC) two years or later after the discontinuation of the NUC treatment,
(2) continuing treating the subject with the NUC until CHB infection is suppressed in the subject; or
(3) monitoring relapse in the subject two years or later after the discontinuation of the NUC treatment.

16. The isolated set of probes for use of claim 1, wherein if none of the one or more SNPs is detected in the biological sample, the treatment comprises:
(1) monitoring relapse in the subject prior to two years after the discontinuation of the NUC treatment;
(2) administering to the subject a therapeutically effective amount of a non-NUC agent; or
(3) switching from the NUC treatment to a non-NUC treatment.

17. The isolated set of probes for use of claim 15, wherein the NUC is selected from the group consisting of tenofovir, entecavir, lamivudine, adefovir, and telbivudine.

18. The isolated set of probes for use of claim 16, wherein the non-NUC agent is interferon.

19-24. (canceled)

25. An isolated set of probes capable of detecting a panel of SNPs, and the panel comprises one or more SNPs associated with time to relapse, and the one or more SNPs are selected from the group consisting of rs2154237, rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881.

26-34. (canceled)

35. The isolated set of probes of claim 25, wherein the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, allele C in rs10235518, allele T in rs4668818, allele C in rs948006, allele A in rs2934456, allele T in rs77586835, allele C in rs75876539, allele G in rs8050261, or allele A in rs1542951.

36-37. (canceled)

38. An in vitro method of monitoring relapse of a chronic hepatitis B (CHB) infection in a subject, wherein the method comprises:
a. administering to the subject a therapeutically effective amount of a nucleotide or nucleoside analogue (NUC) to treat the CHB infection;
b. discontinuing the NUC treatment when the CHB infection is suppressed in the subject;
c. detecting in a biological sample obtained from the subject a panel of single nucleotide polymorphisms (SNPs), and the panel comprises one or more SNPs associated with time to relapse, and the one or more SNPs are selected from the group consisting of rs2154237, rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881; and
d. monitoring the relapse in the subject two years or later after the discontinuation of the NUC treatment, if the panel of the one or more SNPs is detected in the biological sample; or monitoring the relapse in the subject prior to two years after the discontinuation of the NUC treatment, if none of the one or more SNPs is detected in the biological sample.

39. The in vitro method of monitoring relapse of claim 38, wherein the one or more SNPs are associated with time to relapse with a p-value of 5.40E-06 or less, and the one or more SNPs are selected from the group consisting of rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542.

40-47. (canceled)

48. The in vitro method of monitoring relapse of claim 38, wherein the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, allele C in rs10235518, allele T in rs4668818, allele C in rs948006, allele A in rs2934456, allele T in rs77586835, allele C in rs75876539, allele G in rs8050261, or allele A in rs1542951.

49-50. (canceled)

51. A method for treating a chronic hepatitis B (CHB) infection in a subject in need thereof, comprising:
a. detecting in a biological sample obtained from the subject a panel of single nucleotide polymorphisms (SNPs), and the panel comprises one or more SNPs associated with time to relapse, and the one or more SNPs are selected from the group consisting of rs2154237, rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, rs231770, rs9277535, rs3130542, rs7574865, rs2296651, and rs1419881; and
b. administering to the subject a therapeutically effective amount of a nucleotide or nucleoside analogue (NUC) if the panel of the one or more SNPs is detected in the biological sample; or administering to the subject a therapeutically effective amount of a non-NUC agent if none of the SNPs is detected in the biological sample.

52. The method of claim 51, wherein the one or more SNPs are associated with time to relapse with a p-value of 5.40E-06 or less, and the one or more SNPs are selected from the group consisting of rs4315565, rs7534054, rs12105972, rs1994245, rs11896590, rs7629161, rs9828024, rs7670984, rs12645094, rs2163787, rs924446, rs180001, rs12199613, rs2394952, rs17152258, rs7459445, rs1053403, rs2767035, rs3943102, rs2154237, rs73371840, rs7205040, rs552219, rs78045374, rs117634357, rs2236895, rs7646021, rs17152247, rs10235518, rs4668818, rs948006, rs2934456, rs77586835, rs75876539, rs8050261, rs1542951, and rs3130542.

53-60. (canceled)

61. The method of claim 51, wherein the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, allele C in rs10235518, allele T in rs4668818, allele C in rs948006, allele A in rs2934456, allele T in rs77586835, allele C in rs75876539, allele G in rs8050261, or allele A in rs1542951.

62. The method of claim 61, wherein the one or more SNPs comprise allele A in rs7534054, allele A in rs4315565, allele G in rs12105972, allele T in rs1994245, allele G in rs11896590, allele T in rs7629161, allele G in rs9828024, allele C in rs7670984, allele A in rs12645094, allele G in rs2163787, allele C in rs924446, allele G in rs180001, allele C in rs12199613, allele A in rs2394952, allele C in rs17152258, allele T in rs7459445, allele G in rs1053403, allele C in rs2767035, allele C in rs3943102, allele G in rs2154237, allele C in rs73371840, allele T in rs7205040, allele A in rs552219, allele T in rs78045374, allele C in rs117634357, allele G in rs2236895, allele T in rs7646021, allele C in rs17152247, or allele C in rs10235518.

63. The method of claim 61, wherein the one or more SNPs comprise allele T in rs4668818, allele C in rs948006, allele A in rs2934456, allele T in rs77586835, allele C in rs75876539, allele G in rs8050261, or allele A in rs1542951.

64. The method of claim 51, wherein the NUC is selected from the group consisting of tenofovir, entecavir, lamivudine, adefovir, and telbivudine.

65. The method of claim 51, wherein the non-NUC agent is interferon.
